(19) United States
(12) Patent Application Publication
(10) Pub. No.: US 2025/0154221 A1
(43) Pub. Date: May 15, 2025

(54) VOLTAGE INDICATORS FOR TWO-PHOTON IMAGING

(71) Applicant: HOWARD HUGHES MEDICAL INSTITUTE, Chevy Chase, MD (US)

(72) Inventors: Ahmed Abdelfattah, Barrington, RI (US); Rosario Valenti, Buenos Aires (AR); Eric R. Schreiter, Leesburg, VA (US)

(21) Appl. No.: 18/943,722

(22) Filed: Nov. 11, 2024

Related U.S. Application Data
(60) Provisional application No. 63/597,955, filed on Nov. 10, 2023.

(51) Int. Cl. C07K 14/705; G01N 21/64

(52) U.S. Cl. CPC C07K 14/705; G01N 21/6428; G01N 21/6458; C07K 2319/60; G01N 2021/6439

(57) ABSTRACT

A voltage indicator is described, including a voltage-sensitive rhodopsin domain having mutations to replace canonical counterion charge residues with neutral residues, and a capture protein that binds a fluorescent element, wherein the capture protein is provided together with the voltage-sensitive rhodopsin domain in a fusion protein.

### RELATED APPLICATIONS

This application claims priority from U.S. Provisional Application Ser. No. 63/597,955 filed Nov. 10, 2023, the entire disclosure of which is incorporated herein by this reference.

### REFERENCE TO AN ELECTRONIC SEQUENCE LISTING

The contents of the electronic sequence listing (HHMI-2024-004-02-SequenceListing.xml]; Size: 168,056 bytes; and Date of Creation: Nov. 11, 2024) is herein incorporated by reference in its entirety.

### TECHNICAL FIELD

The presently-disclosed subject matter generally relates to voltage indicators and methods of use thereof. More specifically, the presently-disclosed subject matter relates to voltage indicators that maintain voltage sensitivity when illuminated with two-photon light and methods of measuring voltage using voltage.

### INTRODUCTION

Genetically encoded voltage indicators (GEVIs) enable optical recording of membrane potential from targeted cells in vivo, providing a less invasive method to visualize voltage dynamics in genetically defined neurons with high spatiotemporal resolution. Changes in membrane potential are fundamental to the nervous system's communication and are typically monitored with electrodes. However, GEVIs compatible with two-photon microscopy, which can be multiplexed with reporters like GCaMP, are currently lacking.

Rhodopsin protein domains are attractive GEVI scaffolds due to their extremely fast response times (<1 ms) to membrane potential changes. These proteins have been successfully combined with red-shifted fluorescent proteins or dyes via Förster resonance energy transfer (FRET) for one-photon (1P) in vivo voltage imaging. Despite their advantages, rhodopsin-based GEVIs have historically not been used with two-photon (2P) voltage imaging due to reduced voltage sensitivity under 2P illumination. Recent work has begun to explore the causes of this limitation. In contrast, the ASAP family of GEVIs, based on cpGFP and voltage-sensitive domains (VSD), has enabled two-photon voltage imaging in vivo, allowing access to cells deeper in tissue. However, optimizing analogous GEVIs based on red-shifted fluorescent proteins has proven challenging.

Microbial rhodopsin proteins, which are light-sensitive and undergo conformational changes when exposed to light, have been engineered into GEVIs. These sensors can monitor electrical activity in neurons and other excitable cells with high temporal and spatial resolution. They report membrane voltage changes through intrinsic fluorescence changes or via a reporter fluorophore using electrochromic Förster Resonance Energy Transfer (eFRET). Despite their potential, rhodopsin-based GEVIs lose voltage sensitivity under two-photon illumination, limiting their use for imaging voltage at greater depths in the brain.

Accordingly, there remains a need in the art for improved genetically-encoded voltage sensors and methods for their use.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The presently-disclosed subject matter includes voltage indicators, polypeptides, nucleotide molecules, vectors, and methods of use thereof. As disclosed herein, voltage indicators and polypeptides of the presently-disclosed subject matter include a rhodopsin domain. Nucleotide molecules and vectors as disclosed herein comprise an nucleic acid sequence encoding a rhodopsin domain.

As will be appreciated by those of ordinary skill in the art, a rhodopsin domain is a segment of a rhodopsin protein that responds to changes in membrane voltage by altering its conformation. This property can be exploited to convert a rhodopsin into a voltage sensor. In some embodiments, the rhodopsin domain is a microbial rhodopsin domain.

The presently-disclosed subject matter includes a voltage indicator, which comprises a voltage-sensitive rhodopsin domain comprising an amino acid sequence selected from the group consisting of SEQ ID NO: 1-30, including a mutation to replace a charged canonical amino acid residue with a neutral residue at one, two, or three residues within a pump or channel core motif of the rhodopsin domain. The voltage indicator also comprises a capture protein that covalently or noncovalently binds a fluorescent element that is a fluorescent protein, or a fluorescent dye. The capture protein is provided together with the voltage-sensitive rhodopsin domain in a fusion protein.

For example, X can be alanine (A), asparagine (N), cysteine (C), glutamine (Q), glycine (G), isoleucine (I), leucine (L), methionine (M), phenylalanine (F), proline (P), serine (S), threonine (T), tryptophan (W), tyrosine (Y), or valine (V).

In some embodiments, when the voltage-sensitive rhodopsin domain is from Klebsormidium nitens (KnR), the mutation can be D89X, D100X, and/or E206X where X is a neutral residue, with reference to SEQ ID NO: 1. In some embodiments, when the voltage-sensitive rhodopsin domain is from Podospora anserine (PaR), the mutation can be D136X, D147X, and/or E254X, where X is a neutral residue, with reference to SEQ ID NO: 2. In some embodiments, when the voltage-sensitive rhodopsin domain is from Haloarcula argentinensis (Cruxrhodopsin-1) (CR1), the mutation can be D83X, D94X, and/or E208X where X is a neutral residue, with reference to SEQ ID NO: 3. In some embodiments, when the voltage-sensitive rhodopsin domain is from Leptosphaeria maculans (LmR), the mutation can be D139X, D150X, and/or E258X where X is a neutral residue, with reference to SEQ ID NO: 4. In some embodiments, when the voltage-sensitive rhodopsin domain is from Neurospora crassa (NcR), the mutation can be D131X, E142X, and/or E251X where X is a neutral residue, with reference to SEQ ID NO: 5. For example, X can be A, N, C, Q, G, I, L, M, F, P, S, T, W, Y, or V.

In some embodiments, when the voltage-sensitive rhodopsin domain is from Acetabularia acetabulum (Ace1), the mutation can be D89X, D100X, and/or E206X where X is a neutral residue, with reference to SEQ ID NO: 6. In some embodiments, when the voltage-sensitive rhodopsin domain is from Chlorella vulgaris (CvR), the mutation can be D86X, D97X, and/or E203X where X is a neutral residue, with reference to SEQ ID NO: 7. In some embodiments, when the voltage-sensitive rhodopsin domain is from Taphrina deformans (TdR), the mutation can be D110X, D121X, and/or E227X where X is a neutral residue, with reference to SEQ ID NO: 8. In some embodiments, when the voltage-sensitive rhodopsin domain is from Exophiala sideris (EsR), the mutation can be D116X, D127X, and/or E233X where X is a neutral residue, with reference to SEQ ID NO: 9. In some embodiments, when the voltage-sensitive rhodopsin domain is from Moelleriella libera (MIR), the mutation can be D110X, D121X, and/or E227X where X is a neutral residue, with reference to SEQ ID NO: 10. For example, X can be A, N, C, Q, G, I, L, M, F, P, S, T, W, Y, or V.

In some embodiments, when the voltage-sensitive rhodopsin domain is from Saitoella complicate (ScR), the mutation can be D106X, E117X, and/or E222X where X is a neutral residue, with reference to SEQ ID NO: 11. In some embodiments, when the voltage-sensitive rhodopsin domain is from Rhodotorula graminis (RgR), the mutation can be D120X, E131X, and/or E237X where X is a neutral residue, with reference to SEQ ID NO: 12. In some embodiments, when the voltage-sensitive rhodopsin domain is from Rhodotorula toruloides (RtR), the mutation can be D109X, E120X, and/or E226X where X is a neutral residue, with reference to SEQ ID NO: 13. In some embodiments, when the voltage-sensitive rhodopsin domain is from Krokinobacter eikastus (KeR1), the mutation can be D88X and/or E99X where X is a neutral residue, with reference to SEQ ID NO: 14. In some embodiments, when the voltage-sensitive rhodopsin domain is from Krokinobacter eikastus (KeR2), the mutation can be D116X, D251X where X is a neutral residue, with reference to SEQ ID NO: 15.

In some embodiments, when the voltage-sensitive rhodopsin domain is from Gloeobacter violaceus (GvR), the mutation can be D121X and/or E132X where X is a neutral residue, with reference to SEQ ID NO: 16. In some embodiments, when the voltage-sensitive rhodopsin domain is from Salinibacter ruber (SrR), the mutation can be D96X and/or E107X where X is a neutral residue, with reference to SEQ ID NO: 17. In some embodiments, when the voltage-sensitive rhodopsin domain is from Gammaproteobacteria (GpR1), the mutation can be D97X and/or E108X where X is a neutral residue, with reference to SEQ ID NO: 18. In some embodiments, when the voltage-sensitive rhodopsin domain is from Gammaproteobacteria (GpR2), the mutation can be D98X and/or E109X where X is a neutral residue, with reference to SEQ ID NO: 19. In some embodiments, when the voltage-sensitive rhodopsin domain is from Proteomonas sulcate (PsuCCR), the mutation can be D121X where X is a neutral residue, with reference to SEQ ID NO: 20.

In some embodiments, when the voltage-sensitive rhodopsin domain is from Guillardia theta (GtCCR1), the mutation can be D98X where X is a neutral residue, with reference to SEQ ID NO: 21. In some embodiments, when the voltage-sensitive rhodopsin domain is from Guillardia theta (GtCCR2), the mutation can be D98X where X is a neutral residue, with reference to SEQ ID NO: 22. In some embodiments, when the voltage-sensitive rhodopsin domain is from Guillardia theta (GtCCR3), the mutation can be D110X where X is a neutral residue, with reference to SEQ ID NO: 23. In some embodiments, when the voltage-sensitive rhodopsin domain is from Guillardia theta (GtCCR4), the mutation can be D127X where X is a neutral residue, with reference to SEQ ID NO: 24. In some embodiments, when the voltage-sensitive rhodopsin domain is from Guillardia theta (GtCCR5), the mutation can be D106X where X is a neutral residue, with reference to SEQ ID NO: 25.

In some embodiments, when the voltage-sensitive rhodopsin domain is from Emiliania huxleyi (EhR), the mutation can be D88X where X is a neutral residue, with reference to SEQ ID NO: 26. In some embodiments, when the voltage-sensitive rhodopsin domain is from Actinomycetes bacterium (AbR), the mutation can be E107X where X is a neutral residue, with reference to SEQ ID NO: 27. In some embodiments, when the voltage-sensitive rhodopsin domain is from Heimdallarchaeota archaeon (HaR), the mutation can be N108X where X is a neutral residue, with reference to SEQ ID NO: 28. In some embodiments, when the voltage-sensitive rhodopsin domain is from Halobacterium sp. DLI (HdR), the mutation can be D107X where X is a neutral residue, with reference to SEQ ID NO: 29. In some embodiments, when the voltage-sensitive rhodopsin domain is from Thermoplasmatales archaeon SG8 (TsR), the mutation can be D128 where X is a neutral residue, with reference to SEQ ID NO: 30.

In some embodiments, the voltage indicator or polypeptide of the presently-disclosed subject matter comprises an amino acid sequence selected from the group consisting of SEQ ID NO: 31-74.

The voltage indicator or polypeptide of the presently-disclosed subject matter further comprises a capture protein. In some embodiments, the capture protein is a self-labeling protein or avidin.

In some embodiments, the capture protein is a self-labeling protein. In some embodiments, the fluorescent element is the fluorescent dye, and the self-labeling protein binds the fluorescent dye via a self-labeling protein ligand. In some embodiments, the SLP is a HaloTag® comprising the sequence of SEQ ID NO: 75.

In some embodiments, the voltage indicator further comprises a 1, 2, 3, 4, 5, 6, 7, 8, 9, or 10 amino acid linker between the voltage-sensitive rhodopsin domain and the capture protein.

In some embodiments, the voltage indicator further comprises a fluorescent element. In some embodiments, the fluorescent element is a fluorescent dye. In some embodiments, the fluorescent element is a fluorescent protein. In some embodiments, the fluorescent protein comprising an amino acid sequence selected from the group consisting of SEQ ID NO: 76-78.

In some embodiments, the voltage indicator or polypeptide further comprises a targeting sequence.

In some embodiments, the voltage indicator or polypeptide further comprises an endoplasmic reticulum (ER) export sequence.

In some embodiments, the voltage indicator or polypeptide further comprises an amino acid sequence selected from the group consisting of SEQ ID NO: 79-82.

In some embodiments, the voltage indicator or polypeptide comprises an amino acid sequence selected from the group consisting of SEQ ID NO: 83-135.

The presently-disclosed subject matter further includes nucleotide molecules. In some embodiments, the nucleotide molecule comprises a nucleotide encoding an amino acid sequence selected from the group consisting of SEQ ID NO: 1-30, including a mutation to replace a charged canonical amino acid residue with a neutral residue at a residue within a pump or channel core motif of the rhodopsin domain. In some embodiments, the nucleotide molecule comprises a nucleotide encoding an amino acid selected from the group consisting of SEQ ID NO: 31-74.

In some embodiments, the nucleotide molecule further comprises a nucleotide encoding an amino acid sequence for a capture protein. In some embodiments, the nucleotide molecule further comprises a nucleotide encoding an amino acid sequence of a HaloTag® comprising the sequence of SEQ ID NO: 75.

In some embodiments, the nucleotide molecule further comprises a nucleotide encoding an amino acid sequence for a fluorescent protein. In some embodiments, the nucleotide molecule comprises a nucleotide encoding an amino acid sequence for a fluorescent protein selected from the group consisting of SEQ ID NO: 76-78.

In some embodiments, the nucleotide molecule further comprises a nucleotide encoding an amino acid sequence for a targeting sequence and/or an endoplasmic reticulum (ER) export sequence. In some embodiments, the nucleotide molecule further comprises a nucleotide encoding an amino acid sequence selected from the group consisting of SEQ ID NO: 79-82.

In some embodiments, the nucleotide molecule further comprises a nucleotide encoding an amino acid sequence selected from the group consisting of SEQ ID NO: 83-135.

The presently-disclosed subject matter further includes a vector comprising a nucleotide molecule as disclosed herein.

The presently-disclosed subject matter further includes a method of measuring voltage, which comprises (i) delivering to a cell a nucleotide encoding an amino acid molecule comprising a voltage-sensitive rhodopsin domain comprising an amino acid sequence selected from the group consisting of SEQ ID NO: 1-30, including a mutation to replace a charged canonical amino acid residue with a neutral residue at one, two, or three residues within a pump or channel core motif of the rhodopsin domain; and a capture protein that covalently or noncovalently binds a fluorescent element that is a fluorescent protein, or a fluorescent dye; wherein the capture protein is provided together with the voltage-sensitive rhodopsin domain in a fusion protein; and (ii) delivering to the cell a fluorescent element that is a fluorescent protein or a fluorescent dye, wherein when the fluorescent element is a fluorescent protein, it is provided together with the capture protein and the voltage-sensitive rhodopsin domain in a fusion protein; and when the fluorescent element is a fluorescent dye, it is attached to a ligand of the capture protein; and (iii) determining changes in fluorescence of the fluorescent element, as an measurement of voltage changes in the cell, wherein an increase in membrane potential lead to an increase in fluorescence.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1A-1F. Screening diverse opsin proteins for voltage sensitivity under two photon (2P). FIG. 1A. Schematic of experimental design. Sequences of diverse rhodopsins are aligned, homologous amino acid positions are mutated to engineer possible voltage indicator with no native pump/channel function. Rhodopsins are then fused to HaloTag, expressed in neuron culture and labelled with JF₅₂₅-HaloTag ligand to check for membrane trafficking, 1P voltage sensitivity and 2P voltage sensitivity. FIG. 1B. Left: cartoon of Ace2 rhodopsin domain (PDB ID: 3AM6). Right: zoom in on retinal binding pocket in Ace2 with surrounding amino acids. Residues are shaded based on their chemical properties. FIG. 1C. Comparison of key residues in the binding pocket of Ace2 rhodopsin, different classes of rhodopsins tested for voltage sensitivity, Klebsormidium nitens rhodopsin (KnR), and Podospora anserine rhodopsin (PaR). Residues are shaded according to their chemical properties as in 1B. FIG. 1D. Fluorescence response (ΔF/F₀) of different rhodopsin-HaloTag fusions with 1P illumination (514 nm) to elicited action potentials. Cultured neurons are transfected and labeled with JF₅₂₅-HaloTag ligand and stimulated using field potentials to elicit a train of 10 action potentials at 66 Hz. Top: Fluorescence response of opsin domains with amino acid mutations to develop a negative going FRET signal. Middle: Fluorescence response of opsin domains with amino acid mutations to develop a negative going FRET signal. Bottom, phylogeny tree of the rhodopsins tested, based on opsin domain sequence alignments. The scale indicates the number of substitutions per site. FIG. 1E. Fluorescence response (ΔF/F₀) of different rhodopsin-HaloTag fusions with 2P illumination (1030 nm) to elicited action potentials. Cultured neurons are transfected and labeled with JF₅₂₅-HaloTag ligand and stimulated using field potentials to elicit a train of 10 action potentials at 66 Hz. (−) and (+) signs indicate negative-going or positive-going version of the rhodopsin-HaloTag fusion respectively. FIG. 1F. Fluorescence image of KnR-HT-ST (2Photron-ST) (top) and PaR-HT-ST (bottom) labeled with JF₅₂₅ HaloTag ligand in a hippocampal neuron in culture. Scale bar: 20 μm. FIG. 1G. Fluorescence response of 2Photron-ST₅₂₅ and PaR-HT-ST₅₂₅ (bottom) in cultured hippocampal neurons to a train of field stimuli eliciting 10 APs at 66 Hz with 1P (514 nm) or 2P (1030 nm) illumination. Field stimulus timing is shown with black bar.

FIG. 2. Expression of negative-going voltage sensors under 1P illumination. Fluorescence images of hippocampal neurons in culture expressing rhodopsin-HaloTag fusions with point mutations to generate negative-going voltage sensors. Scale bar: 50 μm.

FIG. 3. Expression of positive-going voltage sensors under 1P illumination. Fluorescence images of hippocampal neurons in culture expressing rhodopsin-HaloTag fusions with point mutations to generate negative-going voltage sensors. Scale bar: 50 μm.

FIG. 4. Example fluorescence response of rhodopsin-HaloTag fusions that exhibited fluorescence change to field stimulation. Top Left: Schematic of field stimulation protocol. Left: Fluorescence images of hippocampal neurons in culture expressing rhodopsin-HaloTag fusions. Scale bar: 50 μm. Right: Fluorescence response to 66 Hz field stimulation.

FIG. 5A-5C. Trafficking of Podospora and Neurospora rhodopsin domains fused to HaloTag in neuron culture. FIG. 5A. Podospora anserine rhodopsin when fused with HaloTag and labeled with JF₅₂₅ displayed poor membrane trafficking in neuron culture. FIG. 5. Neurospora crassa rhodopsin when fused with HaloTag and labeled with JF₅₂₅ displayed good membrane trafficking in neuron culture. FIG. 5C. Replacing the N-terminal 44 residues of Podospora rhodopsin with the N-terminal 39 residues from Neurospora rhodopsin improved better membrane trafficking. Scale bar: 40 μm.

FIG. 6. Amino acid sequence of KnR-HT (SEQ ID NO: 84) (Top) and PaR-HT (SEQ ID NO: 90) (bottom), with sequence features annotated.

FIG. 7A-7E. In vitro characterization of new rhodopsin GEVIs. FIG. 7A. Representative fluorescence traces of KnR-HT₅₅₂ (top) in response to a series of 0.5 s voltage steps (bottom: from −145 mV to +55 mV in 25 mV increments) under 1P and 2P excitation. FIG. 7B. Fluorescence changes as a function of membrane voltage with KnR-HT₅₅₂ and PaR-HT₅₅₂ under 1P and 2P excitation. Errors bars are S.D., n=12 (KnR-HT₅₅₂) and 10 (PaR-HT₅₅₂) cells. FIG. 7C. Fluorescence response of KnR-HT₅₅₂ to a 150 mV voltage step under 1P (orange, thin line) and 2P (purple, thin line) excitation. Fluorescence traces were averaged from 5 recordings and fit using a double exponential function (thick lines). FIG. 7D. Single-trial recordings of action potentials and subthreshold membrane potentials induced by current injections in primary neurons expressing KnR-HT₅₅₂ (top) or PaR-HT₅₅₂ (bottom) with 1P and 2P imaging at 500 Hz (greyscale traces) or electrophysiology (black traces). Inset: fluorescence images of primary neurons expressing KnR-HT₅₅₂ or PaR-HT₅₅₂. Scale bar: 20 μm. FIG. 7E. Comparison of spike amplitude of KnR-HT₅₅₂ (top) or PaR-HT₅₅₂ (bottom) under 1P and 2P imaging at 500 Hz; n=4 cells for KnR-HT₅₅₂ and n=3 cells for PaR-HT₅₅₂; ns indicates not significant (p>0.05). This measures only the spike amplitude, the underlying subthreshold signal is not included.

FIG. 8. Characterization of the kinetic properties of KnR-HT (2Photron) under 1P illumination. Left: Representative fluorescence response of 2Photron labeled with JF552-HTL in a cultured neuron to a 150 mV potential step delivered in voltage clamp. Insets: Zoom in on change of 2Photron fluorescence to depolarization and hyperpolarization. Solid line is double exponential fit according to ΔF/F(t)=A1*e^{-t/τ fast}+A2*e^{-t/τ slow}. Image acquisition rate was 3.2 kHz. Right: Quantification of 2Photron kinetics in primary neurons in culture. Errors are S.D. n=6 cells.

FIG. 9A-9F. Rhodopsin derived GEVIs are generally compatible with 2p excitation. Single-trial recordings of action potentials and subthreshold voltage signals from current injections in cultured primary rat hippocampal neurons expressing Voltron (FIG. 9A), Positron2 (FIG. 9B), Voltron2 (FIG. 9C) or QuasAr2-HT (FIG. 9D) and labeled with JF552-HTL, using 500-Hz imaging (top, fluorescence) or electrophysiology (bottom, membrane potential) under sequential 1p and stationary 2p illumination. Fluorescence changes as a function of membrane voltage with Voltron2 (FIG. 9E) and QuasAr2 (FIG. 9F) under 1p and 2p light excitation. Scale bar: 20 μm. Errors are S.D. n=7 (FIG. 9E) or 5 (FIG. 9F) cells.

FIG. 10A-10B. KnR-HT and Voltron2 are compatible with point scanning 2p excitation. Single-trial recordings of action potentials and subthreshold voltage signals from current injections in cultured primary rat hippocampal neurons expressing 2Photron (FIG. 10A) and Voltron2 (FIG. 10B) and labeled with JF552-HTL, using 500 Hz imaging (top and middle: fluorescence) or electrophysiology (bottom: membrane potential) under sequential 1P (left) and circular point scanning 2P illumination (right). Scale bar: 20 μm.

FIG. 11A-11G. 2Photron enables monitoring of sub and suprathreshold voltage in behaving mice. FIG. 11A. Left, experimental design of dual-excitation path configuration to simultaneously record 2Photron-ST₅₅₂ and a green emission GEVI or a GECI in behaving mice. Right, fluorescence images of a cortical neuron co-expressing 2Photron-ST₅₅₂ and JEDI-2P-Kv Scale bar: 10 μm. FIG. 11B. Representative ULoVE 3.75 KHz fluorescence trace from a cortical neuron expressing 2Photron-ST₅₅₂ in an awake, behaving mouse. Below, zoom of the trace at the beginning (i) and at the end (ii) of the recording showing the acquisition of spikes and subthreshold potentials. FIG. 11C. Representative dual recording of 2Photron-ST₅₅₂ (top) and JEDI-2P-Kv (bottom). Black traces are low-pass filtered. FIG. 11D. Distribution of the 2Photron-ST₅₅₂ signal as a function of the JEDI-2P-Kv signal. The data-point density is grey shaded, and a linear regression is shown in red. Slope of the regression is 0.32±0.096 (n=13 cells, n=4 mice). FIG. 11E. Normalized average spike waveforms from two cells co-expressing 2Photron-ST₅₅₂ (red) and JEDI-2P-Kv (green) having an after-depolarization (top) and an after-hyperpolarization (shaded area, bottom). Quantifications are given as mean±std for the exponential time constant of the depolarization (middle) and repolarization (right). Exponential time constant provided as mean±std [min-max], for depolarization: 2Photron-ST₅₅₂: 0.38±0.27 ms [0.27-0.55 ms], JEDI-2P-Kv: 0.77±0.04 [0.71-0.83 ms]. Wilcoxon rank sum test: p=4.11e-5; for repolarization: 2Photron-ST₅₅₂: 0.77±0.47 ms [0.37-1.86 ms], JEDI-2P-Kv: 2.60±0.39 ms [2.00-3.20 ms]. Wilcoxon rank sum test: p=1.55e-4. The 2Photron-ST₅₅₂ post-spike repolarization level reaches 19.60±4.23% of the JEDI post-spike repolarization level at 7.80±6.92 ms. *** indicates p<0.001, data from n=9 cells, n=4 mice. FIG. 11F. Comparison of 2Photron-ST₅₅₂ (red) with ASAP3 (grey) and JEDI-2P-Kv (green); quantifications of spike amplitude, spike FWHM, and photon flux are represented with bars±std. *** indicates p<0.001. ASAP3 data are from Villette et al., 2019, n=23 cells. JEDI-2P-Kv data are from Liu et al., 2022, n=34 cells. 2Photron-ST₅₅₂ (n=16 cells from 5 mice. FIG. 11G. Average single spikes from the 25 2Photron-ST₅₅₂ recorded cells sorted top to bottom as a function of spike SNR. Example cells from FIGS. 11B and 11C are labeled with arrowheads.

FIG. 12A-12D. 2Photron-ST₅₅₂ outperforms Voltron2-ST₅₅₂ recording from fast-spiking cerebellar granular layer glycinergic interneurons in vivo. FIG. 12A. Representative single 2P-image planes of GEVI expression in cerebellar Glyt2 positive granular layer neurons in vivo. Top, JEDI2P. Middle, Voltron2-ST₅₅₂. Bottom, 2Photron-ST₅₅₂. FIG. 12B. ULoVE recordings from cells in A at >3.5 KHz in awake-behaving mice. FIG. 12C. Average spike waveforms from cells in B (mean±sem). Note the depolarization ramp preceding and the afterhyperpolarization (AHP) following the spike. FIG. 12D. Comparisons with the state-of-the-art GEVIs. Quantifications of spike amplitude, afterhyperpolarization potential (AHP), the dynamic range, spike FWHM, the recorded photon flux, the spike Signal to Noise ratio (SNR), and the spike detectability index (D′). Green, JEDI-2P-Kv, n=10, Red, Voltron2-ST₅₅₂, n=11. Black, 2Photron-ST₅₅₂ n=19. ±std, *** indicates p<0.001, ** indicates p<0.01 (ranksum test).

FIG. 13A-13D. Method for multispectral two-photon optical recordings without spectral crosstalk. FIG. 13A. Timeline of experimentation: virus injection preceded 2P-experiments by 30-40 days while JF552 fluorophore is injected 1 or 2 days before performing optical recordings. FIG. 13B. Left, experimental scheme and filter setting for cell recording. Right, single 2P-image planes of expression in vivo. FIG. 13C. Illustration of ULoVE excitation pattern positioning in space (top) and time (middle) used to obtain optical voltage records (bottom): Top row indicates how the four ULoVE patterns (70 us per pattern) are spatially placed for a single acquisition, two ULoVE patterns per cell are first applied through the 900-1200 nm AOD path then two other ULoVE patterns are applied through the 800-1000 nm path. To avoid cross-contamination of the signal pathways, patterns are alternately spatially offset and laser excitation is off. FIG. 13D. Normalized photon flux (n=14 cells) showing the absence of bleed-through from the 920 nm excitation into the 572-642 nm PMT, controlling for cross-contamination signals.

FIG. 14A-14F. GEVI co-expression and ground truth calibration to determine ideal photon flux. FIG. 14A. Full frame single-plane 2P images in vivo of 2Photron-ST₅₅₂ (left) and JEDI-2P-Kv (right), scale bar is 50 μm and depth is 214 μm below the meninges. FIG. 14B. Zoomed images for brightness levels of the different GEVIs, note differences of grey scales. i,ii, and iii are examples displayed in the panel c, respectively high 2Photron-ST₅₅₂ with very low JEDI-2P, low 2Photron-ST₅₅₂ and low JEDI-2P, and average brightness levels for 2Photron-ST₅₅₂ and high JEDI-2P. FIG. 14C. Estimated 2Photron-ST₅₅₂ and JEDI-2P photon fluxes (n=403 neurons, 4 mice, depth: 165.62±70.4 μm). Greys represent the log 10 of flux ratio. Dashed line log 10 flux ratio of 0. Note neurons expressing workable JEDI-2-Kv photon flux display only a weak 2Photron-ST₅₅₂ photon flux. Right, zoom where the magenta line represents the principal component of high ratio data points (above 5 folds). FIG. 14D. Mean photon flux (JEDI-2-Kv black, 2Photron-ST₅₅₂ red dots) as a function of JEDI-2-Kv flux expressed in percentile. Interestingly, we found: i) a correlation between JEDI2P-Kv and 2Photron-ST₅₅₂ photon fluxes. This correlation holds for the 96.3 first percent of the distribution (388/403 neurons, Pearson correlation coefficient: r=0.92422, p-value 1.2623e-16, slope (magenta): 0.304 MHz/10% of ranked cell), ii) within the 3.7 last percent of the distribution when the JEDI-2P-Kv reached its highest flux, we found a sudden drop of the 2Photron-ST₅₅₂ photon flux (15/403 neurons, blue shaded area). When compared to the value predicted by the regression (thin magenta line), this drop corresponds to a flux decrease of 38.46% (predicted photon flux: 3.7 MHz, measured photon flux: 2.28 MHz). Note: for reasons still unclear, however, the highest expression of either indicator was not observed in cells of co-expression. Thus, cells of the highest JEDI2P-Kv expression were chosen to establish the ground truth spiking activity, even though this meant they are amongst the lower level of 2Photron co-expression and consequently limited the epochs of comparison to the first minute of the 10-minute recording. FIG. 14E. Measurements of Error rate. Sensitivity, Specificity, and Precision from spikes during the first minute of the JEDI-2P-Kv and 2Photron-ST₅₅₂ traces (n=9 cells, 4 mice). The error rate corresponds to the harmonic average of the sensitivity and the precision. Note that error rate values (sorted by gray shading) correlate with the overall metrics. FIG. 14F. Limit of the photon flux that enables a good theoretical detection. Plot of error rate as a function of average photon flux during the first minute of recordings. Note: regardless of cell firing rate, a lower error rate correlates with a higher photon flux. Linear regression projected to y=0 gives a limit of 3.24 MHz. Note that this limit of theoretically perfect detection is well below the distribution of the ULoVE-based 2Photron-ST₅₅₂ photon fluxes extracted from the 2Photron-ST₅₅₂/GCaMP experiments (n=16 neurons).

FIG. 15A-15L. Multiplexed 2Photron-ST₅₅₂ and GCaMP recordings reveal contributions from spiking and subthreshold voltage dynamics to calcium transient amplitudes. FIG. 15A. Single 2P-image planes showing 2Photron-ST₅₅₂ (top) and GCaMP6f (bottom) expression in mouse cortex, scale bar 5 μm. FIG. 15B. Representative multiplexed 2Photron-ST₅₅₂ (top) and GCaMP6f (bottom) recording. Right, zoom of recording within the dashed box encompassing a burst. FIG. 15C, 15D. same as (FIG. 15A) and (FIG. 15B) but using GCaMP8f. FIG. 15E. Average bursts where each 2Photron-ST₅₅₂ spike (red trace) is a mean of spikes within the burst. Black trace, drifting average MLspike baseline spike trigger signal. Green trace, average calcium transient. Filled area is mean±std. FIG. 15F. Top, 2Photron-ST₅₅₂ signal for depolarizations without spikes (blue, n=119), depolarizations with spikes (black, n=164), and depolarizations with spikes selected to match subthreshold depolarizations (red n=52). Bottom, corresponding GCaMP8f signals, without spikes: 0.03±0.46% ΔF/F, with spikes: 23.2±0.18% ΔF/F. FIG. 15G. Distributions of GCaMP fluorescence amplitudes as a function of spike burst size for individual cells, shaded by the scaling factor obtained from quadratic fits. FIG. 15H. Cumulative distributions and sigmoid fit of GCaMP8f calcium transient amplitudes as a function of burst sizes. Left, before normalization. Right, after normalization. FIG. 15I. Probability distribution of burst occurrence as a function of the normalized amplitude. FIG. 15J. Cumulative spike distribution as a function of the normalized amplitude. Black, true positive. Grey: false positive, ranging in shades of grey from ±1 to 4 erroneous spikes. FIG. 15K. Three example bursts of five spikes from the same neuron (2Photron-ST₅₅₂, top), highlighting variation in GCaMP fluorescence response (bottom). Indicated values for voltage traces are mean intraburst firing frequency, average 2Photron-ST₅₅₂ signal at burst onset, and burst duration. GCaMP responses sorted according to peak amplitude, noted as a fraction of the mean response for this cell and spike burst size. FIG. 15L. Normalized calcium transient peak amplitudes as a function of the three voltage-dependent metrics measured in (FIG. 15K). Lines represent significant correlations with the relative amplitude of the GCaMP signal.

FIG. 16A-16J. Normalizations and distributions applied to data of Zhang et al., 2023. FIG. 16A. Calcium transient peak amplitude distribution versus burst size. White, GCaMP6f, green, GCaMP8f. Red, quadratic fit for GCaMP8f data. FIG. 16B. Mean burst size coefficient of variation. White, individual cells, Dark grey, cells pooled together, Light grey cells normalized and pooled). Rank sum test: **p value<0.01, *p value<0.05. error+/−std. FIG. 16C and FIG. 16D, As for FIG. 15H normalizing on the largest event of the cell without considering the burst size distribution. FIG. 16E, As for FIG. 15J using the distribution in FIG. 16D. FIG. 16F, Zhang et al., 2023 calcium transient amplitudes as a function of burst size for 20 individual GCaMP8f cells. Shading is according to a scaling factor obtained from quadratic fit, and ranged from 14 to 136 (mean+/−std: 46.4+/−26.5). FIG. 16G. As for FIG. 15H, using data taken from Zhang et al., 2023. Left, before normalization (CV: 0.569). Right, after normalization (CV: 0.279). FIG. 16H. As for FIG. 15, using data taken from Zhang et al., 2023. Despite fewer burst sizes, comparable results are obtained for true positive and average spike error per event: 68.9% and 0.291 (before normalization), 77.8% and 0.214 (after normalization), and 73% and 0.292 (for the normalization performed without knowing the burst size). FIG. 16I. Distribution of the associated 2Photron-ST₅₅₂ signals preceding the last spike of the burst. Red, sigmoid fit indicates that the spikes saturated after 5.01 spikes. FIG. 16J. Normalized calcium transient peak amplitude as a function of the interburst interval in the 2Photron-GCaMP8f dataset. Datapoints are shaded according to the neuron number from FIG. 15G.

### BRIEF DESCRIPTION OF THE SEQUENCE LISTING

SEQ ID NO: 1 is the amino acid sequence of a voltage-sensitive rhodopsin domain from Klebsormidium nitens (KnR).

SEQ ID NO: 2 is the amino acid sequence of a voltage-sensitive rhodopsin domain from Podospora anserine (PaR).

SEQ ID NO: 3 is the amino acid sequence of a voltage-sensitive rhodopsin domain from Haloarcula argentinensis (Cruxrhodopsin-1) (CR1).

SEQ ID NO: 4 is the amino acid sequence of a voltage-sensitive rhodopsin domain from Leptosphaeria maculans (LmR).

SEQ ID NO: 5 is the amino acid sequence of a voltage-sensitive rhodopsin domain from Neurospora crassa (wild type) (NcR).

SEQ ID NO: 6 is the amino acid sequence of a voltage-sensitive rhodopsin domain from Acetabularia acetabulum (Ace1).

SEQ ID NO: 7 is the amino acid sequence of a voltage-sensitive rhodopsin domain from Chlorella vulgaris (CvR).

SEQ ID NO: 8 is the amino acid sequence of a voltage-sensitive rhodopsin domain from Taphrina deformans (TdR).

SEQ ID NO: 9 is the amino acid sequence of a voltage-sensitive rhodopsin domain from Exophiala sideris (EsR).

SEQ ID NO: 10 is the amino acid sequence of a voltage-sensitive rhodopsin domain from Moelleriella libera (MIR).

SEQ ID NO: 11 is the amino acid sequence of a voltage-sensitive rhodopsin domain from Saitoella complicate (ScR).

SEQ ID NO: 12 is the amino acid sequence of a voltage-sensitive rhodopsin domain from Rhodotorula graminis (RgR).

SEQ ID NO: 13 is the amino acid sequence of a voltage-sensitive rhodopsin domain from Rhodotorula toruloides (RtR).

SEQ ID NO: 14 is the amino acid sequence of a voltage-sensitive rhodopsin domain from Krokinobacter eikastus (KeR1).

SEQ ID NO: 15 is the amino acid sequence of a voltage-sensitive rhodopsin domain from Krokinobacter eikastus (KeR2).

SEQ ID NO: 16 is the amino acid sequence of a voltage-sensitive rhodopsin domain from Gloeobacter violaceus (GvR).

SEQ ID NO: 17 is the amino acid sequence of a voltage-sensitive rhodopsin domain from Salinibacter ruber (SrR).

SEQ ID NO: 18 is the amino acid sequence of a voltage-sensitive rhodopsin domain from Gammaproteobacteria (GpR1).

SEQ ID NO: 19 is the amino acid sequence of a voltage-sensitive rhodopsin domain from Gammaproteobacteria (GpR2).

SEQ ID NO: 20 is the amino acid sequence of a voltage-sensitive rhodopsin domain from Proteomonas sulcate (PsuCCR).

SEQ ID NO: 21 is the amino acid sequence of a voltage-sensitive rhodopsin domain from Guillardia theta (GtCCR1).

SEQ ID NO: 22 is the amino acid sequence of a voltage-sensitive rhodopsin domain from Guillardia theta (GtCCR2).

SEQ ID NO: 23 is the amino acid sequence of a voltage-sensitive rhodopsin domain from Guillardia theta (GtCCR3).

SEQ ID NO: 24 is the amino acid sequence of a voltage-sensitive rhodopsin domain from Guillardia theta (GtCCR4).

SEQ ID NO: 25 is the amino acid sequence of a voltage-sensitive rhodopsin domain from Guillardia theta (GtCCR5).

SEQ ID NO: 26 is the amino acid sequence of a voltage-sensitive rhodopsin domain from Actinomycetes bacterium (AbR).

SEQ ID NO: 27 is the amino acid sequence of a voltage-sensitive rhodopsin domain from Emiliania huxleyi (EhR).

SEQ ID NO: 28 is the amino acid sequence of a voltage-sensitive rhodopsin domain from Heimdallarchaeota archaeon (HaR).

SEQ ID NO: 29 is the amino acid sequence of a voltage-sensitive rhodopsin domain from Halobacterium sp. DLI (HdR).

SEQ ID NO: 30 is the amino acid sequence of a voltage-sensitive rhodopsin domain from Thermoplasmatales archaeon SG8 (TsR).

SEQ ID NO: 31 is the amino acid sequence of a voltage-sensitive rhodopsin domain from Klebsormidium nitens (KnR (−)) having a D89N mutation.

SEQ ID NO: 32 is the amino acid sequence of a voltage-sensitive rhodopsin domain from Podospora anserine (PaR (−)) having a D136N mutation.

SEQ ID NO: 33 is the amino acid sequence of a voltage-sensitive rhodopsin domain from Podospora anserine (PaR (−)) having a D136N mutation and where residues 1-44 have been replaced with an N-terminal sequence having the amino acid sequence of SEQ ID NO: 79.

SEQ ID NO: 34 is the amino acid sequence of a voltage-sensitive rhodopsin domain from Haloarcula argentinensis (Cruxrhodopsin-1) (CR1 (−)) having a D83N mutation.

SEQ ID NO: 35 is the amino acid sequence of a voltage-sensitive rhodopsin domain from Leptosphaeria maculans (LmR (−)) having a D139N mutation.

SEQ ID NO: 36 is the amino acid sequence of a voltage-sensitive rhodopsin domain from Neurospora crassa (NcR (−)) having a D131N mutation

SEQ ID NO: 37 is the amino acid sequence of a voltage-sensitive rhodopsin domain from Acetabularia acetabulum (Ace1 (−)) having a D89N mutation.

SEQ ID NO: 38 is the amino acid sequence of a voltage-sensitive rhodopsin domain from Chlorella vulgaris (CvR (−)) having a D86N mutation.

SEQ ID NO: 39 is the amino acid sequence of a voltage-sensitive rhodopsin domain from Taphrina deformans (TdR (−)) having a D110N mutation.

SEQ ID NO: 40 is the amino acid sequence of a voltage-sensitive rhodopsin domain from Exophiala sideris (EsR (−)) having a D127N mutation.

SEQ ID NO: 41 is the amino acid sequence of a voltage-sensitive rhodopsin domain from Moelleriella libera (MIR (−)) having a D121N mutation.

SEQ ID NO: 42 is the amino acid sequence of a voltage-sensitive rhodopsin domain from Saitoella complicate (ScR (−)) having a D106N mutation.

SEQ ID NO: 43 is the amino acid sequence of a voltage-sensitive rhodopsin domain from Rhodotorula graminis (RgR (−)) having a D120N mutation.

SEQ ID NO: 44 is the amino acid sequence of a voltage-sensitive rhodopsin domain from Rhodotorula toruloides (RtR (−)) having a D109N mutation.

SEQ ID NO: 45 is the amino acid sequence of a voltage-sensitive rhodopsin domain from Gammaproteobacteria (GpR1 (−)) having a D97N mutation.

SEQ ID NO: 46 is the amino acid sequence of a voltage-sensitive rhodopsin domain from Gammaproteobacteria (GpR2 (−)) having a D98N mutation.

SEQ ID NO: 47 is the amino acid sequence of a voltage-sensitive rhodopsin domain from Krokinobacter eikastus (KeR1 (−)) having a D88N mutation.

SEQ ID NO: 48 is the amino acid sequence of a voltage-sensitive rhodopsin domain from Krokinobacter eikastus (KeR2 (−)) having a D116N mutation.

SEQ ID NO: 49 is the amino acid sequence of a voltage-sensitive rhodopsin domain from Gloeobacter violaceus (GvR (−)) having a D121N mutation.

SEQ ID NO: 50 is the amino acid sequence of a voltage-sensitive rhodopsin domain from Salinibacter ruber (SrR (−)) having a D96N mutation.

SEQ ID NO: 51 is the amino acid sequence of a voltage-sensitive rhodopsin domain from Actinomycetes bacterium (AbR) having an E107Q mutation.

SEQ ID NO: 52 is the amino acid sequence of a voltage-sensitive rhodopsin domain from Haloarcula argentinensis (Cruxrhodopsin-1) (CR1 (+)) having D94N and E208V mutations.

SEQ ID NO: 53 is the amino acid sequence of a voltage-sensitive rhodopsin domain from Leptosphaeria maculans (LmR (+)) having D150N and E258V mutations.

SEQ ID NO: 54 is the amino acid sequence of a voltage-sensitive rhodopsin domain from Podospora anserine (PaR (+)) D147N and E254V mutations.

SEQ ID NO: 55 is the amino acid sequence of a voltage-sensitive rhodopsin domain from Neurospora crassa (NcR (+)) having E142Q and E251V mutations.

SEQ ID NO: 56 is the amino acid sequence of a voltage-sensitive rhodopsin domain from Acetabularia acetabulum (Ace1 (+)) having D100N and E206V mutations.

SEQ ID NO: 57 is the amino acid sequence of a voltage-sensitive rhodopsin domain from Chlorella vulgaris (CvR (+)) having D97N and E203V mutations.

SEQ ID NO: 58 is the amino acid sequence of a voltage-sensitive rhodopsin domain from Klebsormidium nitens (KnR (+)) having D100N and E206V mutations.

SEQ ID NO: 59 is the amino acid sequence of a voltage-sensitive rhodopsin domain from Taphrina deformans (TdR (+)) having D121N and E227V mutations.

SEQ ID NO: 60 is the amino acid sequence of a voltage-sensitive rhodopsin domain from Exophiala sideris (EsR (+)) having D127N and E233V mutations.

SEQ ID NO: 61 is the amino acid sequence of a voltage-sensitive rhodopsin domain from Moelleriella libera (MIR (+)) having D121N and E227V mutations.

SEQ ID NO: 62 is the amino acid sequence of a voltage-sensitive rhodopsin domain from Saitoella complicate (ScR (+)) having E117Q and E222V mutations.

SEQ ID NO: 63 is the amino acid sequence of a voltage-sensitive rhodopsin domain from Rhodotorula graminis (RgR (+)) having E131Q and E237V mutations.

SEQ ID NO: 64 is the amino acid sequence of a voltage-sensitive rhodopsin domain from Rhodotorula toruloides (RtR (+)) having E120Q and E226V mutations.

SEQ ID NO: 65 is the amino acid sequence of a voltage-sensitive rhodopsin domain from Krokinobacter eikastus (KeR1 (+)) having an E99Q mutation.

SEQ ID NO: 66 is the amino acid sequence of a voltage-sensitive rhodopsin domain from Krokinobacter eikastus (KeR2 (+)) having a D251N mutation.

SEQ ID NO: 67 is the amino acid sequence of a voltage-sensitive rhodopsin domain from Gloeobacter violaceus (GvR (+)) having a E132Q mutation.

SEQ ID NO: 68 is the amino acid sequence of a voltage-sensitive rhodopsin domain from Salinibacter ruber (SrR (+)) having an E107Q mutation.

SEQ ID NO: 69 is the amino acid sequence of a voltage-sensitive rhodopsin domain from Proteomonas sulcate (PsuCCR (+)) having a D121N mutation.

SEQ ID NO: 70 is the amino acid sequence of a voltage-sensitive rhodopsin domain from Guillardia theta (GtCCR1 (+)) having a D98N mutation.

SEQ ID NO: 71 is the amino acid sequence of a voltage-sensitive rhodopsin domain from Guillardia theta (GtCCR2 (+)) having a D98N mutation.

SEQ ID NO: 72 is the amino acid sequence of a voltage-sensitive rhodopsin domain from Guillardia theta (GtCCR3 (+)) having a D110N mutation.

SEQ ID NO: 73 is the amino acid sequence of a voltage-sensitive rhodopsin domain from Guillardia theta (GtCCR4 (+)) having a D127N mutation.

SEQ ID NO: 74 is the amino acid sequence of a voltage-sensitive rhodopsin domain from Guillardia theta (GtCCR5) having a D106N mutation.

SEQ ID NO: 75 is the amino acid sequence of a HaloTag® protein.

SEQ ID NO: 76 is the amino acid sequence of a cyan-excitable orange fluorescent protein.

SEQ ID NO: 77 is the amino acid sequence of a monomeric orange fluorescent protein.

SEQ ID NO: 78 is the amino acid sequence of an mVenus fluorescent protein.

SEQ ID NO: 79 is the amino acid sequence of an N-terminal sequence to improve trafficking to membrane.

SEQ ID NO: 80 is the amino acid sequence of a membrane targeting sequence.

SEQ ID NO: 81 is the amino acid sequence of an endoplasmic reticulum (ER) export sequence.

SEQ ID NO: 82 is the amino acid sequence of a soma targeting sequence.

SEQ ID NO: 83 is an amino acid sequence of an exemplary voltage indicator provided in accordance with the presently disclosed subject matter, including a voltage-sensitive rhodopsin domain from Klebsormidium nitens having a D89N mutation, a HaloTag® protein, a membrane targeting sequence, and an ER export sequence.

SEQ ID NO: 84 is an amino acid sequence of an exemplary voltage indicator provided in accordance with the presently disclosed subject matter, including a voltage-sensitive rhodopsin domain from Klebsormidium nitens having a D89N mutation, a HaloTag® protein, a membrane targeting sequence, an ER export sequence, and a soma targeting sequence.

SEQ ID NO: 85 is the amino acid sequence of an exemplary voltage indicator provided in accordance with the presently disclosed subject matter, including a voltage-sensitive rhodopsin domain from Klebsormidium nitens having a D89N mutation, cyan-excitable orange fluorescent protein, a membrane targeting sequence, and an ER export sequence.

SEQ ID NO: 86 is the amino acid sequence of an exemplary voltage indicator provided in accordance with the presently disclosed subject matter, including a voltage-sensitive rhodopsin domain from Klebsormidium nitens having a D89N mutation, monomeric orange fluorescent protein, a membrane targeting sequence, and an ER export sequence.

SEQ ID NO: 87 is the amino acid sequence of an exemplary voltage indicator provided in accordance with the presently disclosed subject matter, including a voltage-sensitive rhodopsin domain from Klebsormidium nitens having a D89N mutation, mVenus fluorescent protein, a membrane targeting sequence, and an ER export sequence.

SEQ ID NO: 88 is the amino acid sequence of is an amino acid sequence of an exemplary voltage indicator provided in accordance with the presently disclosed subject matter, including a voltage-sensitive rhodopsin domain from Podospora anserine having a D136N mutation, a HaloTag® protein, a membrane targeting sequence, and an ER export sequence.

SEQ ID NO: 89 is the amino acid sequence of is an amino acid sequence of an exemplary voltage indicator provided in accordance with the presently disclosed subject matter, including a voltage-sensitive rhodopsin domain from Podospora anserine having a D136N mutation, a HaloTag® protein, a membrane targeting sequence, an ER export sequence, and a soma targeting sequence.

SEQ ID NO: 90 is the amino acid sequence of an exemplary voltage indicator provided in accordance with the presently disclosed subject matter, including a voltage-sensitive rhodopsin domain from Podospora anserine having a D136N mutation and where residues 1-44 have been replaced with an N-terminal sequence having the amino acid sequence of SEQ ID NO: 79, a HaloTag® protein, a membrane targeting sequence, an ER export sequence, and a soma targeting sequence.

SEQ ID NO: 91 is the amino acid sequence of an exemplary voltage indicator provided in accordance with the presently disclosed subject matter, including a voltage-sensitive rhodopsin domain from Haloarcula argentinensis (Cruxrhodopsin-1) (CR1 (−)) having a D83N mutation, a HaloTag® protein, a membrane targeting sequence, and an ER export sequence.

SEQ ID NO: 92 is the amino acid sequence of an exemplary voltage indicator provided in accordance with the presently disclosed subject matter, including a voltage-sensitive rhodopsin domain from Leptosphaeria maculans (LmR (−)) having a D139N mutation, a HaloTag® protein, a membrane targeting sequence, and an ER export sequence.

SEQ ID NO: 93 is the amino acid sequence of an exemplary voltage indicator provided in accordance with the presently disclosed subject matter, including a voltage-sensitive rhodopsin domain from Neurospora crassa (NcR (−)) having a D131N mutation, a HaloTag® protein, a membrane targeting sequence, and an ER export sequence.

SEQ ID NO: 94 is the amino acid sequence of an exemplary voltage indicator provided in accordance with the presently disclosed subject matter, including a voltage-sensitive rhodopsin domain from Acetabularia acetabulum (Ace1 (−)) having a D89N mutation, a HaloTag® protein, a membrane targeting sequence, and an ER export sequence.

SEQ ID NO: 95 is the amino acid sequence of an exemplary voltage indicator provided in accordance with the presently disclosed subject matter, including a voltage-sensitive rhodopsin domain from Chlorella vulgaris (CvR (−)) having a D86N mutation, a HaloTag® protein, a membrane targeting sequence, and an ER export sequence.

SEQ ID NO: 96 is the amino acid sequence of an exemplary voltage indicator provided in accordance with the presently disclosed subject matter, including a voltage-sensitive rhodopsin domain from Taphrina deformans (TdR (−)) having a D110N mutation, a HaloTag® protein, a membrane targeting sequence, and an ER export sequence.

SEQ ID NO: 97 is the amino acid sequence of an exemplary voltage indicator provided in accordance with the presently disclosed subject matter, including a voltage-sensitive rhodopsin domain from Exophiala sideris (EsR (−)) having a D127N Mutation, a HaloTag® protein, a membrane targeting sequence, and an ER export sequence.

SEQ ID NO: 98 is the amino acid sequence of an exemplary voltage indicator provided in accordance with the presently disclosed subject matter, including a voltage-sensitive rhodopsin domain from Moelleriella libera (MIR (−)) having a D121N Mutation, a HaloTag® protein, a membrane targeting sequence, and an ER export sequence.

SEQ ID NO: 99 is the amino acid sequence of an exemplary voltage indicator provided in accordance with the presently disclosed subject matter, including a voltage-sensitive rhodopsin domain from Saitoella complicate (ScR (−)) having a D106N mutation, a HaloTag® protein, a membrane targeting sequence, and an ER export sequence.

SEQ ID NO: 100 is the amino acid sequence of an exemplary voltage indicator provided in accordance with the presently disclosed subject matter, including a voltage-sensitive rhodopsin domain from Rhodotorula graminis (RgR (−)) having a D120N mutation, a HaloTag® protein, a membrane targeting sequence, and an ER export sequence.

SEQ ID NO: 101 is the amino acid sequence of an exemplary voltage indicator provided in accordance with the presently disclosed subject matter, including a voltage-sensitive rhodopsin domain from Rhodotorula toruloides (RtR (−)) having a D109N mutation a HaloTag® protein, a membrane targeting sequence, and an ER export sequence.

SEQ ID NO: 102 is the amino acid sequence of an exemplary voltage indicator provided in accordance with the presently disclosed subject matter, including a voltage-sensitive rhodopsin domain from Gammaproteobacteria (GpR1 (−)) having a D97N mutation, a HaloTag® protein, a membrane targeting sequence, and an ER export sequence.

SEQ ID NO: 103 is the amino acid sequence of an exemplary voltage indicator provided in accordance with the presently disclosed subject matter, including a voltage-sensitive rhodopsin domain from Gammaproteobacteria (GpR2 (−)) having a D98N mutation, a HaloTag® protein, a membrane targeting sequence, and an ER export sequence.

SEQ ID NO: 104 is the amino acid sequence of an exemplary voltage indicator provided in accordance with the presently disclosed subject matter, including a voltage-sensitive rhodopsin domain from Krokinobacter eikastus (KeR1 (−)) having a D88N mutation, a HaloTag® protein, a membrane targeting sequence, and an ER export sequence.

SEQ ID NO: 105 is the amino acid sequence of an exemplary voltage indicator provided in accordance with the presently disclosed subject matter, including a voltage-sensitive rhodopsin domain from Krokinobacter eikastus (KeR2 (−)) having a D116N mutation, a HaloTag® protein, a membrane targeting sequence, and an ER export sequence.

SEQ ID NO: 106 is the amino acid sequence of an exemplary voltage indicator provided in accordance with the presently disclosed subject matter, including a voltage-sensitive rhodopsin domain from Gloeobacter violaceus (GvR (−)) having a D121N mutation, a HaloTag® protein, a membrane targeting sequence, and an ER export sequence.

SEQ ID NO: 107 is the amino acid sequence of an exemplary voltage indicator provided in accordance with the presently disclosed subject matter, including a voltage-sensitive rhodopsin domain from Salinibacter ruber (SrR (−)) having a D96N mutation, a HaloTag® protein, a membrane targeting sequence, and an ER export sequence.

SEQ ID NO: 108 is the amino acid sequence of an exemplary voltage indicator provided in accordance with the presently disclosed subject matter, including a voltage-sensitive rhodopsin domain from Actinomycetes bacterium (AbR) having an E107Q mutation, a HaloTag® protein, a membrane targeting sequence, and an ER export sequence.

SEQ ID NO: 109 is the amino acid sequence of an exemplary voltage indicator provided in accordance with the presently disclosed subject matter, including a voltage-sensitive rhodopsin domain from Halobacterium sp. DLI (HdR), a HaloTag® protein, a membrane targeting sequence, and an ER export sequence.

SEQ ID NO: 110 is the amino acid sequence of an exemplary voltage indicator provided in accordance with the presently disclosed subject matter, including a voltage-sensitive rhodopsin domain from Thermoplasmatales archaeon SG8 (TsR), a HaloTag® protein, a membrane targeting sequence, and an ER export sequence.

SEQ ID NO: 111 is the amino acid sequence of an exemplary voltage indicator provided in accordance with the presently disclosed subject matter, including a voltage-sensitive rhodopsin domain from Emiliania huxleyi (EhR), a HaloTag® protein, a membrane targeting sequence, and an ER export sequence.

SEQ ID NO: 112 is the amino acid sequence of an exemplary voltage indicator provided in accordance with the presently disclosed subject matter, including a voltage-sensitive rhodopsin domain from Heimdallarchaeota archaeon (HaR), a HaloTag® protein, a membrane targeting sequence, and an ER export sequence.

SEQ ID NO: 113 is the amino acid sequence of an exemplary voltage indicator provided in accordance with the presently disclosed subject matter, including a voltage-sensitive rhodopsin domain from Haloarcula argentinensis (Cruxrhodopsin-1) (CR1 (+)) having D94N and E208V mutations, a HaloTag® protein, a membrane targeting sequence, and an ER export sequence.

SEQ ID NO: 114 is the amino acid sequence of an exemplary voltage indicator provided in accordance with the presently disclosed subject matter, including a voltage-sensitive rhodopsin domain from Leptosphaeria maculans (LmR (+)) having D150N and E258V mutations, a HaloTag® protein, a membrane targeting sequence, and an ER export sequence.

SEQ ID NO: 115 is the amino acid sequence of an exemplary voltage indicator provided in accordance with the presently disclosed subject matter, including a voltage-sensitive rhodopsin domain from Podospora anserine (PaR (+)) D147N and E254V mutations, a HaloTag® protein, a membrane targeting sequence, and an ER export sequence.

SEQ ID NO: 116 is the amino acid sequence of an exemplary voltage indicator provided in accordance with the presently disclosed subject matter, including a voltage-sensitive rhodopsin domain from Neurospora crassa (NcR (+)) having E142Q and E251V mutations, a HaloTag® protein, a membrane targeting sequence, and an ER export sequence.

SEQ ID NO: 117 is the amino acid sequence of an exemplary voltage indicator provided in accordance with the presently disclosed subject matter, including a voltage-sensitive rhodopsin domain from Acetabularia acetabulum (Ace1 (+)) having D100N and E206V mutations a HaloTag® protein, a membrane targeting sequence, and an ER export sequence.

SEQ ID NO: 118 is the amino acid sequence of an exemplary voltage indicator provided in accordance with the presently disclosed subject matter, including a voltage-sensitive rhodopsin domain from Chlorella vulgaris (CvR (+)) having D97N and E203V mutations, a HaloTag® protein, a membrane targeting sequence, and an ER export sequence.

SEQ ID NO: 119 is the amino acid sequence of an exemplary voltage indicator provided in accordance with the presently disclosed subject matter, including a voltage-sensitive rhodopsin domain from Klebsormidium nitens (KnR (+)) having D100N and E206V mutations, a HaloTag® protein, a membrane targeting sequence, and an ER export sequence.

SEQ ID NO: 120 is the amino acid sequence of an exemplary voltage indicator provided in accordance with the presently disclosed subject matter, including a voltage-sensitive rhodopsin domain from Taphrina deformans (TdR (+)) having D121N and E227V mutations, a HaloTag® protein, a membrane targeting sequence, and an ER export sequence.

SEQ ID NO: 121 is the amino acid sequence of an exemplary voltage indicator provided in accordance with the presently disclosed subject matter, including a voltage-sensitive rhodopsin domain from Exophiala sideris (EsR (+)) having D127N and E233V mutations, a HaloTag® protein, a membrane targeting sequence, and an ER export sequence.

SEQ ID NO: 122 is the amino acid sequence of an exemplary voltage indicator provided in accordance with the presently disclosed subject matter, including a voltage-sensitive rhodopsin domain from Moelleriella libera (MIR (+)) having D121N and E227V mutations, a HaloTag® protein, a membrane targeting sequence, and an ER export sequence.

SEQ ID NO: 123 is the amino acid sequence of an exemplary voltage indicator provided in accordance with the presently disclosed subject matter, including a voltage-sensitive rhodopsin domain from Saitoella complicate (ScR (+)) having E117Q and E222V mutations, a HaloTag® protein, a membrane targeting sequence, and an ER export sequence.

SEQ ID NO: 124 is the amino acid sequence of an exemplary voltage indicator provided in accordance with the presently disclosed subject matter, including a voltage-sensitive rhodopsin domain from Rhodotorula graminis (RgR (+)) having E131Q and E237V mutations, a HaloTag® protein, a membrane targeting sequence, and an ER export sequence.

SEQ ID NO: 125 is the amino acid sequence of an exemplary voltage indicator provided in accordance with the presently disclosed subject matter, including a voltage-sensitive rhodopsin domain from Rhodotorula toruloides (RtR (+)) having E120Q and E226V mutations, a HaloTag® protein, a membrane targeting sequence, and an ER export sequence.

SEQ ID NO: 126 is the amino acid sequence of an exemplary voltage indicator provided in accordance with the presently disclosed subject matter, including a voltage-sensitive rhodopsin domain from Krokinobacter eikastus (KeR1 (+)) having an E99Q mutation, a HaloTag® protein, a membrane targeting sequence, and an ER export sequence.

SEQ ID NO: 127 is the amino acid sequence of an exemplary voltage indicator provided in accordance with the presently disclosed subject matter, including a voltage-sensitive rhodopsin domain from Krokinobacter eikastus (KeR2 (+)) having a D251N mutation, a HaloTag® protein, a membrane targeting sequence, and an ER export sequence.

SEQ ID NO: 128 is the amino acid sequence of an exemplary voltage indicator provided in accordance with the presently disclosed subject matter, including a voltage-sensitive rhodopsin domain from Gloeobacter violaceus (GvR (+)) having a E132Q mutation, a HaloTag® protein, a membrane targeting sequence, and an ER export sequence.

SEQ ID NO: 129 is the amino acid sequence of an exemplary voltage indicator provided in accordance with the presently disclosed subject matter, including a voltage-sensitive rhodopsin domain from Salinibacter ruber (SrR (+)) having an E107Q mutation, a HaloTag® protein, a membrane targeting sequence, and an ER export sequence.

SEQ ID NO: 130 is the amino acid sequence of an exemplary voltage indicator provided in accordance with the presently disclosed subject matter, including a voltage-sensitive rhodopsin domain from Proteomonas sulcate (PsuCCR (+)) having a D121N mutation, a HaloTag® protein, a membrane targeting sequence, and an ER export sequence.

SEQ ID NO: 131 is the amino acid sequence of an exemplary voltage indicator provided in accordance with the presently disclosed subject matter, including a voltage-sensitive rhodopsin domain from Guillardia theta (GtCCR1 (+)) having a D98N mutation, a HaloTag® protein, a membrane targeting sequence, and an ER export sequence.

SEQ ID NO: 132 is the amino acid sequence of an exemplary voltage indicator provided in accordance with the presently disclosed subject matter, including a voltage-sensitive rhodopsin domain from Guillardia theta (GtCCR2 (+)) having a D98N mutation, a HaloTag® protein, a membrane targeting sequence, and an ER export sequence.

SEQ ID NO: 133 is the amino acid sequence of an exemplary voltage indicator provided in accordance with the presently disclosed subject matter, including a voltage-sensitive rhodopsin domain from Guillardia theta (GtCCR3 (+)) having a D110N mutation, a HaloTag® protein, a membrane targeting sequence, and an ER export sequence.

SEQ ID NO: 134 is the amino acid sequence of an exemplary voltage indicator provided in accordance with the presently disclosed subject matter, including a voltage-sensitive rhodopsin domain from Guillardia theta (GtCCR4 (+)) having a D127N mutation, a HaloTag® protein, a membrane targeting sequence, and an ER export sequence.

SEQ ID NO: 135 is the amino acid sequence of an exemplary voltage indicator provided in accordance with the presently disclosed subject matter, including a voltage-sensitive rhodopsin domain from Guillardia theta (GtCCR5) having a D106N mutation, a HaloTag® protein, a membrane targeting sequence, and an ER export sequence.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter includes voltage indicators, polypeptides, nucleotide molecules, vectors, and methods of use thereof. As disclosed herein, voltage indicators and polypeptides of the presently-disclosed subject matter include a rhodopsin domain. Nucleotide molecules and vectors as disclosed herein comprise an nucleic acid sequence encoding a rhodopsin domain.

As will be appreciated by those of ordinary skill in the art, a rhodopsin domain is a segment of a rhodopsin protein that responds to changes in membrane voltage by altering its conformation. This property can be exploited to convert a rhodopsin into a voltage sensor. In some embodiments, the rhodopsin domain is a microbial rhodopsin domain.

The presently-disclosed subject matter includes a voltage indicator, which comprises a voltage-sensitive rhodopsin domain comprising an amino acid sequence selected from the group consisting of SEQ ID NO: 1-30, including a mutation to replace a charged canonical amino acid residue with a neutral residue at one, two, or three residues within a pump or channel core motif of the rhodopsin domain. The voltage indicator also comprises a capture protein that covalently or noncovalently binds a fluorescent element that is a fluorescent protein, or a fluorescent dye. The capture protein is provided together with the voltage-sensitive rhodopsin domain in a fusion protein.

As used herein, the term “charged canonical amino acid residue” refers to an amino acid residue selected from the twenty (20) canonical amino acids, and having a side chain that carries a charge at physiological pH (about 7.4). For example, aspartic acid (Asp, D) and glutamic acid (Glu, E) have a negatively charged carboxylate group, lysine (Lys, K) has a positively charged amino group, and arginine (Arg, R) has a positively charged guanidinium group.

As used herein, the term “neutral residue” refers to an amino acid residue having a side chain that does not carry a charge at physiological pH (about 7.4). For example, alanine (Ala, A), asparagine (Asn, N), cysteine (Cys, C), glutamine (Gln, Q), glycine (Gly, G), isoleucine (Ile, I), leucine (Leu, L), methionine (Met, M), phenylalanine (Phe, F), proline (Pro, P), serine (Ser, S), threonine (Thr, T), tryptophan (Trp, W), tyrosine (Tyr, Y), and valine (Val, V) have side chains that are either nonpolar or polar but uncharged at physiological pH. Additional noncanonical amino acid are also known in the art, which have side chains that are either nonpolar or polar but uncharged at physiological pH.

The term “pump or channel core motif” when used in connection with a rhodopsin domain refers to a specific cluster of about 5-6 amino acid residues located within the rhodopsin domain that is important for the function of the pump or channel. This motif includes key residues such as the counterion position, protein donor position, proton release position(s), sodium pump position(s), and cation channel position(s), which collectively contribute to fundamental mechanisms like ion transport, proton transfer, or gating within the protein. As will be appreciated by those of ordinary skill in the art, the specific residues of the pump or channel core motif can be determined using one or more techniques well-known in the art, such as, for example, sequence analysis (e.g., sequence alignment, homology modeling, etc.), crystallography, cryo-electron microscopy, mutagenesis studies, spectroscopic methods (e.g., UV, FTIR, NMR etc.), molecular dynamics simulations, functional assays, and protonation studies.

The term “counterion position” refers to a specific location of an amino acid residue in the pump or channel core motif of the rhodopsin domain that stabilizes the positively charged Schiff base within the rhodopsin's chromophore binding site. As will be appreciated by those of ordinary skill in the art, the specific residue serving as the counterion can vary between different rhodopsin domains, but can be determined using one or more techniques well-known in the art, such as, for example, sequence analysis (e.g., sequence alignment, homology modeling, etc.), crystallography, cryo-electron microscopy, mutagenesis studies, spectroscopic methods (e.g., UV, FTIR, NMR etc.), molecular dynamics simulations, functional assays, and protonation studies. By way of example, a counterion position for rhodopsin domain from Klebsormidium nitens (SEQ ID NO: 1) is Asp89. For another example, a counterion position for the rhodopsin domain from Podospora anserine (SEQ ID NO: 2) is Asp136.

The term “proton donor position” refers to a specific location of an amino acid residue in the pump or channel core motif of the rhodopsin domain where a proton (H⁺ ion) is donated during a chemical reaction. In the context of rhodopsins, this position is important for processes like proton transfer, which is important for the protein's function in ion transport or gating mechanisms. As will be appreciated by those of ordinary skill in the art, the specific residue of the protein donor position can be determined using one or more techniques well-known in the art, such as, for example, sequence analysis (e.g., sequence alignment, homology modeling, etc.), crystallography, cryo-electron microscopy, mutagenesis studies, spectroscopic methods (e.g., UV, FTIR, NMR etc.), molecular dynamics simulations, functional assays, and protonation studies.

The term “protein release position” refers to specific location(s) of an amino acid residue(s) in the pump or channel core motif of the rhodopsin domain where protons (H⁺ ions) are released during the protein's functional cycle. These positions are crucial for processes such as proton transfer and ion transport, which are important for the activity of rhodopsins. In rhodopsins, the proton release positions are typically involved in the final steps of the proton transfer pathway, where protons are released to the extracellular environment or another part of the protein structure, facilitating the overall function of the pump or channel. As will be appreciated by those of ordinary skill in the art, the specific residues of the proton release position can be determined using one or more techniques well-known in the art, such as, for example, sequence analysis (e.g., sequence alignment, homology modeling, etc.), crystallography, cryo-electron microscopy, mutagenesis studies, spectroscopic methods (e.g., UV, FTIR, NMR etc.), molecular dynamics simulations, functional assays, and protonation studies.

The term “sodium pump position(s)” refers to specific location(s) of amino acid residue(s) that are important for the binding and transport of sodium ions (Na⁺). These residues are typically located within the transmembrane domain of the rhodopsin protein. The precise positioning and interaction of these amino acids are important for the protein's function as a sodium pump, ensuring the correct translocation of Na+ ions across the cell membrane. As will be appreciated by those of ordinary skill in the art, the specific residues of the sodium pump position(s) can be determined using one or more techniques well-known in the art, such as, for example, sequence analysis (e.g., sequence alignment, homology modeling, etc.), crystallography, cryo-electron microscopy, mutagenesis studies, spectroscopic methods (e.g., UV, FTIR, NMR etc.), molecular dynamics simulations, functional assays, and protonation studies.

The term “cation channel position(s)” refers to specific location(s) of amino acid residue(s) that are important for the binding and transport of cations (such as Na⁺ or K⁺) through the channel. These residues are typically located within the transmembrane domains of the rhodopsin protein. As will be appreciated by those of ordinary skill in the art, the specific residues of the cation channel position(s) can be determined using one or more techniques well-known in the art, such as, for example, sequence analysis (e.g., sequence alignment, homology modeling, etc.), crystallography, cryo-electron microscopy, mutagenesis studies, spectroscopic methods (e.g., UV, FTIR, NMR etc.), molecular dynamics simulations, functional assays, and protonation studies.

As used herein, the term “mutation” means a change introduced in a nucleic acid or amino acid sequence, such as in the case of an amino acid at a particular residue in an amino acid sequence being replaced with a different amino acid residue. Embodiments of the voltage indicator and polypeptides of the presently disclosed subject matter include a mutation at one, two, or three residues within a pump or channel core motif of the rhodopsin domain comprising an amino acid sequence selected from the group consisting of SEQ ID NO: 1-30.

In some embodiments, when the voltage-sensitive rhodopsin domain is from Klebsormidium nitens (KnR), the mutation can be D89X, D100X, and/or E206X where X is a neutral residue, with reference to SEQ ID NO: 1. For example, X can be alanine (A), asparagine (N), cysteine (C), glutamine (Q), glycine (G), isoleucine (I), leucine (L), methionine (M), phenylalanine (F), proline (P), serine(S), threonine (T), tryptophan (W), tyrosine (Y), or valine (V).

In some embodiments, when the voltage-sensitive rhodopsin domain is from Podospora anserine (PaR), the mutation can be D136X, D147X, and/or E254X, where X is a neutral residue, with reference to SEQ ID NO: 2. For example, X can be A, N, C, Q, G, I, L, M, F, P, S, T, W, Y, or V.

In some embodiments, when the voltage-sensitive rhodopsin domain is from Haloarcula argentinensis (Cruxrhodopsin-1) (CR1), the mutation can be D83X, D94X, and/or E208X where X is a neutral residue, with reference to SEQ ID NO: 3. For example, X can be A, N, C, Q, G, I, L, M, F, P, S, T, W, Y, or V.

In some embodiments, when the voltage-sensitive rhodopsin domain is from Leptosphaeria maculans (LmR), the mutation can be D139X, D150X, and/or E258X where X is a neutral residue, with reference to SEQ ID NO: 4. For example, X can be A, N, C, Q, G, I, L, M, F, P, S, T, W, Y, or V.

In some embodiments, when the voltage-sensitive rhodopsin domain is from Neurospora crassa (NcR), the mutation can be D131X, E142X, and/or E251X where X is a neutral residue, with reference to SEQ ID NO: 5. For example, X can be A, N, C, Q, G, I, L, M, F, P, S, T, W, Y, or V.

In some embodiments, when the voltage-sensitive rhodopsin domain is from Acetabularia acetabulum (Ace1), the mutation can be D89X, D100X, and/or E206X where X is a neutral residue, with reference to SEQ ID NO: 6. For example, X can be A, N, C, Q, G, I, L, M, F, P, S, T, W, Y, or V.

In some embodiments, when the voltage-sensitive rhodopsin domain is from Chlorella vulgaris (CvR), the mutation can be D86X, D97X, and/or E203X where X is a neutral residue, with reference to SEQ ID NO: 7. For example, X can be A, N, C, Q, G, I, L, M, F, P, S, T, W, Y, or V.

In some embodiments, when the voltage-sensitive rhodopsin domain is from Taphrina deformans (TdR), the mutation can be D110X, D121X, and/or E227X where X is a neutral residue, with reference to SEQ ID NO: 8. For example, X can be A, N, C, Q, G, I, L, M, F, P, S, T, W, Y, or V.

In some embodiments, when the voltage-sensitive rhodopsin domain is from Exophiala sideris (EsR), the mutation can be D116X, D127X, and/or E233X where X is a neutral residue, with reference to SEQ ID NO: 9. For example, X can be A, N, C, Q, G, I, L, M, F, P, S, T, W, Y, or V.

In some embodiments, when the voltage-sensitive rhodopsin domain is from Moelleriella libera (MIR), the mutation can be D110X, D121X, and/or E227X where X is a neutral residue, with reference to SEQ ID NO: 10. For example, X can be A, N, C, Q, G, I, L, M, F, P, S, T, W, Y, or V.

In some embodiments, when the voltage-sensitive rhodopsin domain is from Saitoella complicate (ScR), the mutation can be D106X, E117X, and/or E222X where X is a neutral residue, with reference to SEQ ID NO: 11. For example, X can be A, N, C, Q, G, I, L, M, F, P, S, T, W, Y, or V.

In some embodiments, when the voltage-sensitive rhodopsin domain is from Rhodotorula graminis (RgR), the mutation can be D120X, E131X, and/or E237X where X is a neutral residue, with reference to SEQ ID NO: 12. For example, X can be A, N, C, Q, G, I, L, M, F, P, S, T, W, Y, or V.

In some embodiments, when the voltage-sensitive rhodopsin domain is from Rhodotorula toruloides (RtR), the mutation can be D109X, E120X, and/or E226X where X is a neutral residue, with reference to SEQ ID NO: 13. For example, X can be A, N, C, Q, G, I, L, M, F, P, S, T, W, Y, or V.

In some embodiments, when the voltage-sensitive rhodopsin domain is from Krokinobacter eikastus (KeR1), the mutation can be D88X and/or E99X where X is a neutral residue, with reference to SEQ ID NO: 14. For example, X can be A, N, C, Q, G, I, L, M, F, P, S, T, W, Y, or V.

In some embodiments, when the voltage-sensitive rhodopsin domain is from Krokinobacter eikastus (KeR2), the mutation can be D116X, D251X where X is a neutral residue, with reference to SEQ ID NO: 15. For example, X can be A, N, C, Q, G, I, L, M, F, P, S, T, W, Y, or V.

In some embodiments, when the voltage-sensitive rhodopsin domain is from Gloeobacter violaceus (GvR), the mutation can be D121X and/or E132X where X is a neutral residue, with reference to SEQ ID NO: 16. For example, X can be A, N, C, Q, G, I, L, M, F, P, S, T, W, Y, or V.

In some embodiments, when the voltage-sensitive rhodopsin domain is from Salinibacter ruber (SrR), the mutation can be D96X and/or E107X where X is a neutral residue, with reference to SEQ ID NO: 17. For example, X can be A, N, C, Q, G, I, L, M, F, P, S, T, W, Y, or V.

In some embodiments, when the voltage-sensitive rhodopsin domain is from Gammaproteobacteria (GpR1), the mutation can be D97X and/or E108X where X is a neutral residue, with reference to SEQ ID NO: 18. For example, X can be A, N, C, Q, G, I, L, M, F, P, S, T, W, Y, or V.

In some embodiments, when the voltage-sensitive rhodopsin domain is from Gammaproteobacteria (GpR2), the mutation can be D98X and/or E109X where X is a neutral residue, with reference to SEQ ID NO: 19. For example, X can be A, N, C, Q, G, I, L, M, F, P, S, T, W, Y, or V.

In some embodiments, when the voltage-sensitive rhodopsin domain is from Proteomonas sulcate (PsuCCR), the mutation can be D121X where X is a neutral residue, with reference to SEQ ID NO: 20. For example, X can be A, N, C, Q, G, I, L, M, F, P, S, T, W, Y, or V.

In some embodiments, when the voltage-sensitive rhodopsin domain is from Guillardia theta (GtCCR1), the mutation can be D98X where X is a neutral residue, with reference to SEQ ID NO: 21. For example, X can be A, N, C, Q, G, I, L, M, F, P, S, T, W, Y, or V.

In some embodiments, when the voltage-sensitive rhodopsin domain is from Guillardia theta (GtCCR2), the mutation can be D98X where X is a neutral residue, with reference to SEQ ID NO: 22. For example, X can be A, N, C, Q, G, I, L, M, F, P, S, T, W, Y, or V.

In some embodiments, when the voltage-sensitive rhodopsin domain is from Guillardia theta (GtCCR3), the mutation can be D110X where X is a neutral residue, with reference to SEQ ID NO: 23. For example, X can be A, N, C, Q, G, I, L, M, F, P, S, T, W, Y, or V.

In some embodiments, when the voltage-sensitive rhodopsin domain is from Guillardia theta (GtCCR4), the mutation can be D127X where X is a neutral residue, with reference to SEQ ID NO: 24. For example, X can be A, N, C, Q, G, I, L, M, F, P, S, T, W, Y, or V.

In some embodiments, when the voltage-sensitive rhodopsin domain is from Guillardia theta (GtCCR5), the mutation can be D106X where X is a neutral residue, with reference to SEQ ID NO: 25. For example, X can be A, N, C, Q, G, I, L, M, F, P, S, T, W, Y, or V.

In some embodiments, when the voltage-sensitive rhodopsin domain is from Emiliania huxleyi (EhR), the mutation can be D88X where X is a neutral residue, with reference to SEQ ID NO: 26. For example, X can be A, N, C, Q, G, I, L, M, F, P, S, T, W, Y, or V.

In some embodiments, when the voltage-sensitive rhodopsin domain is from Actinomycetes bacterium (AbR), the mutation can be E107X where X is a neutral residue, with reference to SEQ ID NO: 27. For example, X can be A, N, C, Q, G, I, L, M, F, P, S, T, W, Y, or V.

In some embodiments, when the voltage-sensitive rhodopsin domain is from Heimdallarchaeota archaeon (HaR), the mutation can be N108X where X is a neutral residue, with reference to SEQ ID NO: 28. For example, X can be A, C, Q, G, I, L, M, F, P, S, T, W, Y, or V.

In some embodiments, when the voltage-sensitive rhodopsin domain is from Halobacterium sp. DLI (HdR), the mutation can be D107X where X is a neutral residue, with reference to SEQ ID NO: 29. For example, X can be A, N, C, Q, G, I, L, M, F, P, S, T, W, Y, or V.

In some embodiments, when the voltage-sensitive rhodopsin domain is from Thermoplasmatales archaeon SG8 (TsR), the mutation can be D128 where X is a neutral residue, with reference to SEQ ID NO: 30. For example, X can be A, N, C, Q, G, I, L, M, F, P, S, T, W, Y, or V.

In some embodiments, the voltage indicator or polypeptide of the presently-disclosed subject matter comprises an amino acid sequence selected from the group consisting of SEQ ID NO: 31-74.

The voltage indicator or polypeptide of the presently-disclosed subject matter further comprises a capture protein. The term “capture protein” refers to a protein that binds with affinity and specificity to a particular ligand. Examples of capture proteins include self-labeling proteins that bind to their particular ligands, and avidin or streptavidin that binds to biotin. In some embodiments of the presently-disclosed voltage indicator, the capture protein is positioned at the c-terminal end of the voltage-sensitive rhodopsin domain. In some embodiments of the presently-disclosed voltage indicator, the capture protein is fused to the c-terminus of the voltage-sensitive rhodopsin domain following the first 7-transmembrane helices of the rhodopsin.

In some embodiments, the capture protein is a self-labeling protein or avidin. A “self-labeling protein” is a protein that can covalently attach to a specific chemical substrate, referred to as a ligand or self-labeling protein ligand. A “self-labeling protein ligand” is a chemical compound that specifically binds to a self-labeling protein, forming a stable covalent bond. These ligands can be linked to chemicals or functional groups. Together, the self-labeling protein and the self-labeling protein ligand provide a system for facilitating the specific attachment of a compound or functional group to a protein within a living cell or in vitro. The term “self-labeling” indicates that the protein is capable of catalyzing the attachment to the compound without the need for additional enzymes or co-factors.

A self-labeling protein/ligand system includes the self-labeling protein (SLP) (sometimes referred to in the art as a self-labeling protein tag) and the SLP ligand. The SLP and the SLP ligand form a specific bond. In this regard, when the SLP ligand is attached to a compound or functional group, the SLP forms a bond with the compound or functional group via the SLP ligand. This bond formation ensures a stable and irreversible attachment of the compound or functional group to the protein, allowing for various applications such as visualization, purification, and interaction studies.

Examples of SLPs and their SLP ligands will be known to those of ordinary skill in the art. One such example is HaloTag®, which is a modified bacterial enzyme that binds covalently to synthetic ligands containing a chloroalkane (CLA) moiety. Another example is SNAP-tag®, which is derived from the human DNA repair protein 06-alkylguanine-DNA alkyltransferase (AGT), and binds covalently to derivatives of its substrate 06-benzylguanine (BG). Another example is CLIP-tag®, which is similar to SNAP-tag®, but it binds to 02-benzylcytosine (BC) derivatives, allowing for orthogonal labeling in combination with SNAP-tag®. Another example is TMP-tag®, which is a self-labeling protein that binds to trimethoprim (TMP) derivatives. Another example is a tetracysteine tag, which is a peptide sequence that binds to biarsenical dyes like fluorescein arsenical helix binder (FLASH) and Resorufin Arsenical Helix binder (ReAsH), which are useful for fluorescent labeling. Another example is BLac-tag, which is a self-labeling protein derived from B-lactamase, which is often used with a ligand that is a β-lactam antibiotic, such as cephalosporin or penicillin derivatives. In some embodiments, the SLP is a HaloTag® comprising the sequence of SEQ ID NO: 75.

Another example is avidin-biotin, in which avidin (or streptavidin) can be provided as the capture protein, which binds with high affinity and specificity to the ligand biotin, which is a vitamin (B7) that can be covalently attached to proteins, nucleic acids, or other compounds or functional groups.

In some embodiments, the voltage indicator further comprises a 1, 2, 3, 4, 5, 6, 7, 8, 9, or 10 amino acid linker between the voltage-sensitive rhodopsin domain and the capture protein.

In some embodiments, the voltage indicator further comprises a fluorescent element. The term “fluorescent element” refers to a component that emits light upon excitation, and is inclusive fluorescent dyes and fluorescent proteins. As will be appreciated by one of ordinary skill in the art upon study of this document, the fluorescent element is used to detect changes in membrane voltage (e.g., in neurons) by measuring variations in fluorescence intensity, allowing for the optical recording of voltage changes in neurons using fluorescence imaging.

As will be understood by one of ordinary skill in the art, a fluorescent dye is a synthetic molecule that emits light upon excitation. In embodiments of the presently disclosed subject matter that make use of a fluorescent dye, a ligand of the capture protein is covalently or non-covalently attached to the fluorescent dye, such that when the capture protein and its ligand bind, the fluorescent dye becomes attached to the other components of the voltage indicator.

As will be appreciated, there are many fluorescent dyes known in the art, and which can be selected for use in the context of the presently-disclosed subject matter. When selecting a fluorescent dye for use one would consider, for example, brightness for sufficient signal intensity, photostability to maintain fluorescence over time, spectral properties to match the excitation and emission wavelengths with available microscopy equipment and to avoid overlap with other fluorescent markers, compatibility with the biological system to avoid toxicity and ensure proper cellular localization, and response time to accurately track rapid voltage changes and a high signal-to-noise ratio to distinguish the signal from background fluorescence. Accordingly there are many different fluorescent dyes known in the art that can be selected by one of ordinary skill in the art with consideration to these and other criteria known in the art.

A few examples from among the many fluorescent dyes that could be used are azetidine-containing fluorescent dyes, including Janelia Fluor® dyes, such as JF525, JF549, JF525, JF646, and JF608. In addition to azetidine-containing dyes, several other classes of fluorescent dyes are commonly used in the context of genetically encoded voltage indicators (GEVIs). Rhodamines, such as tetramethylrhodamine (TMR) and rhodamine B, are known for their high brightness and photostability. Cyanine dyes, including Cy3, Cy5, and Cy7, offer a wide range of excitation and emission wavelengths, which is beneficial for multiplexing with other fluorescent markers. BODIPY dyes, like BODIPY-FL and BODIPY-TMR, are appreciated for their high fluorescence quantum yields and stability. Fluoresceins, such as fluorescein and its derivatives like Oregon Green and Alexa Fluor 488, are widely used due to their high fluorescence intensity and compatibility with various biological systems. Alexa Fluor dyes, including Alexa Fluor 488, 555, and 647, are known for their high photostability and brightness. Atto dyes, such as Atto 488, Atto 550, and Atto 647N, provide high photostability and brightness, making them suitable for super-resolution microscopy and other advanced imaging techniques.

As will be understood by one of ordinary skill in the art, a fluorescent protein is a genetically encoded protein that emits light upon excitation. In embodiments of the presently disclosed subject matter that make use of a fluorescent protein, a nucleic acid sequence encoding a amino acid sequence comprising the rhodopsin domain and the capture protein is expressed together with a nucleic acid sequence encoding an amino acid sequence of the fluorescent protein, thereby producing a fusion protein such that the fluorescent protein becomes attached to the other components of the voltage indicator.

As will be appreciated, there are many fluorescent proteins known in the art, and which can be selected for use in the context of the presently-disclosed subject matter. When selecting a fluorescent protein for use one would consider, for example, brightness, to ensure a strong signal; photostability, to maintain fluorescence over time; spectral properties, to match the available microscopy equipment and avoid overlap with other fluorescent markers; maturation time of the protein, as it needs to fold and mature to be functional; fluorescence stability at a desired range of pH values, especially those found in the cellular environment; compatibility with the biological system to avoid toxicity and ensure normal cellular function; ability to maintain its fluorescence when fused to other proteins and avoid interfering with the function of the protein it is fused to. For many applications, especially in fusion proteins, it is useful for the fluorescent protein to be monomeric to avoid aggregation that can interfere with cellular processes. Accordingly there are many different fluorescent protein known in the art that can be selected by one of ordinary skill in the art with consideration to these and other criteria known in the art.

A few examples from among the many fluorescent proteins that could be used are Green Fluorescent Protein (GFP) and its derivatives, such as Enhanced GFP (EGFP) and Superfolder GFP (sfGFP), which are widely used for their robust fluorescence and ease of use; mCherry and mRuby are red fluorescent proteins that offer good photostability and are often used in combination with green fluorescent proteins for multiplexing; Cyan Fluorescent Protein (CFP) and Yellow Fluorescent Protein (YFP) are also useful choices, particularly in Förster Resonance Energy Transfer (FRET) applications; mNeonGreen is known for its high brightness and photostability; Cyan-Excitable Orange Fluorescent Protein (CyOFP), which is excitable by cyan light and emits orange fluorescence; Monomeric Orange Fluorescent Protein (mOrange), which provides bright orange fluorescence; and m Venus, a variant of YFP that offers desirable brightness and maturation speed. In some embodiments, the fluorescent protein comprising an amino acid sequence selected from the group consisting of SEQ ID NO: 76-78.

In some embodiments, the voltage indicator or polypeptide further comprises a targeting sequence. A targeting sequence is a short stretch of amino acids within a protein that directs the protein to a desired location within the cell. A soma targeting sequence, for example, is a short amino acid sequence within a protein that directs the protein to the soma, or cell body, of a neuron. For another example, a membrane targeting sequence is a short amino acid sequence within a protein that directs the protein to a specific membrane within the cell. These sequences are recognized by cellular machinery, which facilitates the transport and insertion of the protein into the appropriate membrane, such as the plasma membrane, endoplasmic reticulum, mitochondria, or other organelles. The length of the targeting sequence typically ranges from about 5 to 30 residues. These sequences are long enough to provide the necessary information for directing the protein to its correct cellular location but short enough to be efficiently processed by the cell.

In some embodiments, the voltage indicator or polypeptide further comprises an endoplasmic reticulum (ER) export sequence. An endoplasmic reticulum (ER) export sequence is a specific amino acid sequence within a protein that signals for its transport from the ER to the Golgi apparatus.

In some embodiments, the voltage indicator or polypeptide further comprises an amino acid sequence selected from the group consisting of SEQ ID NO: 79-82.

In some embodiments, the voltage indicator or polypeptide comprises an amino acid sequence selected from the group consisting of SEQ ID NO: 83-135.

The presently-disclosed subject matter further includes nucleotide molecules. In some embodiments, the nucleotide molecule comprises a nucleotide encoding an amino acid sequence selected from the group consisting of SEQ ID NO: 1-30, including a mutation to replace a charged canonical amino acid residue with a neutral residue at a residue within a pump or channel core motif of the rhodopsin domain. In some embodiments, the nucleotide molecule comprises a nucleotide encoding an amino acid selected from the group consisting of SEQ ID NO: 31-74.

In some embodiments, the nucleotide molecule further comprises a nucleotide encoding an amino acid sequence for a capture protein. In some embodiments, the nucleotide molecule further comprises a nucleotide encoding an amino acid sequence of a HaloTag® comprising the sequence of SEQ ID NO: 75.

In some embodiments, the nucleotide molecule further comprises a nucleotide encoding an amino acid sequence for a fluorescent protein. In some embodiments, the nucleotide molecule comprises a nucleotide encoding an amino acid sequence for a fluorescent protein selected from the group consisting of SEQ ID NO: 76-78.

In some embodiments, the nucleotide molecule further comprises a nucleotide encoding an amino acid sequence for a targeting sequence and/or an endoplasmic reticulum (ER) export sequence. In some embodiments, the nucleotide molecule further comprises a nucleotide encoding an amino acid sequence selected from the group consisting of SEQ ID NO: 79-82.

In some embodiments, the nucleotide molecule further comprises a nucleotide encoding an amino acid sequence selected from the group consisting of SEQ ID NO: 83-135.

The presently-disclosed subject matter further includes a vector comprising a nucleotide molecule as disclosed herein.

The presently-disclosed subject matter further includes a method of measuring voltage, which comprises (i) delivering to a cell a nucleotide encoding an amino acid molecule comprising a voltage-sensitive rhodopsin domain comprising an amino acid sequence selected from the group consisting of SEQ ID NO: 1-30, including a mutation to replace a charged canonical amino acid residue with a neutral residue at one, two, or three residues within a pump or channel core motif of the rhodopsin domain; and a capture protein that covalently or noncovalently binds a fluorescent element that is a fluorescent protein, or a fluorescent dye; wherein the capture protein is provided together with the voltage-sensitive rhodopsin domain in a fusion protein; and (ii) delivering to the cell a fluorescent element that is a fluorescent protein or a fluorescent dye, wherein when the fluorescent element is a fluorescent protein, it is provided together with the capture protein and the voltage-sensitive rhodopsin domain in a fusion protein; and when the fluorescent element is a fluorescent dye, it is attached to a ligand of the capture protein; and (iii) determining changes in fluorescence of the fluorescent element, as an measurement of voltage changes in the cell, wherein an increase in membrane potential lead to an increase in fluorescence.

In some embodiments of the method, the cell is a neuron. In some embodiments, the method further comprises observing changes in fluorescence with a microscope. In some embodiments, the microscope is a two-photon microscope. In some embodiments, the method is used for deep tissue imaging. Embodiments of the method make use of the various voltage indicators, polypeptides, nucleotide molecules, and vectors as disclosed herein.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUBMB Joint Commission on Biochemical Nomenclature (See, iubmb.qmul.ac.uk/).

Although any methods, devices, and materials similar or equivalent to those described herein can be used in the practice or testing of the presently-disclosed subject matter, representative methods, devices, and materials are described herein.

In certain instances, nucleotides and polypeptides disclosed herein are included in publicly-available databases, such as ADDGENE®. Information including sequences and other information related to such nucleotides and polypeptides included in such publicly-available databases are expressly incorporated by reference. Unless otherwise indicated or apparent the references to such publicly-available databases are references to the most recent version of the database as of the filing date of this Application.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

The presently-disclosed subject matter is further illustrated by the following specific but non-limiting examples. The following examples may include compilations of data that are representative of data gathered at various times during the course of development and experimentation related to the present invention.

### EXAMPLES

### Example 1—Exploration of Diverse Microbial Rhodopsins as Voltage Indicators

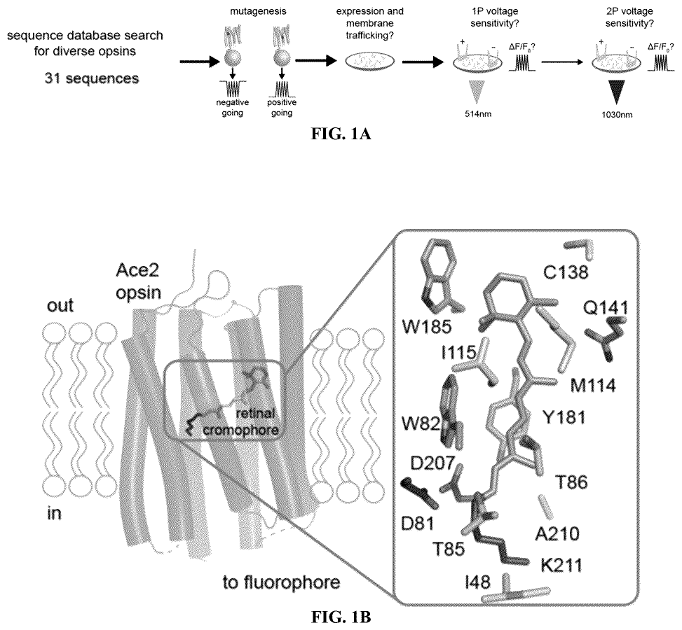

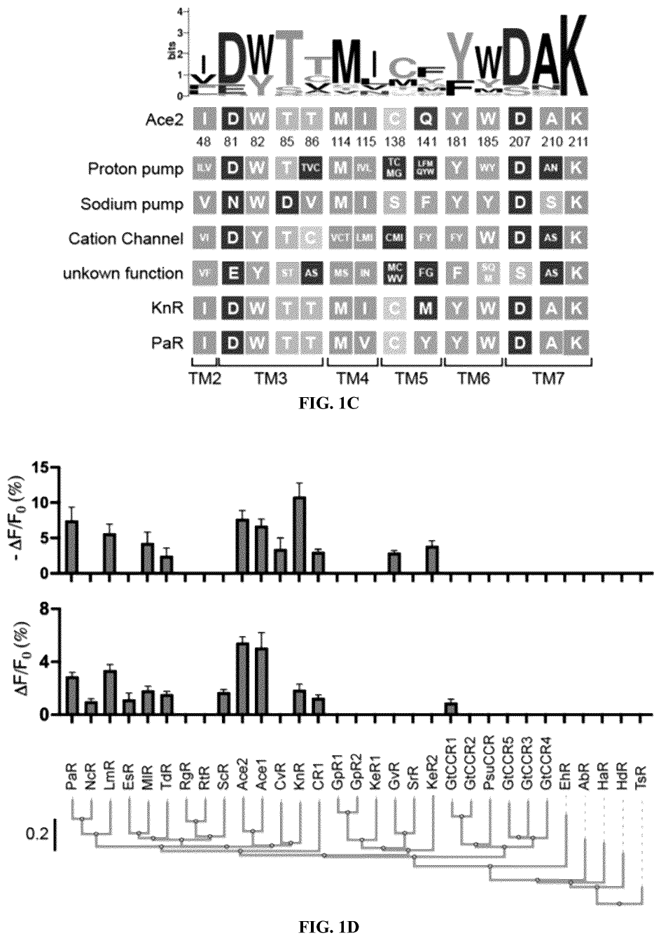

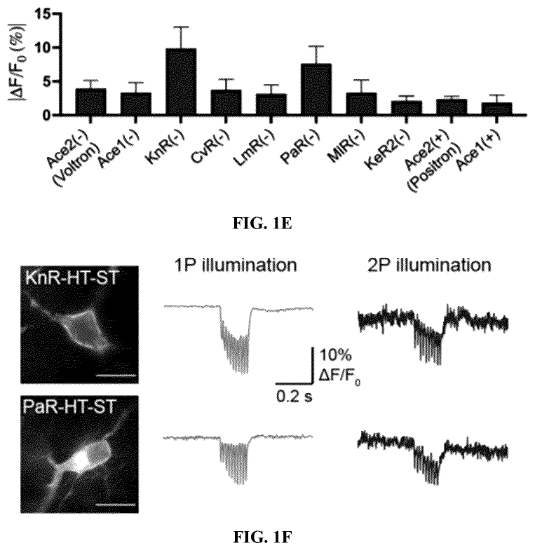

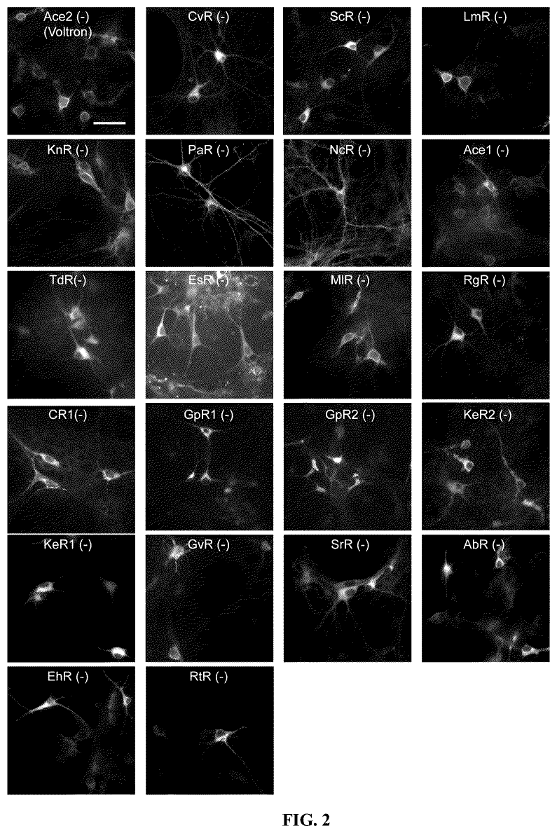

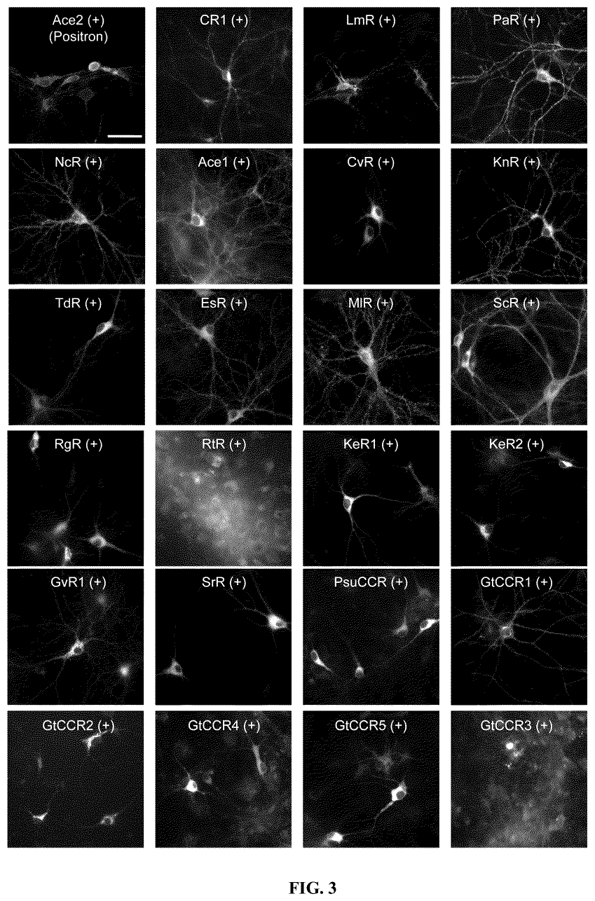

A diverse set of 31 microbial rhodopsin proteins were studied, chosen to sample multiple functional rhodopsin classes and to broadly explore sequence space, as voltage indicators to identify improved GEVIs for 2P imaging (FIG. 1A; Table 1 and Table 2). These microbial opsins are cation channels, proton or sodium pumps, or have no characterized function (FIG. 1B-1C). For each opsin, mutations were introduced to block pump or channel activity. For proton pumps, the counterion or proton donor residues were mutated from negatively charged to neutral^{6,19-21}. These mutations have been shown to block photocurrent and generate either negative-going or positive-going GEVIs^{1,8}. For sodium pumps, similar mutations were introduced to block photocurrent and generate GEVIs. For cation channels, the analogous site to the proton donor position were mutated to a neutral residue to block channel activity22 and potentially generate positive-going GEVIs. Each of the modified rhodopsin sequences were fused to HaloTag (HT) to image the FRET response of a bound fluorescent dye-HaloTag ligand (HTL) 3, expressed them in primary rat hippocampal neurons in culture from a strong promoter and observed their expression and membrane trafficking after labeling with JF₅₂₅-HTL (FIGS. 2 and 3).

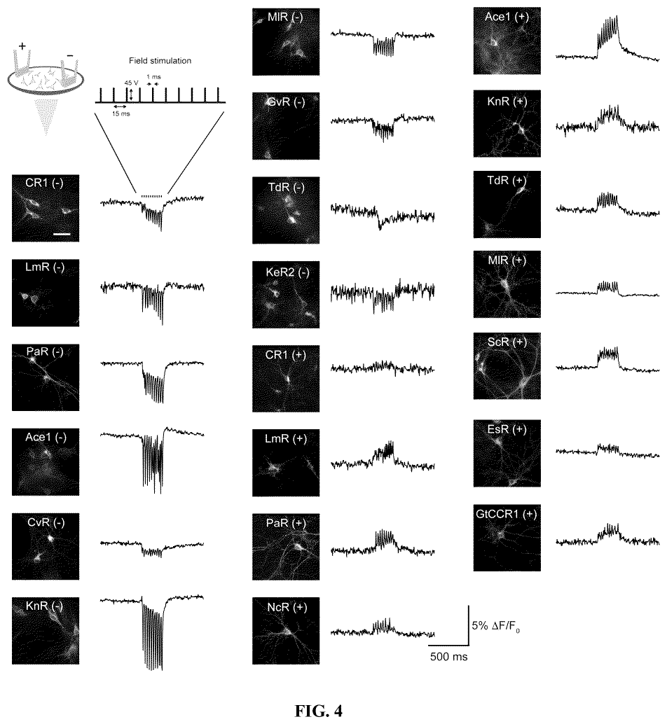

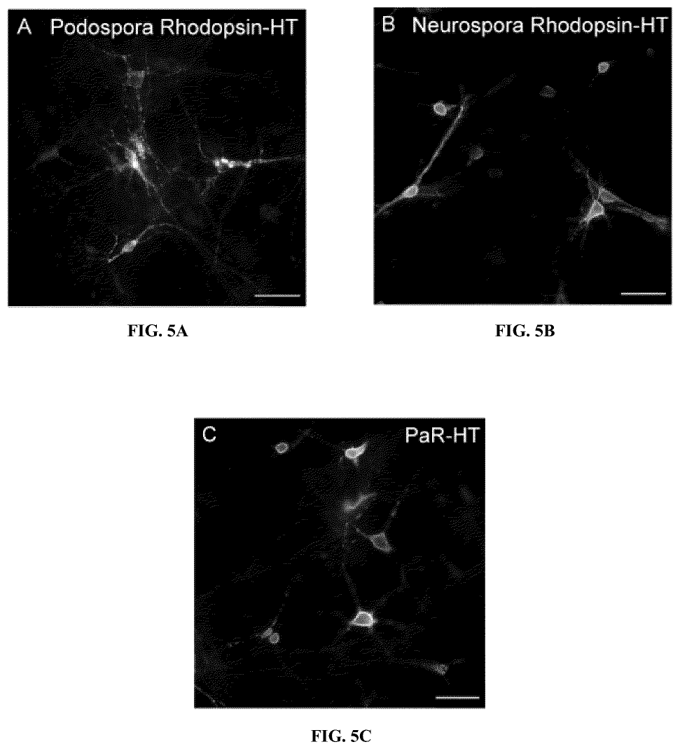

For opsin variants that showed reasonable expression in neurons, 1P fluorescence response to membrane voltage changes was tested by inducing a train of 10 action potentials with a field electrode (FIG. 1D and FIG. 4). For proteins that showed strong 1P fluorescence responses to neuron stimulation, their response to the same stimulation was measured on a custom high-speed 2P microscope¹⁸ (FIG. 1E). Two rhodopsin proteins were identified, one from the alga Klebsormidium nitens (KnR) and another from a fungus, Podospora anserine (PaR), that showed significant 1P sensitivity and retained their ability to report action potentials with 2P illumination under the conditions of the screen (FIG. 1F). PaR required grafting 39 residues from the N-terminus of the rhodopsin from Neurospora crassa in place of the first 44 residues, which displayed dramatically better membrane trafficking (FIG. 5A-5C). KnR and PaR have <35% sequence identity when compared to any reported opsin-based voltage sensor (FIG. 6).

TABLE 1
Rhodopsin domain sequences used for fusion with HaloTag based on sequence alignment
with the 7 transmembrane domain of Ace2 rhodopsin

| Organism | |
|---|---|
| (abbreviation) | Sequence |
| Klebsormidium | MVYSHADAAGWTLYWITYGIMAVTALIFFAMSLRRPIQQRSHHYTSFLIVAIASLAYYAM |
| nitens | ASQGGNTRIRVYQGPADSYRQIFWARYVDWFFTTPLLLLDLVLLSNLSKLRIAAIMVADI |
| (KnR) | FMILTGLFGAVEARSNKWGWFVFGCIFMLYIFYELLVNVRKGAYARGGQHGMLYSVLLVW |
| | LLILWVQYPVVWGLAEGSSTVSSDTEIAWYAALDICAKCVFGFILLLGIESIDRKR |
| | (SEQ ID NO: 1) |
| Podospora | MIHHDQVAEMLYGYAGAAAAKPTHTDSPGPIPTVIPTPPQFQEIGETGHRTLWVVFALMV |
| anserina | LSSGFFAFMSWNVPISKRLYHVITTLITITASLSYFAMASGHVTSFSCTPAKDHHKHVPD |
| (PaR) | VGYTECRQVFWGRYVDWAITTPLLLLDLSLLAGIDGAHTLMAVIADVIMVLSGLFASQGE |
| | TATQRWGWYAIGCVSYLFVIWHVALHGARTVTAKGRGVTRLFSSLALFTFVLWTAYPIVW |
| | GIADGAHRTTVDTEILIYAVLDILAKPVFGLWLLFSHRSLAETN |
| | (SEQ ID NO: 2) |
| Haloarcula | MPEPGSEAIWLWLGTAGMFLGMLYFIARGWGETDSRRQKFYIATILITAIAFVNYLAMAL |
| argentinensis | GFGLTIVEFAGEEHPIYWARYSDWLFTTPLLLYDLGLLAGADRNTITSLVSLDVLMIGTG |
| (Cruxrhodopsin-1) | LVATLSPGSGVLSAGAERLVWWGISTAFLLVLLYFLFSSLSGRVADLPSDTRSTFKTLRN |
| (CR1) | LVTVVWLVYPVWWLIGTEGIGLVGIGIETAGFMVIDLTAKVGFGIILLRSHGVLDGAA |
| | (SEQ ID NO: 3) |
| Leptosphaeria | MIVDQFEEVLMKTSQLFPLPTATQSAQPTHVAPVPTVLPDTPIYETVGDSGSKTLWVVFV |
| maculans | LMLIASAAFTALSWKIPVNRRLYHVITTIITLTAALSYFAMATGHGVALNKIVIRTQHDH |
| (LmR) | VPDTYETVYRQVYYARYIDWAITTPLLLLDLGLLAGMSGAHIFMAIVADLIMVLTGLFAA |
| | FGSEGTPQKWGWYTIACIAYIFVVWHLVLNGGANARVKGEKLRSFFVAIGAYTLILWTAY |
| | PIVWGLADGARKIGVDGEIIAYAVLDVLAKGVFGAWLLVTHANLRESD |
| | (SEQ ID NO: 4) |
| Neurospora | MIHPEQVADMLRPTTSTTSSHVPGPVPTVVPTPTEYQTLGETGHRTLWVTFALMVLSSGI |
| crassa | FALLSWNVPTSKRLFHVITTLITVVASLSYFAMATGHATTENCDTAWDHHKHVPDTSHQV |
| (NcR) | CRQVFWGRYVDWALTTPLLLLELCLLAGVDGAHTLMAIVADVIMVLCGLFAALGEGGNTA |
| | QKWGWYTIGCFSYLFVIWHVALHGSRTVTAKGRGVSRLFTGLAVFALLLWTAYPIIWGIA |
| | GGARRTNVDTEILIYTVLDLLAKPVFGFWLLLSHRAMPETN |
| | (SEQ ID NO: 5) |
| Acetabularia | MSNPNPFQTTLGTDAQWVVFAVMALAAIVFSIAVQFRPLPLRLTYYVNIAICTIAATAYY |
| acetabulum | AMAVNGGDNKPTAGTGADERQVIYARYIDWVFTTPLLLLDLVLLTNMPATMIAWIMGADI |
| (Ace1) | AMIAFGIIGAFTVGSYKWFYFVVGCIMLAVLAWGMINPIFKEELQKHKEYTGAYTTLLIY |
| | LIVLWVIYPIVWGLGAGGHIIGVDVEIIAMGVLDLLAKPLYAIGVLITVEVVYGK |
| | (SEQ ID NO: 6) |
| Chlorella | MAVHQIGEGGLVMYWVTFGLMAFSALAFAVMTFTRPLNKRSHGYITLAIVTIAAIAYYAM |
| vulgaris | AASGGKALVSNPDGNLRDIYYARYIDWFFTTPLLLLDIILLTGIPIGVTLWIVLADVAMI |
| (CvR) | MLGLFGALSTNSYRWGYYGVSCAFFFVVLWGLFFPGAKGARARGGQVPGLYFGLAGYLAL |
| | LWFGYPIVWGLAEGSDYISVTAEAASYAGLDIAAKVVFGWAVMLSHPLIARNQ |
| | (SEQ ID NO: 7) |
| Taphrina | MSSLFEKRNTAVATNYRGPTNSIVINEAGSDWYWAVFSVMAASAIVFSVMAAMTPRGERV |
| deformans | FHYLTIAIVSVASVAYFTMAADLGSVAIISEFANYSSLPTRQVFYARYIDWVITTPLLLT |
| (TdR) | DLMLLAGLPWSTIIFTIVMDEVMVLTGLFGAITPSSYKWGYFTFGMVAYFFVAWVLIVEA |
| | RKNAHRLGSDVHRLYIGIAIWTATLWTLYPVAWGLSEGGNVTSSDGEAIFYGVLDLLAKP |
| | VFGLWILLGHKGIGMDRL |
| | (SEQ ID NO: 8) |
| Exophiala | MESNLVRRNGALTVNTMTQNNQSVAIHQTVRGSDWYYTVCAVMGTTSLAILALSRMKPRT |
| sideris | DRVFFYLTSGLCMVACIAYFAMGSNLGWTPIDVEWLRNDSVVRGVNRQVFYARYIDWVIT |
| (EsR) | TPMLLMDLLLTAGMPWPTILWIILLDEIMIVTGLIGALVKSRYKWGFYVFGCMAMFYIMW |
| | ELAFPARKHAKVLGKDIHRSFVLCGVLTLVVWLCYPICWGLSEGGNVISPDSESVFYGVL |
| | DVLAKPSFSIALIATHWNIDPGR |
| | (SEQ ID NO: 9) |
| Moelleriella | MGNSAIEVNGDTVDSYTADVKITTHGSDAYWAITAVMAFTTILFIAHSFTKPRTDRIFHY |
| libera | ITISITLVASIAYFTMASNLGWASIFIEFQRDDPLVSGTTREIFYVRYIDWVITTPLLLL |
| (MIR) | DILLTAGLPWPTILFTILLDEVMIITGLVGALVKSSYKWGFFTFGCVAFFGVAWSVAWTG |
| | RKHANALGSDIGRVYLMTSVWTLFLWLLYPIAWGVSEGGNVISPDSEAAFYGTLDVLAKP |
| | IFGIILLWGHRNIPASR |
| | (SEQ ID NO: 10) |
| Saitoella | MDSLLLQRRNDAVATNPPNATFALTENGSSWLWAVFCVMALSCIIIAALSLFKPMGYRIF |
| complicate | YLLNVAILATASVSYFSLASDLGLTPVTVEFRGPGTRQIAYVRYIDWFVTTPLLLTELLL |
| (ScR) | TAGLPTNIIISTIFADLVMIITGLAGALVVSRYKWGYYTMGCVAMLWVFWNVFTGIKVSG |
| | NIGPDVRKSYTLLAVWLMIIWLNYPICWGLAEGGNRITVVGEMVYYGVLDLLAKPVFAAI |
| | SLAVHSKIELSR |
| | (SEQ ID NO: 11) |
| Rhodotorula | MDAILSKRNEVLSLNPLVANIDITTAASDWLWAVFAVMGLSAIILLVLGHATRPIGERAF |
| graminis | HELAAALCFTASIAYYSMASDLGATPIEVEFIRGGTLGQNWVDIGVLRPTRSIWYARYID |
| (RgR) | WTITTPLLLLELALTTALPLSQIFGLVFFDIVMIITGLLGALTASRYKWGFFVFGCVAMF |
| | WIFWVLFFPARKSASHLGTDYHRAYTSSAIVLCTLWTVYPIIWGVCDGGNVITPTSEMVA |
| | YGVLDLLAKPVFSFWHVFQLSRLDYAR |
| | (SEQ ID NO: 12) |
| Rhodotorula | MPFYIKNADIDITTHGSDWLWAVESVMLLSAIGILVWGHVARPLGERAFHELAAALCFTA |
| toruloides | SIAYFAMASDLGDVPIVVEFIRGGSLGQNWVQVGVENPTRAIWYARYIDWTITTPMLLLE |
| (RtR) | LLLCTGLPLSQVFSVIFADLLMIETGLIGALVASRYKWGFYAFGCAAQLYIWWMLLVPGR |
| | RSAQHIGSDFAKSYTMSNIFLTTVWLVYPVIWGVADGGNVITPDSEMIAYGVLDLLAKPV |
| | FSVIHLMSLSKLDYAR |
| | (SEQ ID NO: 13) |
| Krokinobacter | MKFLLLLLADPTKLDPSDYVGFTFFVGAMAMMAASAFFFLSLNQFNKKWRTSVLVSGLIT |
| eikastus | FIAAVHYWYMRDYWFAIQESPTFFRYVDWVLTVPLMCVEFYLILKVAGAKPALMWKLILF |
| (KeR1) | SVIMLVTGYFGEAVFQDQAALWGAISGAAYFYIVYEIWLGSAKKLAVAAGGDILKAHKIL |
| | CWFVLVGWAIYPLGYMLGTDGWYTSILGKGSVDVAYNIADAINKIGFGLVIYALAVKKNEVD |
| | (SEQ ID NO: 14) |
| Krokinobacter | MTQELGNANFENFIGATEGFSEIAYQFTSHILTLGYAVMLAGLLYFILTIKNVDKKFQMS |
| eikastus | NILSAVVMVSAFLLLYAQAQNWTSSFTFNEEVGRYFLDPSGDLFNNGYRYLNWLIDVPML |
| (KeR2) | LFQILFVVSLTTSKFSSVRNQFWFSGAMMIITGYIGQFYEVSNLTAFLVWGAISSAFFFH |
| | ILWVMKKVINEGKEGISPAGQKILSNIWILFLISWTLYPGAYLMPYLTGVDGFLYSEDGV |
| | MARQLVYTIADVSSKVIYGVLLGNLAITLSKNK |
| | (SEQ ID NO: 15) |
| Gloeobacter | MLMTVFSSAPELALLGSTFAQVDPSNLSVSDSLTYGQFNLVYNAFSFAIAAMFASALFFF |
| violaceus | SAQALVGQRYRLALLVSAIVVSIAGYHYFRIFNSWDAAYVLENGVYSLTSEKFNDAYRYV |
| (GvR) | DWLLTVPLLLVETVAVLTLPAKEARPLLIKLTVASVLMIATGYPGEISDDITTRIIWGTV |
| | STIPFAYILYVLWVELSRSLVRQPAAVQTLVRNMRWLLLLSWGVYPIAYLLPMLGVSGTS |
| | AAVGVQVGYTIADVLAKPVFGLLVFAIALVKTKAD |
| | (SEQ ID NO: 16) |
| Salinibacter | MLQELPTLTPGQYSLVFNMFSFTVATMTASFVFFVLARNNVAPKYRISMMVSALVVFIAG |
| ruber | YHYFRITSSWEAAYALQNGMYQPTGELFNDAYRYVDWLLTVPLLTVELVLVMGLPKNERG |
| (SrR) | PLAAKLGFLAALMIVLGYPGEVSENAALFGTRGLWGFLSTIPFVWILYILFTQLGDTIQR |
| | QSSRVSTLLGNARLLLLATWGFYPIAYMIPMAFPEAFPSNTPGTIVALQVGYTIADVLAK |
| | AGYGVLIYNIAKAKSEEE |
| | (SEQ ID NO: 17) |
| Gammaproteob | MKLLLILGSVIALPTFAAGGGDLDASDYTGVSFWLVTAALLASTVFFFVERDRVSAKWKT |
| acteria | SLTVSGLVTGIAFWHYMYMRGVWIETGDSPTVFRYIDWLLTVPLLICEFYLILAAATNVA |
| (GpR1) | GSLFKKLLVGSLVMLVFGYMGEAGIMAAWPAFIIGCLAWVYMIYELWAGEGKSACNTASP |
| | AVQSAYNTMMYIIIFGWAIYPVGYFTGYLMGDGGSALNLNLIYNLADFVNKILFGLIIWN |
| | VAVKESSN |
| | (SEQ ID NO: 18) |
| Gammaproteobacteria | MGKLLLILGSAIALPSFAAAGGDLDISDTVGVSFWLVTAGMLAATVFFFVERDQVSAKWK |
| (GpR2) | TSLTVSGLITGIAFWHYLYMRGVWIDTGDTPTVFRYIDWLLTVPLQVVEFYLILAACTSV |
| | AASLFKKLLAGSLVMLGAGFAGEAGLAPVLPAFIIGMAGWLYMIYELYMGEGKAAVSTAS |
| | PAVNSAYNAMMMIIVVGWAIYPAGYAAGYLMGGEGVYASNLNLIYNLADFVNKILFGLII |
| | WNVAVKESSN |
| | (SEQ ID NO: 19) |
| Proteomonas | MTMLEHLEGTMDGWYAENDLGQGAIIAHWVTFFFHMITTFYLGYVSFHSKGPGGKQPYFA |
| sulcata | GYHEENNIGIFVNLFAAISYFGKVVSDTHGHNYQNVGPFIIGLGNYRYADYMLTCPLLVM |
| (PsuCCR) | DLLFQLRAPYKITCAMLIFAVLMIGAVTNFYPGDDMKGPAVAWFCFGCFWYLIAYIFMAH |
| | IVSKQYGRLDYLAHGTKAEGALFSLKLAIITFFAIWVAFPLVWLLSVGTGVLSNEAAEIC |
| | HCICDVVAKSVYGFALANFREQYDREL |
| | (SEQ ID NO: 20) |
| Guillardia theta | MVESSAVIAANWISFLVIAGSFVVLCFISLRYKGPGGNENYYNGFREQNMLTVIINLWCA |
| (GtCCR1) | LAYFAKVLQSHSDDDGFVPLTKIPYLDYATTCPLLTLDLMWCLDAPYKITSAVLVFTVMI |
| | TGVACSLAVAPYSFYWFAMGMVLFIFTYVLMLSIVRERLEFITQCAHDSNAKRSIKHLKA |
| | AVIIYFGIWPIFAILWLLSYRAANVISNDTNHILHCILDVIAKSCFGFVLLHFKMYFDKKL |
| | (SEQ ID NO: 21) |
| Guillardia theta | MVASSAVITANWISFLAISASFIILLVISLRYKGPGGTESFYNGFKEQNMLTVFINLWCA |
| (GtCCR2) | LAYFAKVLQSHSNDNGFAPLTVIPYVDYCTTCPLLTLDLLWCLDAPYKISSAVLVFTCLV |
| | IAVACSLAVAPFSYCWFAMGMVLFTFTYVFILSIVRQRLDFFTLCARDSNAKQSLKHLKT |
| | AVFIYFGIWLLFPLLWLLSYRAANVISNDINHIFHCILDVIAKSVYGFALLYFKMYFDKKL |
| | (SEQ ID NO: 22) |
| Guillardia theta | MVSALDQNGPQYLQNPIVIAADWIGFIALFGSSLAVAYKLVTFKGPDQDDVYFFGYREEK |
| (GtCCR3) | MISVFVNLFAALAYWAKLASHANGDVGPAASVTTYKYLDYLFTCPLLTIDLLWCLNLPYK |
| | FTFGAIVAVCILCAFMASVIPPPARYMWFGMGITVFSAAWFNILKLVRMRLEQFVSKEAK |
| | KVRQSLKVACMTYFFIWLGYPTLWVLGDAGVLDSVVSALLHTFLDVFSKSIYGFALLHFV |
| | MRTDKRE |
| | (SEQ ID NO: 23) |
| Guillardia theta | MTTSAPSLSDPNWQYGMGGWNNPRLPNFNLHDPTVIGVDWLGFLCLLGASLALMYKLMSF |
| (GtCCR4) | KGPDGDQEFFVGYREEKCLSIYVNLIAAITYWGRICAHFNNDMGLSLSVNYFKYLDYIFT |
| | CPILTLDLLWSLNLPYKITYSLFVGLTIACNAFEPPARYLWFMFGCFIFAFTWISIIRLV |
| | YARFQQFLNEDAKKIRAPLKLSLTLYFSIWCGYPALWLLTEFGAISQLAAHVMTVIMDVA |
| | AKSVYGFALLKFQLGVDKRD |
| | (SEQ ID NO: 24) |
| Guillardia theta | MSTSSVAYLRTPVVQALDWVGFISLGGTAAVLAYRLMNFKPPNKDILYFFGYREKGMISL |
| (GtCCR5) | YVNLFAAVAYYARITSHLSGDVGAATNIILYKYFDYLITCPLLTFDLLTTLNLPYKITYA |
| | VYVQITIFTGFMSANTPPPATFLWFAFGMLLFSYTWFNIISLVQVRFIQYFAKKGNTTQS |
| | RRVSVASKAGFRNKNVRNPLQTALSTYFCIWMVYPVLWLLLKTKVIDQVTEHCINVVMDV |
| | LAKSMYGFALLRFQLLMDKAN |
| | (SEQ ID NO: 25) |
| Actinomycetes | MAKPTVKEIKSLQNFNRIAGVFHLLQMLAVLALANDFALPMTGTYLNGPPGTTFSAPVVI |
| bacterium | LETPVGLAVALFLGLSALFHFIVSSGNFFKRYSASLMKNQNIFRWVEYSLSSSVMIVLIA |
| (AbR) | QICGIADIVALLAIFGVNASMILFGWLQEKYTQPKDGDLLPFWFGCIAGIVPWIGLLIYV |
| | IAPGSTSDVAVPGFVYGIIISLFLFFNSFALVQYLQYKGKGKWSNYLRGERAYIVLSLVA |
| | KSALAWQIFSGTLIP |
| | (SEQ ID NO: 26) |
| Emiliania | MDTIVLTINWFNFIAGLFHVALAIVCALLGDVSQTFKMYMPFTTFRNGTQENEVTIKYSG |
| huxleyi | YFPMTVIFVVYFSVTALFHMGNAFLWNDTYHRFLSNRKNPIRWTEYSITAPLMTAILAFI |
| (EhR) | AGSRNWLFVIAASTLTFASISVGFVLDEIRKYSFFVLGMIPFLVEFSLIILSLYLTTSCY |
| | PSYLPITIFVEFGLWILFPFVSLLELSGINYKIGELVFIVLSFVSKSVLAIILNSENVLK |
| | NGVTGIC |
| | (SEQ ID NO: 27) |
| Heimdallarchaeota | MTKALEMTILDDGSESPISFKYLRKFNLAMGILHLVQGVAMLVLGFFWDFSRPLYSITLD |
| archaeon | YSGGPPVAALQPEFSFTAVGPTVAAFLLFSALAHLLIAGPLNKFYVKNLKKKMNPIRWFE |
| (HaR) | YAFSSSIMVFFIAILFGVWDLWVLIGLFFLNMLMNLFGHMMELHNQTTKKTNWTAYIYGW |
| | IAGIIPWVIISVFFARIAINSTGMPWFVPVIYAFELILFMSFAFNMLLQYKKVGKWKDYL |
| | YGERMYQILSLVAKTLLAWLVFAGVFQPA (SEQ ID NO: 28) |
| Halobacterium | MSSHSATTSSGRRESARDSRLRLWNSVMAVLHFLQGAAMVLLADTVLWPITRTRYGFDPG |
| sp.DL1 | SQSIFPETVAFVDANLPLLVAGFLFISALAHTAIATVWYDKYVRYLDRGMNPYRWYEYSV |
| (HdR) | SASLMIVVIGMLSGVWDLGTLVALFGLVAVMNLSGLLMEQRNELTEQTDWTPYWVGVIAG |
| | IVPWITIGVAFVGSVTASAGEFPEFVIYIYVSIFVFFNLFALNMALQYLEVSRWKNYLFG |
| | EKMYIVLSLVAKSALAWQVYFGTLNSPI |
| | (SEQ ID NO: 29) |
| Thermoplasmatales | MTDYEDHTIRRFRIFNAIMGGIHLLQVFLVLYLSNNFSLPVTISKPVYNEFTNSISPVSE |
| archaeon | TLFSIRVGPLVALFLFISAIAHILIATVLYYRYVENLKSGMNPYRWFEYSISASVMIVII |
| SG8 | AMLTTIYDLGTLLALFTLTAVMNLMGLMMELHNQTTQNTDWTSYIIGCIAGLVPWIVIFI |
| (TsR) | PLIAAESVPDFVIYIFVSIAIFFNCFAINMYLQYKKIGKWKDYLHGERVYIILSLVAKSA |
| | LAWQVFAGTLRPM |
| | (SEQ ID NO: 30) |

TABLE 2
Protein sequence of all constructs generated by fusing HaloTag to the c-terminus of
rhodopsins tested and introducing mutations to generate putative negative-going or
positive-going voltage indicators. Mutation: bold underline.

| Construct Name | Sequence |
|---|---|
| KnR (−) | MVYSHADAAGWTLYWITYGIMAVTALIFFAMSLRRPIQQRSHHYTSFLIVAIASLAYYAM |
| | ASQGGNTRIRVYQGPADSYRQIFWARYVNWFFTTPLLLLDLVLLSNLSKLRIAAIMVADI |
| | FMILTGLFGAVEARSNKWGWFVFGCIFMLYIFYELLVNVRKGAYARGGQHGMLYSVLLVW |
| | LLILWVQYPVVWGLAEGSSTVSSDTEIAWYAALDICAKCVFGFILLLGIESIDRKRIGTGFP |
| | FDPHYVEVLGERMHYVDVGPRDGTPVLFLHGNPTSSYVWRNIIPHVAPTHRCIAPDLI |
| | GMGKSDKPDLGYFFDDHVRFMDAFIEALGLEEVVLVIHDWGSALGFHWAKRNPERVKGIA |
| | FMEFIRPIPTWDEWPEFARETFQAFRTTDVGRKLIIDQNVFIEGTLPMGVVRPLTEVEMD |
| | HYREPFLNPVDREPLWRFPNELPIAGEPANIVALVEEYMDWLHQSPVPKLLFWGTPGVLI |
| | PPAEAARLAKSLPNCKAVDIGPGLNLLQEDNPDLIGSEIARWLSTLEISGEPTTKSRITSEG |
| | EYIPLDQIDINVFCYENEV |
| | (SEQ ID NO: 83) |
| PaR (−) | MIHHDQVAEMLYGYAGAAAAKPTHTDSPGPIPTVIPTPPQFQEIGETGHRTLWVVFALMV |
| | LSSGFFAFMSWNVPISKRLYHVITTLITITASLSYFAMASGHVTSFSCTPAKDHHKHVPD |
| | VGYTECRQVFWGRYVNWAITTPLLLLDLSLLAGIDGAHTLMAVIADVIMVLSGLFASQGE |
| | TATQRWGWYAIGCVSYLFVIWHVALHGARTVTAKGRGVTRLFSSLALFTFVLWTAYPIVW |
| | GIADGAHRTTVDTEILIYAVLDILAKPVFGLWLLFSHRSLAETNIGTGFPFDPHYVEVLG |
| | ERMHYVDVGPRDGTPVLFLHGNPTSSYVWRNIIPHVAPTHRCIAPDLIGMGKSDKPDLGY |
| | FFDDHVRFMDAFIEALGLEEVVLVIHDWGSALGFHWAKRNPERVKGIAFMEFIRPIPTWD |
| | EWPEFARETFQAFRTTDVGRKLIIDQNVFIEGTLPMGVVRPLTEVEMDHYREPFLNPVDR |
| | EPLWRFPNELPIAGEPANIVALVEEYMDWLHQSPVPKLLFWGTPGVLIPPAEAARLAKSL |
| | PNCKAVDIGPGLNLLQEDNPDLIGSEIARWLSTLEISGEPTTKSRITSEGEYIPLDQIDINV |
| | FCYENEV |
| | (SEQ ID NO: 88) |
| CR1 (−) | MPEPGSEAIWLWLGTAGMFLGMLYFIARGWGETDSRRQKFYIATILITAIAFVNYLAMAL |
| | GFGLTIVEFAGEEHPIYWARYSNWLFTTPLLLYDLGLLAGADRNTITSLVSLDVLMIGTG |
| | LVATLSPGSGVLSAGAERLVWWGISTAFLLVLLYFLFSSLSGRVADLPSDTRSTFKTLRN |
| | LVTVVWLVYPVWWLIGTEGIGLVGIGIETAGFMVIDLTAKVGFGIILLRSHGVLDGAAIG |
| | TGFPFDPHYVEVLGERMHYVDVGPRDGTPVLFLHGNPTSSYVWRNIIPHVAPTHRCIAPD |
| | LIGMGKSDKPDLGYFFDDHVRFMDAFIEALGLEEVVLVIHDWGSALGFHWAKRNPERVKG |
| | IAFMEFIRPIPTWDEWPEFARETFQAFRTTDVGRKLIIDQNVFIEGTLPMGVVRPLTEVE |
| | MDHYREPFLNPVDREPLWRFPNELPIAGEPANIVALVEEYMDWLHQSPVPKLLFWGTPGV |
| | LIPPAEAARLAKSLPNCKAVDIGPGLNLLQEDNPDLIGSEIARWLSTLEISGEPTTKSRI |
| | TSEGEYIPLDQIDINVFCYENEV |
| | (SEQ ID NO: 91) |
| LmR (−) | MIVDQFEEVLMKTSQLFPLPTATQSAQPTHVAPVPTVLPDTPIYETVGDSGSKTLWVVFV |
| | LMLIASAAFTALSWKIPVNRRLYHVITTIITLTAALSYFAMATGHGVALNKIVIRTQHDH |
| | VPDTYETVYRQVYYARYINWAITTPLLLLDLGLLAGMSGAHIFMAIVADLIMVLTGLFAA |
| | FGSEGTPQKWGWYTIACIAYIFVVWHLVLNGGANARVKGEKLRSFFVAIGAYTLILWTAY |
| | PIVWGLADGARKIGVDGEIIAYAVLDVLAKGVFGAWLLVTHANLRESDIGTGFPFDPHYV |
| | EVLGERMHYVDVGPRDGTPVLFLHGNPTSSYVWRNIIPHVAPTHRCIAPDLIGMGKSDKP |
| | DLGYFFDDHVRFMDAFIEALGLEEVVLVIHDWGSALGFHWAKRNPERVKGIAFMEFIRPI |
| | PTWDEWPEFARETFQAFRTTDVGRKLIIDQNVFIEGTLPMGVVRPLTEVEMDHYREPFLN |
| | PVDREPLWRFPNELPIAGEPANIVALVEEYMDWLHQSPVPKLLFWGTPGVLIPPAEAARL |
| | AKSLPNCKAVDIGPGLNLLQEDNPDLIGSEIARWLSTLEISGEPTTKSRITSEGEYIPLD |
| | QIDINVFCYENEV |
| | (SEQ ID NO: 92) |
| NcR (−) | MIHPEQVADMLRPTTSTTSSHVPGPVPTVVPTPTEYQTLGETGHRTLWVTFALMVLSSGI |
| | FALLSWNVPTSKRLFHVITTLITVVASLSYFAMATGHATTENCDTAWDHHKHVPDTSHQV |
| | CRQVFWGRYVNWALTTPLLLLELCLLAGVDGAHTLMAIVADVIMVLCGLFAALGEGGNTA |
| | QKWGWYTIGCFSYLFVIWHVALHGSRTVTAKGRGVSRLFTGLAVFALLLWTAYPIIWGIA |
| | GGARRTNVDTEILIYTVLDLLAKPVFGFWLLLSHRAMPETNIGTGFPFDPHYVEVLGERM |
| | HYVDVGPRDGTPVLFLHGNPTSSYVWRNIIPHVAPTHRCIAPDLIGMGKSDKPDLGYFFD |
| | DHVRFMDAFIEALGLEEVVLVIHDWGSALGFHWAKRNPERVKGIAFMEFIRPIPTWDEWP |
| | EFARETFQAFRTTDVGRKLIIDQNVFIEGTLPMGVVRPLTEVEMDHYREPFLNPVDREPL |
| | WRFPNELPIAGEPANIVALVEEYMDWLHQSPVPKLLFWGTPGVLIPPAEAARLAKSLPNC |
| | KAVDIGPGLNLLQEDNPDLIGSEIARWLSTLEISGEPTTKSRITSEGEYIPLDQIDINVF |
| | CYENEV |
| | (SEQ ID NO: 93) |
| Ace1 (−) | MSNPNPFQTTLGTDAQWVVFAVMALAAIVFSIAVQFRPLPLRLTYYVNIAICTIAATAYY |
| | AMAVNGGDNKPTAGTGADERQVIYARYINWVFTTPLLLLDLVLLTNMPATMIAWIMGADI |
| | AMIAFGIIGAFTVGSYKWFYFVVGCIMLAVLAWGMINPIFKEELQKHKEYTGAYTTLLIY |
| | LIVLWVIYPIVWGLGAGGHIIGVDVEIIAMGVLDLLAKPLYAIGVLITVEVVYGKIGTGF |
| | PFDPHYVEVLGERMHYVDVGPRDGTPVLFLHGNPTSSYVWRNIIPHVAPTHRCIAPDLIG |
| | MGKSDKPDLGYFFDDHVRFMDAFIEALGLEEVVLVIHDWGSALGFHWAKRNPERVKGIAF |
| | MEFIRPIPTWDEWPEFARETFQAFRTTDVGRKLIIDQNVFIEGTLPMGVVRPLTEVEMDH |
| | YREPFLNPVDREPLWRFPNELPIAGEPANIVALVEEYMDWLHQSPVPKLLFWGTPGVLIP |
| | PAEAARLAKSLPNCKAVDIGPGLNLLQEDNPDLIGSEIARWLSTLEISGEPTTKSRITSE |
| | GEYIPLDQIDINVFCYENEV |
| | (SEQ ID NO: 94) |
| CvR (−) | MAVHQIGEGGLVMYWVTFGLMAFSALAFAVMTFTRPLNKRSHGYITLAIVTIAAIAYYAM |
| | AASGGKALVSNPDGNLRDIYYARYINWFFTTPLLLLDIILLTGIPIGVTLWIVLADVAMI |
| | MLGLFGALSTNSYRWGYYGVSCAFFFVVLWGLFFPGAKGARARGGQVPGLYFGLAGYLAL |
| | LWFGYPIVWGLAEGSDYISVTAEAASYAGLDIAAKVVFGWAVMLSHPLIARNQIGTGFPF |
| | DPHYVEVLGERMHYVDVGPRDGTPVLFLHGNPTSSYVWRNIIPHVAPTHRCIAPDLIGMG |
| | KSDKPDLGYFFDDHVRFMDAFIEALGLEEVVLVIHDWGSALGFHWAKRNPERVKGIAFME |
| | FIRPIPTWDEWPEFARETFQAFRTTDVGRKLIIDQNVFIEGTLPMGVVRPLTEVEMDHYR |
| | EPFLNPVDREPLWRFPNELPIAGEPANIVALVEEYMDWLHQSPVPKLLFWGTPGVLIPPA |
| | EAARLAKSLPNCKAVDIGPGLNLLQEDNPDLIGSEIARWLSTLEISGEPTTKSRITSEGE |
| | YIPLDQIDINVFCYENEV |
| | (SEQ ID NO: 95) |
| TdR (−) | MSSLFEKRNTAVATNYRGPTNSIVINEAGSDWYWAVFSVMAASAIVFSVMAAMTPRGERV |
| | FHYLTIAIVSVASVAYFTMAADLGSVAIISEFANYSSLPTRQVFYARYINWVITTPLLLT |
| | DLMLLAGLPWSTIIFTIVMDEVMVLTGLFGAITPSSYKWGYFTFGMVAYFFVAWVLIVEA |
| | RKNAHRLGSDVHRLYIGIAIWTATLWTLYPVAWGLSEGGNVTSSDGEAIFYGVLDLLAKP |
| | VFGLWILLGHKGIGMDRIGTGFPFDPHYVEVLGERMHYVDVGPRDGTPVLFLHGNPTSSY |
| | VWRNIIPHVAPTHRCIAPDLIGMGKSDKPDLGYFFDDHVRFMDAFIEALGLEEVVLVIHD |
| | WGSALGFHWAKRNPERVKGIAFMEFIRPIPTWDEWPEFARETFQAFRTTDVGRKLIIDQN |
| | VFIEGTLPMGVVRPLTEVEMDHYREPFLNPVDREPLWRFPNELPIAGEPANIVALVEEYM |
| | DWLHQSPVPKLLFWGTPGVLIPPAEAARLAKSLPNCKAVDIGPGLNLLQEDNPDLIGSEI |
| | ARWLSTLEISGEPTTKSRITSEGEYIPLDQIDINVFCYENEV |
| | (SEQ ID NO: 96) |
| EsR (−) | MESNLVRRNGALTVNTMTQNNQSVAIHQTVRGSDWYYTVCAVMGTTSLAILALSRMKPRT |
| | DRVFFYLTSGLCMVACIAYFAMGSNLGWTPIDVEWLRNDSVVRGVNRQVFYARYINWVIT |
| | TPMLLMNLLLTAGMPWPTILWIILLDEIMIVTGLIGALVKSRYKWGFYVFGCMAMFYIMW |
| | ELAFPARKHAKVLGKDIHRSFVLCGVLTLVVWLCYPICWGLSEGGNVISPDSESVFYGVL |
| | DVLAKPSFSIALIATHWNIDPGRIGTGFPFDPHYVEVLGERMHYVDVGPRDGTPVLFLHG |
| | NPTSSYVWRNIIPHVAPTHRCIAPDLIGMGKSDKPDLGYFFDDHVRFMDAFIEALGLEEV |
| | VLVIHDWGSALGFHWAKRNPERVKGIAFMEFIRPIPTWDEWPEFARETFQAFRTTDVGRK |
| | LIIDQNVFIEGTLPMGVVRPLTEVEMDHYREPFLNPVDREPLWRFPNELPIAGEPANIVA |
| | LVEEYMDWLHQSPVPKLLFWGTPGVLIPPAEAARLAKSLPNCKAVDIGPGLNLLQEDNPD |
| | LIGSEIARWLSTLEISGEPTTKSRITSEGEYIPLDQIDINVFCYENEV |
| | (SEQ ID NO: 97) |
| MIR (−) | MGNSAIEVNGDTVDSYTADVKITTHGSDAYWAITAVMAFTTILFIAHSFTKPRTDRIFHY |
| | ITISITLVASIAYFTMASNLGWASIFIEFQRDDPLVSGTTREIFYVRYINWVITTPLLLL |
| | NILLTAGLPWPTILFTILLDEVMIITGLVGALVKSSYKWGFFTFGCVAFFGVAWSVAWTG |
| | RKHANALGSDIGRVYLMTSVWTLFLWLLYPIAWGVSEGGNVISPDSEAAFYGTLDVLAKP |
| | IFGIILLWGHRNIPASRIGTGFPFDPHYVEVLGERMHYVDVGPRDGTPVLFLHGNPTSSY |
| | VWRNIIPHVAPTHRCIAPDLIGMGKSDKPDLGYFFDDHVRFMDAFIEALGLEEVVLVIHD |
| | WGSALGFHWAKRNPERVKGIAFMEFIRPIPTWDEWPEFARETFQAFRTTDVGRKLIIDQN |
| | VFIEGTLPMGVVRPLTEVEMDHYREPFLNPVDREPLWRFPNELPIAGEPANIVALVEEYM |
| | DWLHQSPVPKLLFWGTPGVLIPPAEAARLAKSLPNCKAVDIGPGLNLLQEDNPDLIGSEI |
| | ARWLSTLEISGEPTTKSRITSEGEYIPLDQIDINVFCYENEV |
| | (SEQ ID NO: 98) |
| ScR (−) | MDSLLLQRRNDAVATNPPNATFALTENGSSWLWAVFCVMALSCIIIAALSLFKPMGYRIF |
| | YLLNVAILATASVSYFSLASDLGLTPVTVEFRGPGTRQIAYVRYINWFVTTPLLLTELLL |
| | TAGLPTNIIISTIFADLVMIITGLAGALVVSRYKWGYYTMGCVAMLWVFWNVFTGIKVSG |
| | NIGPDVRKSYTLLAVWLMIIWLNYPICWGLAEGGNRITVVGEMVYYGVLDLLAKPVFAAI |
| | SLAVHSKIELSRIGTGFPFDPHYVEVLGERMHYVDVGPRDGTPVLFLHGNPTSSYVWRNI |
| | IPHVAPTHRCIAPDLIGMGKSDKPDLGYFFDDHVRFMDAFIEALGLEEVVLVIHDWGSAL |
| | GFHWAKRNPERVKGIAFMEFIRPIPTWDEWPEFARETFQAFRTTDVGRKLIIDQNVFIEG |
| | TLPMGVVRPLTEVEMDHYREPFLNPVDREPLWRFPNELPIAGEPANIVALVEEYMDWLHQ |
| | SPVPKLLFWGTPGVLIPPAEAARLAKSLPNCKAVDIGPGLNLLQEDNPDLIGSEIARWLS |
| | TLEISGEPTTKSRITSEGEYIPLDQIDINVFCYENEV |
| | (SEQ ID NO: 99) |
| RgR (−) | MDAILSKRNEVLSLNPLVANIDITTAASDWLWAVFAVMGLSAIILLVLGHATRPIGERAF |
| | HELAAALCFTASIAYYSMASDLGATPIEVEFIRGGTLGQNWVDIGVLRPTRSIWYARYIN |
| | WTITTPLLLLELALTTALPLSQIFGLVFFDIVMIITGLLGALTASRYKWGFFVFGCVAMF |
| | WIFWVLFFPARKSASHLGTDYHRAYTSSAIVLCTLWTVYPIIWGVCDGGNVITPTSEMVA |
| | YGVLDLLAKPVFSFWHVFQLSRLDYARIGTGFPFDPHYVEVLGERMHYVDVGPRDGTPVL |
| | FLHGNPTSSYVWRNIIPHVAPTHRCIAPDLIGMGKSDKPDLGYFFDDHVRFMDAFIEALG |
| | LEEVVLVIHDWGSALGFHWAKRNPERVKGIAFMEFIRPIPTWDEWPEFARETFQAFRTTD |
| | VGRKLIIDQNVFIEGTLPMGVVRPLTEVEMDHYREPFLNPVDREPLWRFPNELPIAGEPA |
| | NIVALVEEYMDWLHQSPVPKLLFWGTPGVLIPPAEAARLAKSLPNCKAVDIGPGLNLLQE |
| | DNPDLIGSEIARWLSTLEISGEPTTKSRITSEGEYIPLDQIDINVFCYENEV |
| | (SEQ ID NO: 100) |
| RtR (−) | MPFYIKNADIDITTHGSDWLWAVFSVMLLSAIGILVWGHVARPLGERAFHELAAALCFTA |
| | SIAYFAMASDLGDVPIVVEFIRGGSLGQNWVQVGVENPTRAIWYARYINWTITTPMLLLE |
| | LLLCTGLPLSQVFSVIFADLLMIETGLIGALVASRYKWGFYAFGCAAQLYIWWMLLVPGR |
| | RSAQHIGSDFAKSYTMSNIFLTTVWLVYPVIWGVADGGNVITPDSEMIAYGVLDLLAKPV |
| | FSVIHLMSLSKLDYARIGTGFPFDPHYVEVLGERMHYVDVGPRDGTPVLFLHGNPTSSYV |
| | WRNIIPHVAPTHRCIAPDLIGMGKSDKPDLGYFFDDHVRFMDAFIEALGLEEVVLVIHDW |
| | GSALGFHWAKRNPERVKGIAFMEFIRPIPTWDEWPEFARETFQAFRTTDVGRKLIIDQNV |
| | FIEGTLPMGVVRPLTEVEMDHYREPFLNPVDREPLWRFPNELPIAGEPANIVALVEEYMD |
| | WLHQSPVPKLLFWGTPGVLIPPAEAARLAKSLPNCKAVDIGPGLNLLQEDNPDLIGSEIA |
| | RWLSTLEISGEPTTKSRITSEGEYIPLDQIDINVFCYENEV |
| | (SEQ ID NO: 101) |
| GpR1 (−) | MKLLLILGSVIALPTFAAGGGDLDASDYTGVSFWLVTAALLASTVFFFVERDRVSAKWKT |
| | SLTVSGLVTGIAFWHYMYMRGVWIETGDSPTVFRYINWLLTVPLLICEFYLILAAATNVA |
| | GSLFKKLLVGSLVMLVFGYMGEAGIMAAWPAFIIGCLAWVYMIYELWAGEGKSACNTASP |
| | AVQSAYNTMMYIIIFGWAIYPVGYFTGYLMGDGGSALNLNLIYNLADFVNKILFGLIIWN |
| | VAVKESSNIGTGFPFDPHYVEVLGERMHYVDVGPRDGTPVLFLHGNPTSSYVWRNIIPHV |
| | APTHRCIAPDLIGMGKSDKPDLGYFFDDHVRFMDAFIEALGLEEVVLVIHDWGSALGFHW |
| | AKRNPERVKGIAFMEFIRPIPTWDEWPEFARETFQAFRTTDVGRKLIIDQNVFIEGTLPM |
| | GVVRPLTEVEMDHYREPFLNPVDREPLWRFPNELPIAGEPANIVALVEEYMDWLHQSPVP |
| | KLLFWGTPGVLIPPAEAARLAKSLPNCKAVDIGPGLNLLQEDNPDLIGSEIARWLSTLEI |
| | SGEPTTKSRITSEGEYIPLDQIDINVFCYENEV |
| | (SEQ ID NO: 102) |
| GpR2 (−) | MGKLLLILGSAIALPSFAAAGGDLDISDTVGVSFWLVTAGMLAATVFFFVERDQVSAKWK |
| | TSLTVSGLITGIAFWHYLYMRGVWIDTGDTPTVFRYINWLLTVPLQVVEFYLILAACTSV |
| | AASLFKKLLAGSLVMLGAGFAGEAGLAPVLPAFIIGMAGWLYMIYELYMGEGKAAVSTAS |
| | PAVNSAYNAMMMIIVVGWAIYPAGYAAGYLMGGEGVYASNLNLIYNLADFVNKILFGLII |
| | WNVAVKESSNIGTGFPFDPHYVEVLGERMHYVDVGPRDGTPVLFLHGNPTSSYVWRNIIP |
| | HVAPTHRCIAPDLIGMGKSDKPDLGYFFDDHVRFMDAFIEALGLEEVVLVIHDWGSALGF |
| | HWAKRNPERVKGIAFMEFIRPIPTWDEWPEFARETFQAFRTTDVGRKLIIDQNVFIEGTL |
| | PMGVVRPLTEVEMDHYREPFLNPVDREPLWRFPNELPIAGEPANIVALVEEYMDWLHQSP |
| | VPKLLFWGTPGVLIPPAEAARLAKSLPNCKAVDIGPGLNLLQEDNPDLIGSEIARWLSTL |
| | EISGEPTTKSRITSEGEYIPLDQIDINVFCYENEV |
| | (SEQ ID NO: 103) |
| KeR1 (−) | MKFLLLLLADPTKLDPSDYVGFTFFVGAMAMMAASAFFFLSLNQFNKKWRTSVLVSGLIT |
| | FIAAVHYWYMRDYWFAIQESPTFFRYVNWVLTVPLMCVEFYLILKVAGAKPALMWKLILF |
| | SVIMLVTGYFGEAVFQDQAALWGAISGAAYFYIVYEIWLGSAKKLAVAAGGDILKAHKIL |
| | CWFVLVGWAIYPLGYMLGTDGWYTSILGKGSVDVAYNIADAINKIGFGLVIYALAVKKNE |
| | VDIGTGFPFDPHYVEVLGERMHYVDVGPRDGTPVLFLHGNPTSSYVWRNIIPHVAPTHRC |
| | IAPDLIGMGKSDKPDLGYFFDDHVRFMDAFIEALGLEEVVLVIHDWGSALGFHWAKRNPE |
| | RVKGIAFMEFIRPIPTWDEWPEFARETFQAFRTTDVGRKLIIDQNVFIEGTLPMGVVRPL |
| | TEVEMDHYREPFLNPVDREPLWRFPNELPIAGEPANIVALVEEYMDWLHQSPVPKLLFWG |
| | TPGVLIPPAEAARLAKSLPNCKAVDIGPGLNLLQEDNPDLIGSEIARWLSTLEISGEPTT |
| | KSRITSEGEYIPLDQIDINVFCYENEV |
| | (SEQ ID NO: 104) |
| KeR2 (−) | MTQELGNANFENFIGATEGFSEIAYQFTSHILTLGYAVMLAGLLYFILTIKNVDKKFQMS |
| | NILSAVVMVSAFLLLYAQAQNWTSSFTFNEEVGRYFLDPSGDLFNNGYRYLNWLINVPML |
| | LFQILFVVSLTTSKFSSVRNQFWFSGAMMIITGYIGQFYEVSNLTAFLVWGAISSAFFFH |
| | ILWVMKKVINEGKEGISPAGQKILSNIWILFLISWTLYPGAYLMPYLTGVDGFLYSEDGV |
| | MARQLVYTIADVSSKVIYGVLLGNLAITLSKNKIGTGFPFDPHYVEVLGERMHYVDVGPR |
| | DGTPVLFLHGNPTSSYVWRNIIPHVAPTHRCIAPDLIGMGKSDKPDLGYFFDDHVRFMDA |
| | FIEALGLEEVVLVIHDWGSALGFHWAKRNPERVKGIAFMEFIRPIPTWDEWPEFARETFQ |
| | AFRTTDVGRKLIIDQNVFIEGTLPMGVVRPLTEVEMDHYREPFLNPVDREPLWRFPNELP |
| | IAGEPANIVALVEEYMDWLHQSPVPKLLFWGTPGVLIPPAEAARLAKSLPNCKAVDIGPG |
| | LNLLQEDNPDLIGSEIARWLSTLEISGEPTTKSRITSEGEYIPLDQIDINVFCYENEV |
| | (SEQ ID NO: 105) |
| GvR (−) | MLMTVFSSAPELALLGSTFAQVDPSNLSVSDSLTYGQFNLVYNAFSFAIAAMFASALFFF |
| | SAQALVGQRYRLALLVSAIVVSIAGYHYFRIFNSWDAAYVLENGVYSLTSEKFNDAYRYV |
| | NWLLTVPLLLVETVAVLTLPAKEARPLLIKLTVASVLMIATGYPGEISDDITTRIIWGTV |
| | STIPFAYILYVLWVELSRSLVRQPAAVQTLVRNMRWLLLLSWGVYPIAYLLPMLGVSGTS |
| | AAVGVQVGYTIADVLAKPVFGLLVFAIALVKTKADIGTGFPFDPHYVEVLGERMHYVDVG |
| | PRDGTPVLFLHGNPTSSYVWRNIIPHVAPTHRCIAPDLIGMGKSDKPDLGYFFDDHVRFM |
| | DAFIEALGLEEVVLVIHDWGSALGFHWAKRNPERVKGIAFMEFIRPIPTWDEWPEFARET |
| | FQAFRTTDVGRKLIIDQNVFIEGTLPMGVVRPLTEVEMDHYREPFLNPVDREPLWRFPNE |
| | LPIAGEPANIVALVEEYMDWLHQSPVPKLLFWGTPGVLIPPAEAARLAKSLPNCKAVDIG |
| | PGLNLLQEDNPDLIGSEIARWLSTLEISGEPTTKSRITSEGEYIPLDQIDINVFCYENEV |
| | (SEQ ID NO: 106) |
| SrR (−) | MLQELPTLTPGQYSLVFNMFSFTVATMTASFVFFVLARNNVAPKYRISMMVSALVVFIAG |
| | YHYFRITSSWEAAYALQNGMYQPTGELFNDAYRYVNWLLTVPLLTVELVLVMGLPKNERG |
| | PLAAKLGFLAALMIVLGYPGEVSENAALFGTRGLWGFLSTIPFVWILYILFTQLGDTIQR |
| | QSSRVSTLLGNARLLLLATWGFYPIAYMIPMAFPEAFPSNTPGTIVALQVGYTIADVLAK |
| | AGYGVLIYNIAKAKSEEEIGTGFPFDPHYVEVLGERMHYVDVGPRDGTPVLFLHGNPTSS |
| | YVWRNIIPHVAPTHRCIAPDLIGMGKSDKPDLGYFFDDHVRFMDAFIEALGLEEVVLVIH |
| | DWGSALGFHWAKRNPERVKGIAFMEFIRPIPTWDEWPEFARETFQAFRTTDVGRKLIIDQ |
| | NVFIEGTLPMGVVRPLTEVEMDHYREPFLNPVDREPLWRFPNELPIAGEPANIVALVEEY |
| | MDWLHQSPVPKLLFWGTPGVLIPPAEAARLAKSLPNCKAVDIGPGLNLLQEDNPDLIGSE |
| | IARWLSTLEISGEPTTKSRITSEGEYIPLDQIDINVFCYENEV |
| | (SEQ ID NO: 107) |
| AbR (−) | MAKPTVKEIKSLQNFNRIAGVFHLLQMLAVLALANDFALPMTGTYLNGPPGTTFSAPVVI |
| | LETPVGLAVALFLGLSALFHFIVSSGNFFKRYSASLMKNQNIFRWVQYSLSSSVMIVLIA |
| | QICGIADIVALLAIFGVNASMILFGWLQEKYTQPKDGDLLPFWFGCIAGIVPWIGLLIYV |
| | IAPGSTSDVAVPGFVYGIIISLFLFFNSFALVQYLQYKGKGKWSNYLRGERAYIVLSLVA |
| | KSALAWQIFSGTLIPAIGTGFPFDPHYVEVLGERMHYVDVGPRDGTPVLFLHGNPTSSYV |
| | WRNIIPHVAPTHRCIAPDLIGMGKSDKPDLGYFFDDHVRFMDAFIEALGLEEVVLVIHDW |
| | GSALGFHWAKRNPERVKGIAFMEFIRPIPTWDEWPEFARETFQAFRTTDVGRKLIIDQNV |
| | FIEGTLPMGVVRPLTEVEMDHYREPFLNPVDREPLWRFPNELPIAGEPANIVALVEEYMD |
| | WLHQSPVPKLLFWGTPGVLIPPAEAARLAKSLPNCKAVDIGPGLNLLQEDNPDLIGSEIA |
| | RWLSTLEISGEPTTKSRITSEGEYIPLDQIDINVFCYENEV |
| | (SEQ ID NO: 108) |
| HdR (−) | MSSHSATTSSGRRESARDSRLRLWNSVMAVLHFLQGAAMVLLADTVLWPITRTRYGFDPG |
| | SQSIFPETVAFVDANLPLLVAGFLFISALAHTAIATVWYDKYVRYLDRGMNPYRWYEYSV |
| | SASLMIVVIGMLSGVWDLGTLVALFGLVAVMNLSGLLMEQRNELTEQTDWTPYWVGVIAG |
| | IVPWITIGVAFVGSVTASAGEFPEFVIYIYVSIFVFFNLFALNMALQYLEVSRWKNYLFG |
| | EKMYIVLSLVAKSALAWQVYFGTLNSPIIGTGFPFDPHYVEVLGERMHYVDVGPRDGTPV |
| | LFLHGNPTSSYVWRNIIPHVAPTHRCIAPDLIGMGKSDKPDLGYFFDDHVRFMDAFIEAL |
| | GLEEVVLVIHDWGSALGFHWAKRNPERVKGIAFMEFIRPIPTWDEWPEFARETFQAFRTT |
| | DVGRKLIIDQNVFIEGTLPMGVVRPLTEVEMDHYREPFLNPVDREPLWRFPNELPIAGEP |
| | ANIVALVEEYMDWLHQSPVPKLLFWGTPGVLIPPAEAARLAKSLPNCKAVDIGPGLNLLQ |
| | EDNPDLIGSEIARWLSTLEISGEPTTKSRITSEGEYIPLDQIDINVFCYENEV |
| | (SEQ ID NO: 109) |
| TsR (−) | MTDYEDHTIRRFRIFNAIMGGIHLLQVFLVLYLSNNFSLPVTISKPVYNEFTNSISPVSE |
| | TLFSIRVGPLVALFLFISAIAHILIATVLYYRYVENLKSGMNPYRWFEYSISASVMIVII |
| | AMLTTIYDLGTLLALFTLTAVMNLMGLMMELHNQTTQNTDWTSYIIGCIAGLVPWIVIFI |
| | PLIAAESVPDFVIYIFVSIAIFFNCFAINMYLQYKKIGKWKDYLHGERVYIILSLVAKSA |
| | LAWQVFAGTLRPMIGTGFPFDPHYVEVLGERMHYVDVGPRDGTPVLFLHGNPTSSYVWRN |
| | IIPHVAPTHRCIAPDLIGMGKSDKPDLGYFFDDHVRFMDAFIEALGLEEVVLVIHDWGSA |
| | LGFHWAKRNPERVKGIAFMEFIRPIPTWDEWPEFARETFQAFRTTDVGRKLIIDQNVFIE |
| | GTLPMGVVRPLTEVEMDHYREPFLNPVDREPLWRFPNELPIAGEPANIVALVEEYMDWLH |
| | QSPVPKLLFWGTPGVLIPPAEAARLAKSLPNCKAVDIGPGLNLLQEDNPDLIGSEIARWL |
| | STLEISGEPTTKSRITSEGEYIPLDQIDINVFCYENEV |
| | (SEQ ID NO: 110) |
| EhR (−) | MDTIVLTINWFNFIAGLFHVALAIVCALLGDVSQTFKMYMPFTTFRNGTQENEVTIKYSG |
| | YFPMTVIFVVYFSVTALFHMGNAFLWNDTYHRFLSNRKNPIRWTEYSITAPLMTAILAFI |
| | AGSRNWLFVIAASTLTFASISVGFVLDEIRKYSFFVLGMIPFLVEFSLIILSLYLTTSCY |
| | PSYLPITIFVEFGLWILFPFVSLLELSGINYKIGELVFIVLSFVSKSVLAIILNSENVLK |
| | NGVTGICIGTGFPFDPHYVEVLGERMHYVDVGPRDGTPVLFLHGNPTSSYVWRNIIPHVA |
| | PTHRCIAPDLIGMGKSDKPDLGYFFDDHVRFMDAFIEALGLEEVVLVIHDWGSALGFHWA |
| | KRNPERVKGIAFMEFIRPIPTWDEWPEFARETFQAFRTTDVGRKLIIDQNVFIEGTLPMG |
| | VVRPLTEVEMDHYREPFLNPVDREPLWRFPNELPIAGEPANIVALVEEYMDWLHQSPVPK |
| | LLFWGTPGVLIPPAEAARLAKSLPNCKAVDIGPGLNLLQEDNPDLIGSEIARWLSTLEIS |
| | GEPTTKSRITSEGEYIPLDQIDINVFCYENEV |
| | (SEQ ID NO: 111) |
| HaR (−) | MTKALEMTILDDGSESPISFKYLRKFNLAMGILHLVQGVAMLVLGFFWDFSRPLYSITLD |
| | YSGGPPVAALQPEFSFTAVGPTVAAFLLFSALAHLLIAGPLNKFYVKNLKKKMNPIRWFE |
| | YAFSSSIMVFFIAILFGVWDLWVLIGLFFLNMLMNLFGHMMELHNQTTKKTNWTAYIYGW |
| | IAGIIPWVIISVFFARIAINSTGMPWFVPVIYAFELILFMSFAFNMLLQYKKVGKWKDYL |
| | YGERMYQILSLVAKTLLAWLVFAGVFQPAIGTGFPFDPHYVEVLGERMHYVDVGPRDGTP |
| | VLFLHGNPTSSYVWRNIIPHVAPTHRCIAPDLIGMGKSDKPDLGYFFDDHVRFMDAFIEA |
| | LGLEEVVLVIHDWGSALGFHWAKRNPERVKGIAFMEFIRPIPTWDEWPEFARETFQAFRT |
| | TDVGRKLIIDQNVFIEGTLPMGVVRPLTEVEMDHYREPFLNPVDREPLWRFPNELPIAGE |
| | PANIVALVEEYMDWLHQSPVPKLLFWGTPGVLIPPAEAARLAKSLPNCKAVDIGPGLNLL |
| | QEDNPDLIGSEIARWLSTLEISGEPTTKSRITSEGEYIPLDQIDINVFCYENEV |
| | (SEQ ID NO: 112) |
| CR1 (+) | MPEPGSEAIWLWLGTAGMFLGMLYFIARGWGETDSRRQKFYIATILITAIAFVNYLAMAL |
| | GFGLTIVEFAGEEHPIYWARYSDWLFTTPLLLYNLGLLAGADRNTITSLVSLDVLMIGTG |
| | LVATLSPGSGVLSAGAERLVWWGISTAFLLVLLYFLFSSLSGRVADLPSDTRSTFKTLRN |
| | LVTVVWLVYPVWWLIGTEGIGLVGIGIVTAGFMVIDLTAKVGFGIILLRSHGVLDGAAIG |
| | TGFPFDPHYVEVLGERMHYVDVGPRDGTPVLFLHGNPTSSYVWRNIIPHVAPTHRCIAPD |
| | LIGMGKSDKPDLGYFFDDHVRFMDAFIEALGLEEVVLVIHDWGSALGFHWAKRNPERVKG |
| | IAFMEFIRPIPTWDEWPEFARETFQAFRTTDVGRKLIIDQNVFIEGTLPMGVVRPLTEVE |
| | MDHYREPFLNPVDREPLWRFPNELPIAGEPANIVALVEEYMDWLHQSPVPKLLFWGTPGV |
| | LIPPAEAARLAKSLPNCKAVDIGPGLNLLQEDNPDLIGSEIARWLSTLEISGEPTTKSRI |
| | TSEGEYIPLDQIDINVFCYENEV |
| | (SEQ ID NO: 113) |
| LmR (+) | MIVDQFEEVLMKTSQLFPLPTATQSAQPTHVAPVPTVLPDTPIYETVGDSGSKTLWVVFV |
| | LMLIASAAFTALSWKIPVNRRLYHVITTIITLTAALSYFAMATGHGVALNKIVIRTQHDH |
| | VPDTYETVYRQVYYARYIDWAITTPLLLLNLGLLAGMSGAHIFMAIVADLIMVLTGLFAA |
| | FGSEGTPQKWGWYTIACIAYIFVVWHLVLNGGANARVKGEKLRSFFVAIGAYTLILWTAY |
| | PIVWGLADGARKIGVDGVIIAYAVLDVLAKGVFGAWLLVTHANLRESDIGTGFPFDPHYV |
| | EVLGERMHYVDVGPRDGTPVLFLHGNPTSSYVWRNIIPHVAPTHRCIAPDLIGMGKSDKP |
| | DLGYFFDDHVRFMDAFIEALGLEEVVLVIHDWGSALGFHWAKRNPERVKGIAFMEFIRPI |
| | PTWDEWPEFARETFQAFRTTDVGRKLIIDQNVFIEGTLPMGVVRPLTEVEMDHYREPFLN |
| | PVDREPLWRFPNELPIAGEPANIVALVEEYMDWLHQSPVPKLLFWGTPGVLIPPAEAARL |
| | AKSLPNCKAVDIGPGLNLLQEDNPDLIGSEIARWLSTLEISGEPTTKSRITSEGEYIPLD |
| | QIDINVFCYENEV |
| | (SEQ ID NO: 114) |
| PaR (+) | MIHHDQVAEMLYGYAGAAAAKPTHTDSPGPIPTVIPTPPQFQEIGETGHRTLWVVFALMV |
| | LSSGFFAFMSWNVPISKRLYHVITTLITITASLSYFAMASGHVTSFSCTPAKDHHKHVPD |
| | VGYTECRQVFWGRYVDWAITTPLLLLNLSLLAGIDGAHTLMAVIADVIMVLSGLFASQGE |
| | TATQRWGWYAIGCVSYLFVIWHVALHGARTVTAKGRGVTRLFSSLALFTFVLWTAYPIVW |
| | GIADGAHRTTVDTVILIYAVLDILAKPVFGLWLLFSHRSLAETNIGTGFPFDPHYVEVLG |
| | ERMHYVDVGPRDGTPVLFLHGNPTSSYVWRNIIPHVAPTHRCIAPDLIGMGKSDKPDLGY |
| | FFDDHVRFMDAFIEALGLEEVVLVIHDWGSALGFHWAKRNPERVKGIAFMEFIRPIPTWD |
| | EWPEFARETFQAFRTTDVGRKLIIDQNVFIEGTLPMGVVRPLTEVEMDHYREPFLNPVDR |
| | EPLWRFPNELPIAGEPANIVALVEEYMDWLHQSPVPKLLFWGTPGVLIPPAEAARLAKSL |
| | PNCKAVDIGPGLNLLQEDNPDLIGSEIARWLSTLEISGEPTTKSRITSEGEYIPLDQIDI |
| | NVFCYENEV |
| | (SEQ ID NO: 115) |
| NcR (+) | MIHPEQVADMLRPTTSTTSSHVPGPVPTVVPTPTEYQTLGETGHRTLWVTFALMVLSSGI |
| | FALLSWNVPTSKRLFHVITTLITVVASLSYFAMATGHATTENCDTAWDHHKHVPDTSHQV |
| | CRQVFWGRYVDWALTTPLLLLQLCLLAGVDGAHTLMAIVADVIMVLCGLFAALGEGGNTA |
| | QKWGWYTIGCFSYLFVIWHVALHGSRTVTAKGRGVSRLFTGLAVFALLLWTAYPIIWGIA |
| | GGARRTNVDTVILIYTVLDLLAKPVFGFWLLLSHRAMPETNIGTGFPFDPHYVEVLGERM |
| | HYVDVGPRDGTPVLFLHGNPTSSYVWRNIIPHVAPTHRCIAPDLIGMGKSDKPDLGYFFD |
| | DHVRFMDAFIEALGLEEVVLVIHDWGSALGFHWAKRNPERVKGIAFMEFIRPIPTWDEWP |
| | EFARETFQAFRTTDVGRKLIIDQNVFIEGTLPMGVVRPLTEVEMDHYREPFLNPVDREPL |
| | WRFPNELPIAGEPANIVALVEEYMDWLHQSPVPKLLFWGTPGVLIPPAEAARLAKSLPNC |
| | KAVDIGPGLNLLQEDNPDLIGSEIARWLSTLEISGEPTTKSRITSEGEYIPLDQIDINVF |
| | CYENEV |
| | (SEQ ID NO: 116) |
| Ace1 (+) | MSNPNPFQTTLGTDAQWVVFAVMALAAIVFSIAVQFRPLPLRLTYYVNIAICTIAATAYY |
| | AMAVNGGDNKPTAGTGADERQVIYARYIDWVFTTPLLLLNLVLLTNMPATMIAWIMGADI |
| | AMIAFGIIGAFTVGSYKWFYFVVGCIMLAVLAWGMINPIFKEELQKHKEYTGAYTTLLIY |
| | LIVLWVIYPIVWGLGAGGHIIGVDVVIIAMGVLDLLAKPLYAIGVLITVEVVYGKIGTGF |
| | PFDPHYVEVLGERMHYVDVGPRDGTPVLFLHGNPTSSYVWRNIIPHVAPTHRCIAPDLIG |
| | MGKSDKPDLGYFFDDHVRFMDAFIEALGLEEVVLVIHDWGSALGFHWAKRNPERVKGIAF |
| | MEFIRPIPTWDEWPEFARETFQAFRTTDVGRKLIIDQNVFIEGTLPMGVVRPLTEVEMDH |
| | YREPFLNPVDREPLWRFPNELPIAGEPANIVALVEEYMDWLHQSPVPKLLFWGTPGVLIP |
| | PAEAARLAKSLPNCKAVDIGPGLNLLQEDNPDLIGSEIARWLSTLEISGEPTTKSRITSE |
| | GEYIPLDQIDINVFCYENEV |
| | (SEQ ID NO: 117) |
| CvR (+) | MAVHQIGEGGLVMYWVTFGLMAFSALAFAVMTFTRPLNKRSHGYITLAIVTIAAIAYYAM |
| | AASGGKALVSNPDGNLRDIYYARYIDWFFTTPLLLLNIILLTGIPIGVTLWIVLADVAMI |
| | MLGLFGALSTNSYRWGYYGVSCAFFFVVLWGLFFPGAKGARARGGQVPGLYFGLAGYLAL |
| | LWFGYPIVWGLAEGSDYISVTAVAASYAGLDIAAKVVFGWAVMLSHPLIARNQIGTGFPF |
| | DPHYVEVLGERMHYVDVGPRDGTPVLFLHGNPTSSYVWRNIIPHVAPTHRCIAPDLIGMG |
| | KSDKPDLGYFFDDHVRFMDAFIEALGLEEVVLVIHDWGSALGFHWAKRNPERVKGIAFME |
| | FIRPIPTWDEWPEFARETFQAFRTTDVGRKLIIDQNVFIEGTLPMGVVRPLTEVEMDHYR |
| | EPFLNPVDREPLWRFPNELPIAGEPANIVALVEEYMDWLHQSPVPKLLFWGTPGVLIPPA |
| | EAARLAKSLPNCKAVDIGPGLNLLQEDNPDLIGSEIARWLSTLEISGEPTTKSRITSEGE |
| | YIPLDQIDINVFCYENEV |
| | (SEQ ID NO: 118) |
| KnR (+) | MVYSHADAAGWTLYWITYGIMAVTALIFFAMSLRRPIQQRSHHYTSFLIVAIASLAYYAM |
| | ASQGGNTRIRVYQGPADSYRQIFWARYVDWFFTTPLLLLNLVLLSNLSKLRIAAIMVADI |
| | FMILTGLFGAVEARSNKWGWFVFGCIFMLYIFYELLVNVRKGAYARGGQHGMLYSVLLVW |
| | LLILWVQYPVVWGLAEGSSTVSSDTVIAWYAALDICAKCVFGFILLLGIESIDRKRIGTG |
| | FPFDPHYVEVLGERMHYVDVGPRDGTPVLFLHGNPTSSYVWRNIIPHVAPTHRCIAPDLI |
| | GMGKSDKPDLGYFFDDHVRFMDAFIEALGLEEVVLVIHDWGSALGFHWAKRNPERVKGIA |
| | FMEFIRPIPTWDEWPEFARETFQAFRTTDVGRKLIIDQNVFIEGTLPMGVVRPLTEVEMD |
| | HYREPFLNPVDREPLWRFPNELPIAGEPANIVALVEEYMDWLHQSPVPKLLFWGTPGVLI |
| | PPAEAARLAKSLPNCKAVDIGPGLNLLQEDNPDLIGSEIARWLSTLEISGEPTTKSRITS |
| | EGEYIPLDQIDINVFCYENEV |
| | (SEQ ID NO: 119) |
| TdR (+) | MSSLFEKRNTAVATNYRGPTNSIVINEAGSDWYWAVFSVMAASAIVFSVMAAMTPRGERV |
| | FHYLTIAIVSVASVAYFTMAADLGSVAIISEFANYSSLPTRQVFYARYIDWVITTPLLLT |
| | NLMLLAGLPWSTIIFTIVMDEVMVLTGLFGAITPSSYKWGYFTFGMVAYFFVAWVLIVEA |
| | RKNAHRLGSDVHRLYIGIAIWTATLWTLYPVAWGLSEGGNVTSSDGVAIFYGVLDLLAKP |
| | VFGLWILLGHKGIGMDRIGTGFPFDPHYVEVLGERMHYVDVGPRDGTPVLFLHGNPTSSY |
| | VWRNIIPHVAPTHRCIAPDLIGMGKSDKPDLGYFFDDHVRFMDAFIEALGLEEVVLVIHD |
| | WGSALGFHWAKRNPERVKGIAFMEFIRPIPTWDEWPEFARETFQAFRTTDVGRKLIIDQN |
| | VFIEGTLPMGVVRPLTEVEMDHYREPFLNPVDREPLWRFPNELPIAGEPANIVALVEEYM |
| | DWLHQSPVPKLLFWGTPGVLIPPAEAARLAKSLPNCKAVDIGPGLNLLQEDNPDLIGSEI |
| | ARWLSTLEISGEPTTKSRITSEGEYIPLDQIDINVFCYENEV |
| | (SEQ ID NO: 120) |
| EsR (+) | MESNLVRRNGALTVNTMTQNNQSVAIHQTVRGSDWYYTVCAVMGTTSLAILALSRMKPRT |
| | DRVFFYLTSGLCMVACIAYFAMGSNLGWTPIDVEWLRNDSVVRGVNRQVFYARYIDWVIT |
| | TPMLLMNLLLTAGMPWPTILWIILLDEIMIVTGLIGALVKSRYKWGFYVFGCMAMFYIMW |
| | ELAFPARKHAKVLGKDIHRSFVLCGVLTLVVWLCYPICWGLSEGGNVISPDSVSVFYGVL |
| | DVLAKPSFSIALIATHWNIDPGRIGTGFPFDPHYVEVLGERMHYVDVGPRDGTPVLFLHG |
| | NPTSSYVWRNIIPHVAPTHRCIAPDLIGMGKSDKPDLGYFFDDHVRFMDAFIEALGLEEV |
| | VLVIHDWGSALGFHWAKRNPERVKGIAFMEFIRPIPTWDEWPEFARETFQAFRTTDVGRK |
| | LIIDQNVFIEGTLPMGVVRPLTEVEMDHYREPFLNPVDREPLWRFPNELPIAGEPANIVA |
| | LVEEYMDWLHQSPVPKLLFWGTPGVLIPPAEAARLAKSLPNCKAVDIGPGLNLLQEDNPD |
| | LIGSEIARWLSTLEISGEPTTKSRITSEGEYIPLDQIDINVFCYENEV |
| | (SEQ ID NO: 121) |
| MIR (+) | MGNSAIEVNGDTVDSYTADVKITTHGSDAYWAITAVMAFTTILFIAHSFTKPRTDRIFHY |
| | ITISITLVASIAYFTMASNLGWASIFIEFQRDDPLVSGTTREIFYVRYIDWVITTPLLLL |
| | NILLTAGLPWPTILFTILLDEVMIITGLVGALVKSSYKWGFFTFGCVAFFGVAWSVAWTG |
| | RKHANALGSDIGRVYLMTSVWTLFLWLLYPIAWGVSEGGNVISPDSVAAFYGTLDVLAKP |
| | IFGIILLWGHRNIPASRIGTGFPFDPHYVEVLGERMHYVDVGPRDGTPVLFLHGNPTSSY |
| | VWRNIIPHVAPTHRCIAPDLIGMGKSDKPDLGYFFDDHVRFMDAFIEALGLEEVVLVIHD |
| | WGSALGFHWAKRNPERVKGIAFMEFIRPIPTWDEWPEFARETFQAFRTTDVGRKLIIDQN |
| | VFIEGTLPMGVVRPLTEVEMDHYREPFLNPVDREPLWRFPNELPIAGEPANIVALVEEYM |
| | DWLHQSPVPKLLFWGTPGVLIPPAEAARLAKSLPNCKAVDIGPGLNLLQEDNPDLIGSEI |
| | ARWLSTLEISGEPTTKSRITSEGEYIPLDQIDINVFCYENEV |
| | (SEQ ID NO: 122) |
| ScR (+) | MDSLLLQRRNDAVATNPPNATFALTENGSSWLWAVFCVMALSCIIIAALSLFKPMGYRIF |
| | YLLNVAILATASVSYFSLASDLGLTPVTVEFRGPGTRQIAYVRYIDWFVTTPLLLTQLLL |
| | TAGLPTNIIISTIFADLVMIITGLAGALVVSRYKWGYYTMGCVAMLWVFWNVFTGIKVSG |
| | NIGPDVRKSYTLLAVWLMIIWLNYPICWGLAEGGNRITVVGVMVYYGVLDLLAKPVFAAI |
| | SLAVHSKIELSRIGTGFPFDPHYVEVLGERMHYVDVGPRDGTPVLFLHGNPTSSYVWRNI |
| | IPHVAPTHRCIAPDLIGMGKSDKPDLGYFFDDHVRFMDAFIEALGLEEVVLVIHDWGSAL |
| | GFHWAKRNPERVKGIAFMEFIRPIPTWDEWPEFARETFQAFRTTDVGRKLIIDQNVFIEG |
| | TLPMGVVRPLTEVEMDHYREPFLNPVDREPLWRFPNELPIAGEPANIVALVEEYMDWLHQ |
| | SPVPKLLFWGTPGVLIPPAEAARLAKSLPNCKAVDIGPGLNLLQEDNPDLIGSEIARWLS |
| | TLEISGEPTTKSRITSEGEYIPLDQIDINVFCYENEV |
| | (SEQ ID NO: 123) |
| RgR (+) | MDAILSKRNEVLSLNPLVANIDITTAASDWLWAVFAVMGLSAIILLVLGHATRPIGERAF |
| | HELAAALCFTASIAYYSMASDLGATPIEVEFIRGGTLGQNWVDIGVLRPTRSIWYARYID |
| | WTITTPLLLLQLALTTALPLSQIFGLVFFDIVMIITGLLGALTASRYKWGFFVFGCVAMF |
| | WIFWVLFFPARKSASHLGTDYHRAYTSSAIVLCTLWTVYPIIWGVCDGGNVITPTSVMVA |
| | YGVLDLLAKPVFSFWHVFQLSRLDYARIGTGFPFDPHYVEVLGERMHYVDVGPRDGTPVL |
| | FLHGNPTSSYVWRNIIPHVAPTHRCIAPDLIGMGKSDKPDLGYFFDDHVRFMDAFIEALG |
| | LEEVVLVIHDWGSALGFHWAKRNPERVKGIAFMEFIRPIPTWDEWPEFARETFQAFRTTD |
| | VGRKLIIDQNVFIEGTLPMGVVRPLTEVEMDHYREPFLNPVDREPLWRFPNELPIAGEPA |
| | NIVALVEEYMDWLHQSPVPKLLFWGTPGVLIPPAEAARLAKSLPNCKAVDIGPGLNLLQE |
| | DNPDLIGSEIARWLSTLEISGEPTTKSRITSEGEYIPLDQIDINVFCYENEV |
| | (SEQ ID NO: 124) |
| RtR (+) | MPFYIKNADIDITTHGSDWLWAVFSVMLLSAIGILVWGHVARPLGERAFHELAAALCFTA |
| | SIAYFAMASDLGDVPIVVEFIRGGSLGQNWVQVGVENPTRAIWYARYIDWTITTPMLLLQ |
| | LLLCTGLPLSQVFSVIFADLLMIETGLIGALVASRYKWGFYAFGCAAQLYIWWMLLVPGR |
| | RSAQHIGSDFAKSYTMSNIFLTTVWLVYPVIWGVADGGNVITPDSVMIAYGVLDLLAKPV |
| | FSVIHLMSLSKLDYARIGTGFPFDPHYVEVLGERMHYVDVGPRDGTPVLFLHGNPTSSYV |
| | WRNIIPHVAPTHRCIAPDLIGMGKSDKPDLGYFFDDHVRFMDAFIEALGLEEVVLVIHDW |
| | GSALGFHWAKRNPERVKGIAFMEFIRPIPTWDEWPEFARETFQAFRTTDVGRKLIIDQNV |
| | FIEGTLPMGVVRPLTEVEMDHYREPFLNPVDREPLWRFPNELPIAGEPANIVALVEEYMD |
| | WLHQSPVPKLLFWGTPGVLIPPAEAARLAKSLPNCKAVDIGPGLNLLQEDNPDLIGSEIA |
| | RWLSTLEISGEPTTKSRITSEGEYIPLDQIDINVFCYENEV |
| | (SEQ ID NO: 125) |
| KeR1 (+) | MKFLLLLLADPTKLDPSDYVGFTFFVGAMAMMAASAFFFLSLNQFNKKWRTSVLVSGLIT |
| | FIAAVHYWYMRDYWFAIQESPTFFRYVDWVLTVPLMCVQFYLILKVAGAKPALMWKLILF |
| | SVIMLVTGYFGEAVFQDQAALWGAISGAAYFYIVYEIWLGSAKKLAVAAGGDILKAHKIL |
| | CWFVLVGWAIYPLGYMLGTDGWYTSILGKGSVDVAYNIADAINKIGFGLVIYALAVKKNE |
| | VDIGTGFPFDPHYVEVLGERMHYVDVGPRDGTPVLFLHGNPTSSYVWRNIIPHVAPTHRC |
| | IAPDLIGMGKSDKPDLGYFFDDHVRFMDAFIEALGLEEVVLVIHDWGSALGFHWAKRNPE |
| | RVKGIAFMEFIRPIPTWDEWPEFARETFQAFRTTDVGRKLIIDQNVFIEGTLPMGVVRPL |
| | TEVEMDHYREPFLNPVDREPLWRFPNELPIAGEPANIVALVEEYMDWLHQSPVPKLLFWG |
| | TPGVLIPPAEAARLAKSLPNCKAVDIGPGLNLLQEDNPDLIGSEIARWLSTLEISGEPTT |
| | KSRITSEGEYIPLDQIDINVFCYENEV |
| | (SEQ ID NO: 126) |
| KeR2 (+) | MTQELGNANFENFIGATEGFSEIAYQFTSHILTLGYAVMLAGLLYFILTIKNVDKKFQMS |
| | NILSAVVMVSAFLLLYAQAQNWTSSFTFNEEVGRYFLDPSGDLFNNGYRYLNWLIDVPML |
| | LFQILFVVSLTTSKFSSVRNQFWFSGAMMIITGYIGQFYEVSNLTAFLVWGAISSAFFFH |
| | ILWVMKKVINEGKEGISPAGQKILSNIWILFLISWTLYPGAYLMPYLTGVDGFLYSEDGV |
| | MARQLVYTIANVSSKVIYGVLLGNLAITLSKNKIGTGFPFDPHYVEVLGERMHYVDVGPR |
| | DGTPVLFLHGNPTSSYVWRNIIPHVAPTHRCIAPDLIGMGKSDKPDLGYFFDDHVRFMDA |
| | FIEALGLEEVVLVIHDWGSALGFHWAKRNPERVKGIAFMEFIRPIPTWDEWPEFARETFQ |
| | AFRTTDVGRKLIIDQNVFIEGTLPMGVVRPLTEVEMDHYREPFLNPVDREPLWRFPNELP |
| | IAGEPANIVALVEEYMDWLHQSPVPKLLFWGTPGVLIPPAEAARLAKSLPNCKAVDIGPG |
| | LNLLQEDNPDLIGSEIARWLSTLEISGEPTTKSRITSEGEYIPLDQIDINVFCYENEV |
| | (SEQ ID NO: 127) |
| GvR (+) | MLMTVFSSAPELALLGSTFAQVDPSNLSVSDSLTYGQFNLVYNAFSFAIAAMFASALFFF |
| | SAQALVGQRYRLALLVSAIVVSIAGYHYFRIFNSWDAAYVLENGVYSLTSEKFNDAYRYV |
| | DWLLTVPLLLVQTVAVLTLPAKEARPLLIKLTVASVLMIATGYPGEISDDITTRIIWGTV |
| | STIPFAYILYVLWVELSRSLVRQPAAVQTLVRNMRWLLLLSWGVYPIAYLLPMLGVSGTS |
| | AAVGVQVGYTIADVLAKPVFGLLVFAIALVKTKADIGTGFPFDPHYVEVLGERMHYVDVG |
| | PRDGTPVLFLHGNPTSSYVWRNIIPHVAPTHRCIAPDLIGMGKSDKPDLGYFFDDHVRFM |
| | DAFIEALGLEEVVLVIHDWGSALGFHWAKRNPERVKGIAFMEFIRPIPTWDEWPEFARET |
| | FQAFRTTDVGRKLIIDQNVFIEGTLPMGVVRPLTEVEMDHYREPFLNPVDREPLWRFPNE |
| | LPIAGEPANIVALVEEYMDWLHQSPVPKLLFWGTPGVLIPPAEAARLAKSLPNCKAVDIG |
| | PGLNLLQEDNPDLIGSEIARWLSTLEISGEPTTKSRITSEGEYIPLDQIDINVFCYENEV |
| | (SEQ ID NO: 128) |
| SrR (+) | MLQELPTLTPGQYSLVFNMFSFTVATMTASFVFFVLARNNVAPKYRISMMVSALVVFIAG |
| | YHYFRITSSWEAAYALQNGMYQPTGELFNDAYRYVDWLLTVPLLTVQLVLVMGLPKNERG |
| | PLAAKLGFLAALMIVLGYPGEVSENAALFGTRGLWGFLSTIPFVWILYILFTQLGDTIQR |
| | QSSRVSTLLGNARLLLLATWGFYPIAYMIPMAFPEAFPSNTPGTIVALQVGYTIADVLAK |
| | AGYGVLIYNIAKAKSEEEIGTGFPFDPHYVEVLGERMHYVDVGPRDGTPVLFLHGNPTSS |
| | YVWRNIIPHVAPTHRCIAPDLIGMGKSDKPDLGYFFDDHVRFMDAFIEALGLEEVVLVIH |
| | DWGSALGFHWAKRNPERVKGIAFMEFIRPIPTWDEWPEFARETFQAFRTTDVGRKLIIDQ |
| | NVFIEGTLPMGVVRPLTEVEMDHYREPFLNPVDREPLWRFPNELPIAGEPANIVALVEEY |
| | MDWLHQSPVPKLLFWGTPGVLIPPAEAARLAKSLPNCKAVDIGPGLNLLQEDNPDLIGSE |
| | IARWLSTLEISGEPTTKSRITSEGEYIPLDQIDINVFCYENEV |
| | (SEQ ID NO: 129) |
| PsuCCR (+) | MTMLEHLEGTMDGWYAENDLGQGAIIAHWVTFFFHMITTFYLGYVSFHSKGPGGKQPYFA |
| | GYHEENNIGIFVNLFAAISYFGKVVSDTHGHNYQNVGPFIIGLGNYRYADYMLTCPLLVM |
| | NLLFQLRAPYKITCAMLIFAVLMIGAVTNFYPGDDMKGPAVAWFCFGCFWYLIAYIFMAH |
| | IVSKQYGRLDYLAHGTKAEGALFSLKLAIITFFAIWVAFPLVWLLSVGTGVLSNEAAEIC |
| | HCICDVVAKSVYGFALANFREQYDRELIGTGFPFDPHYVEVLGERMHYVDVGPRDGTPVL |
| | FLHGNPTSSYVWRNIIPHVAPTHRCIAPDLIGMGKSDKPDLGYFFDDHVRFMDAFIEALG |
| | LEEVVLVIHDWGSALGFHWAKRNPERVKGIAFMEFIRPIPTWDEWPEFARETFQAFRTTD |
| | VGRKLIIDQNVFIEGTLPMGVVRPLTEVEMDHYREPFLNPVDREPLWRFPNELPIAGEPA |
| | NIVALVEEYMDWLHQSPVPKLLFWGTPGVLIPPAEAARLAKSLPNCKAVDIGPGLNLLQE |
| | DNPDLIGSEIARWLSTLEISGEPTTKSRITSEGEYIPLDQIDINVFCYENEV |
| | (SEQ ID NO: 130) |
| GtCCR1 (+) | MVESSAVIAANWISFLVIAGSFVVLCFISLRYKGPGGNENYYNGFREQNMLTVIINLWCA |
| | LAYFAKVLQSHSDDDGFVPLTKIPYLDYATTCPLLTLNLMWCLDAPYKITSAVLVFTVMI |
| | TGVACSLAVAPYSFYWFAMGMVLFIFTYVLMLSIVRERLEFITQCAHDSNAKRSIKHLKA |
| | AVIIYFGIWPIFAILWLLSYRAANVISNDTNHILHCILDVIAKSCFGFVLLHFKMYFDKK |
| | LIGTGFPFDPHYVEVLGERMHYVDVGPRDGTPVLFLHGNPTSSYVWRNIIPHVAPTHRCI |
| | APDLIGMGKSDKPDLGYFFDDHVRFMDAFIEALGLEEVVLVIHDWGSALGFHWAKRNPER |
| | VKGIAFMEFIRPIPTWDEWPEFARETFQAFRTTDVGRKLIIDQNVFIEGTLPMGVVRPLT |
| | EVEMDHYREPFLNPVDREPLWRFPNELPIAGEPANIVALVEEYMDWLHQSPVPKLLFWGT |
| | PGVLIPPAEAARLAKSLPNCKAVDIGPGLNLLQEDNPDLIGSEIARWLSTLEISGEPTTK |
| | SRITSEGEYIPLDQIDINVFCYENEV |
| | (SEQ ID NO: 131) |
| GtCCR2 (+) | MVASSAVITANWISFLAISASFIILLVISLRYKGPGGTESFYNGFKEQNMLTVFINLWCA |
| | LAYFAKVLQSHSNDNGFAPLTVIPYVDYCTTCPLLTLNLLWCLDAPYKISSAVLVFTCLV |
| | IAVACSLAVAPFSYCWFAMGMVLFTFTYVFILSIVRQRLDFFTLCARDSNAKQSLKHLKT |
| | AVFIYFGIWLLFPLLWLLSYRAANVISNDINHIFHCILDVIAKSVYGFALLYFKMYFDKK |
| | LIGTGFPFDPHYVEVLGERMHYVDVGPRDGTPVLFLHGNPTSSYVWRNIIPHVAPTHRCI |
| | APDLIGMGKSDKPDLGYFFDDHVRFMDAFIEALGLEEVVLVIHDWGSALGFHWAKRNPER |
| | VKGIAFMEFIRPIPTWDEWPEFARETFQAFRTTDVGRKLIIDQNVFIEGTLPMGVVRPLT |
| | EVEMDHYREPFLNPVDREPLWRFPNELPIAGEPANIVALVEEYMDWLHQSPVPKLLFWGT |
| | PGVLIPPAEAARLAKSLPNCKAVDIGPGLNLLQEDNPDLIGSEIARWLSTLEISGEPTTK |
| | SRITSEGEYIPLDQIDINVFCYENEV |
| | (SEQ ID NO: 132) |
| GtCCR3 (+) | MVSALDQNGPQYLQNPIVIAADWIGFIALFGSSLAVAYKLVTFKGPDQDDVYFFGYREEK |
| | MISVFVNLFAALAYWAKLASHANGDVGPAASVTTYKYLDYLFTCPLLTINLLWCLNLPYK |
| | FTFGAIVAVCILCAFMASVIPPPARYMWFGMGITVFSAAWFNILKLVRMRLEQFVSKEAK |
| | KVRQSLKVACMTYFFIWLGYPTLWVLGDAGVLDSVVSALLHTFLDVFSKSIYGFALLHFV |
| | MRTDKREIGTGFPFDPHYVEVLGERMHYVDVGPRDGTPVLFLHGNPTSSYVWRNIIPHVA |
| | PTHRCIAPDLIGMGKSDKPDLGYFFDDHVRFMDAFIEALGLEEVVLVIHDWGSALGFHWA |
| | KRNPERVKGIAFMEFIRPIPTWDEWPEFARETFQAFRTTDVGRKLIIDQNVFIEGTLPMG |
| | VVRPLTEVEMDHYREPFLNPVDREPLWRFPNELPIAGEPANIVALVEEYMDWLHQSPVPK |
| | LLFWGTPGVLIPPAEAARLAKSLPNCKAVDIGPGLNLLQEDNPDLIGSEIARWLSTLEIS |
| | GEPTTKSRITSEGEYIPLDQIDINVFCYENEV |
| | (SEQ ID NO: 133) |
| GtCCR4 (+) | MTTSAPSLSDPNWQYGMGGWNNPRLPNFNLHDPTVIGVDWLGFLCLLGASLALMYKLMSF |
| | KGPDGDQEFFVGYREEKCLSIYVNLIAAITYWGRICAHFNNDMGLSLSVNYFKYLDYIFT |
| | CPILTLNLLWSLNLPYKITYSLFVGLTIACNAFEPPARYLWFMFGCFIFAFTWISIIRLV |
| | YARFQQFLNEDAKKIRAPLKLSLTLYFSIWCGYPALWLLTEFGAISQLAAHVMTVIMDVA |
| | AKSVYGFALLKFQLGVDKRDIGTGFPFDPHYVEVLGERMHYVDVGPRDGTPVLFLHGNPT |
| | SSYVWRNIIPHVAPTHRCIAPDLIGMGKSDKPDLGYFFDDHVRFMDAFIEALGLEEVVLV |
| | IHDWGSALGFHWAKRNPERVKGIAFMEFIRPIPTWDEWPEFARETFQAFRTTDVGRKLII |
| | DQNVFIEGTLPMGVVRPLTEVEMDHYREPFLNPVDREPLWRFPNELPIAGEPANIVALVE |
| | EYMDWLHQSPVPKLLFWGTPGVLIPPAEAARLAKSLPNCKAVDIGPGLNLLQEDNPDLIG |
| | SEIARWLSTLEISGEPTTKSRITSEGEYIPLDQIDINVFCYENEV |
| | (SEQ ID NO: 134) |
| GtCCR5 (+) | MSTSSVAYLRTPVVQALDWVGFISLGGTAAVLAYRLMNFKPPNKDILYFFGYREKGMISL |
| | YVNLFAAVAYYARITSHLSGDVGAATNIILYKYFDYLITCPLLTFNLLTTLNLPYKITYA |
| | VYVQITIFTGFMSANTPPPATFLWFAFGMLLFSYTWFNIISLVQVRFIQYFAKKGNTTQS |
| | RRVSVASKAGFRNKNVRNPLQTALSTYFCIWMVYPVLWLLLKTKVIDQVTEHCINVVMDV |
| | LAKSMYGFALLRFQLLMDKANIGTGFPFDPHYVEVLGERMHYVDVGPRDGTPVLFLHGNP |
| | TSSYVWRNIIPHVAPTHRCIAPDLIGMGKSDKPDLGYFFDDHVRFMDAFIEALGLEEVVL |
| | VIHDWGSALGFHWAKRNPERVKGIAFMEFIRPIPTWDEWPEFARETFQAFRTTDVGRKLI |
| | IDQNVFIEGTLPMGVVRPLTEVEMDHYREPFLNPVDREPLWRFPNELPIAGEPANIVALV |
| | EEYMDWLHQSPVPKLLFWGTPGVLIPPAEAARLAKSLPNCKAVDIGPGLNLLQEDNPDLI |
| | GSEIARWLSTLEISGEPTTKSRITSEGEYIPLDQIDINVFCYENEV |
| | (SEQ ID NO: 135) |

### Example 2—In Vitro Characterization of New Rhodopsin GEVIs

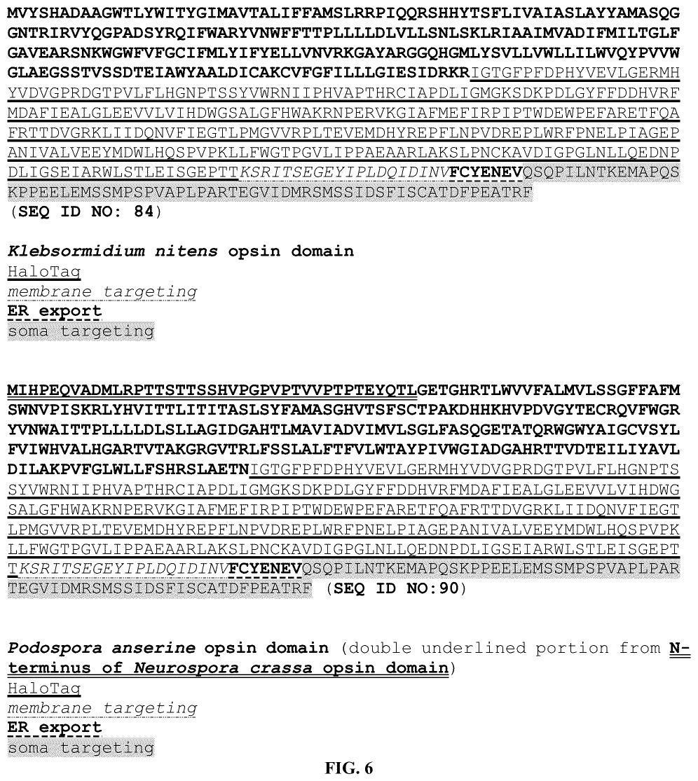

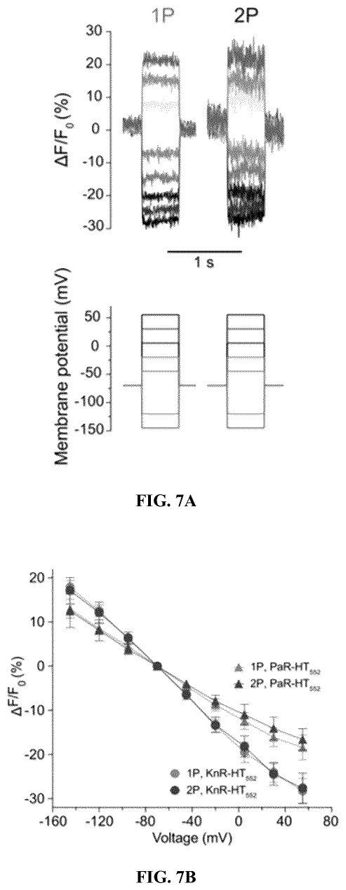

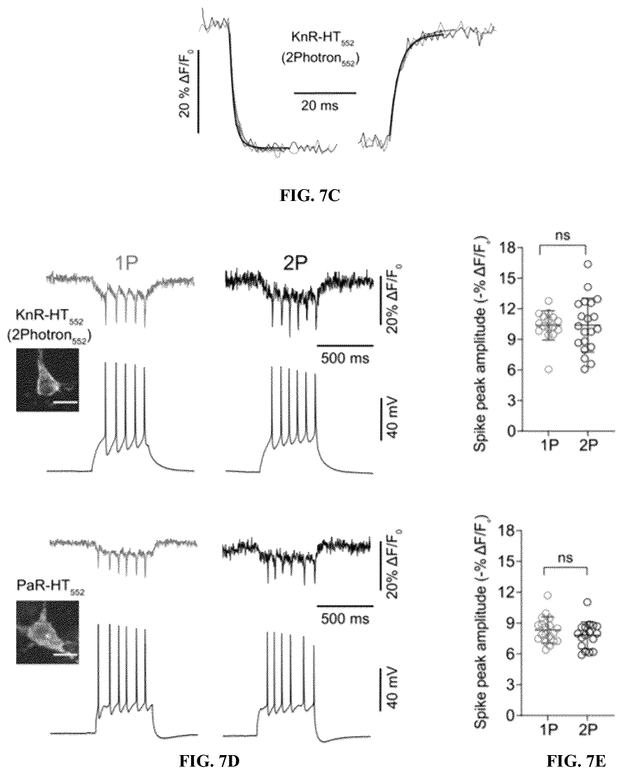

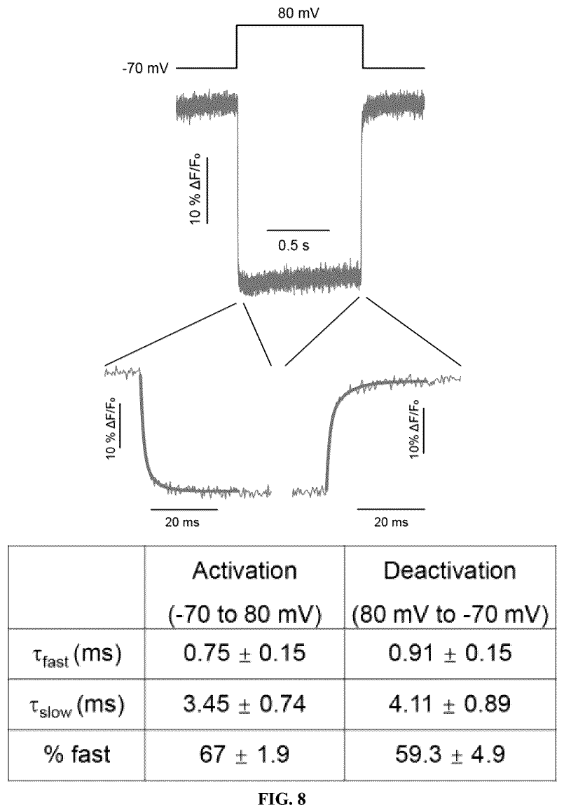

To characterize the new rhodopsin GEVIs, a microscope was adapted to allow sequential imaging of the same cell under 1P and 2P illumination with simultaneous patch electrophysiology to record or stimulate. Unlike conventional 2P point scanning illumination, an enlarged stationary focal spot was created to cover a significant part of a neuron cell body (details in Methods). To quantify the voltage sensitivity, cultured rat neurons expressing KnR-HT₅₅₂ or PaR-HT₅₅₂ were held at −70 mV and then stepped to different membrane potentials using a patch electrode under sequential 1P and 2P illumination (FIG. 7A). Both KnR-HT₅₅₂ and PaR-HT₅₅₂ exhibited nearly identical fluorescence changes in response to voltage steps under 1P and 2P illumination (FIG. 7A-7B). KnR-HT₅₅₂ showed higher voltage sensitivity than PaR-HT₅₅₂, giving a fluorescence change of about −24% ΔF/F₀ (−24±2.3% for 1P, −24.5±2.5% for 2P) for a voltage step from −70 mV to +30 mV. Fluorescence response kinetics were also similar under 1P and 2P illumination (FIG. 7C). KnR-HT₅₅₂ displayed fast voltage response with sub-millisecond on and off fast time constants (FIG. 8). Both KnR-HT₅₅₂ and PaR-HT₅₅₂ faithfully reported action potentials as well as subthreshold voltage signals with similar fluorescence response under 1P and 2P illumination (FIG. 7D-7E).

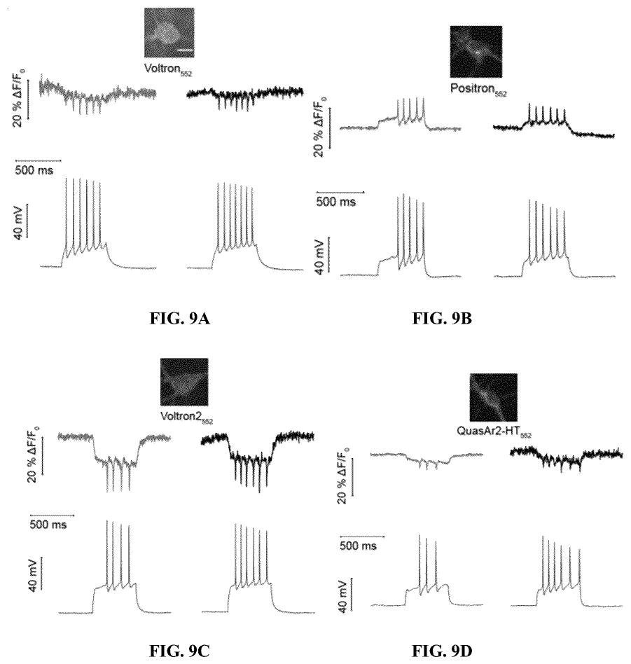

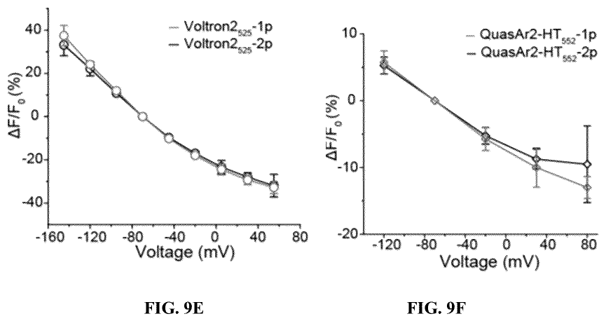

Existing rhodopsin-based voltage indicators, including Voltron, Voltron2, Positron2, and QuasAr2-HT, also exhibited similar voltage sensitivity with 1P and 2P illumination under these conditions (FIG. 9A-9F). To explore additional 2P illumination configurations, 1 kHz circular point scanning of neuron cell bodies expressing KnR-HT or Voltron2 was performed. Both KnR-HT and Voltron2 reported sub- and supra-threshold voltage changes in neurons with circular point scanning 2P excitation, with similar voltage sensitivity to 1P illumination (FIG. 10A-10B). Collectively, rhodopsin GEVIs are generally compatible with 2P imaging, at least under some illumination conditions. KnR-HT exhibited the most reliable responses under all tested 2P illumination conditions, so this protein was named 2Photron and characterized its performance further in vivo.

### Example 3-2P Voltage Recording with 2Photron in Awake, Behaving Mice

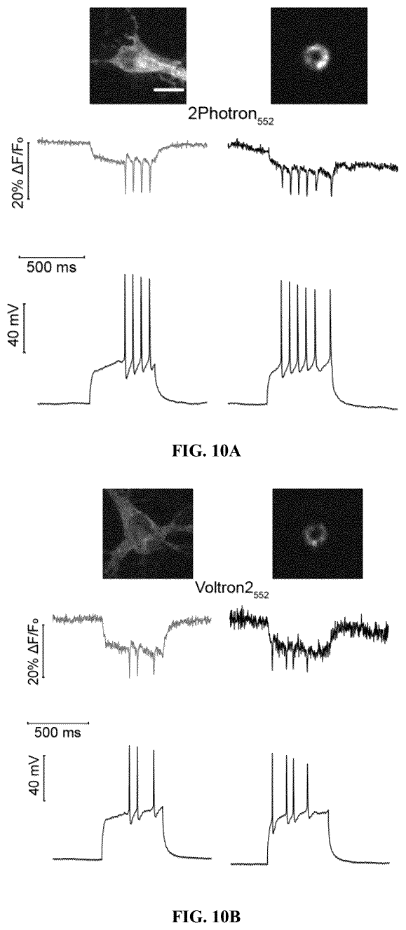

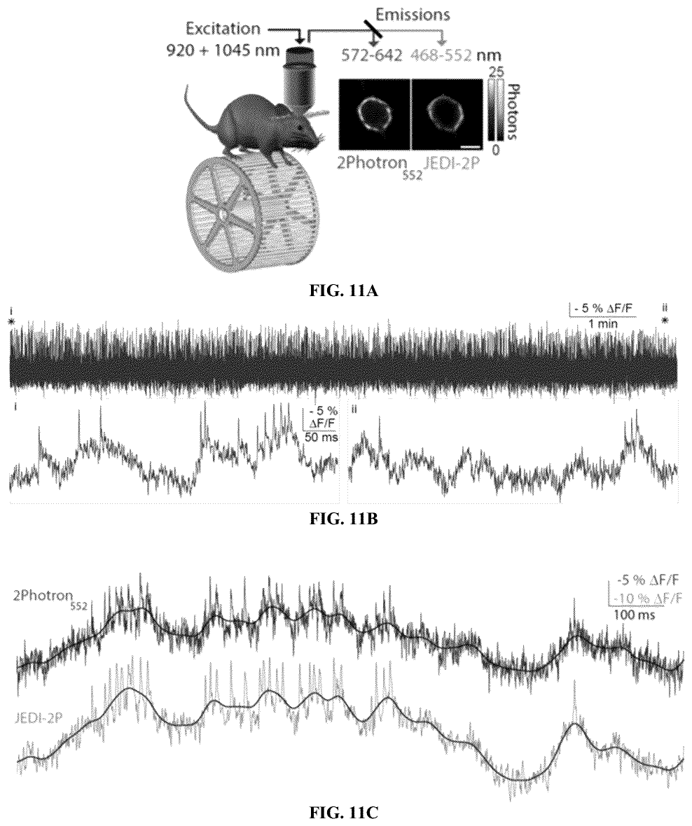

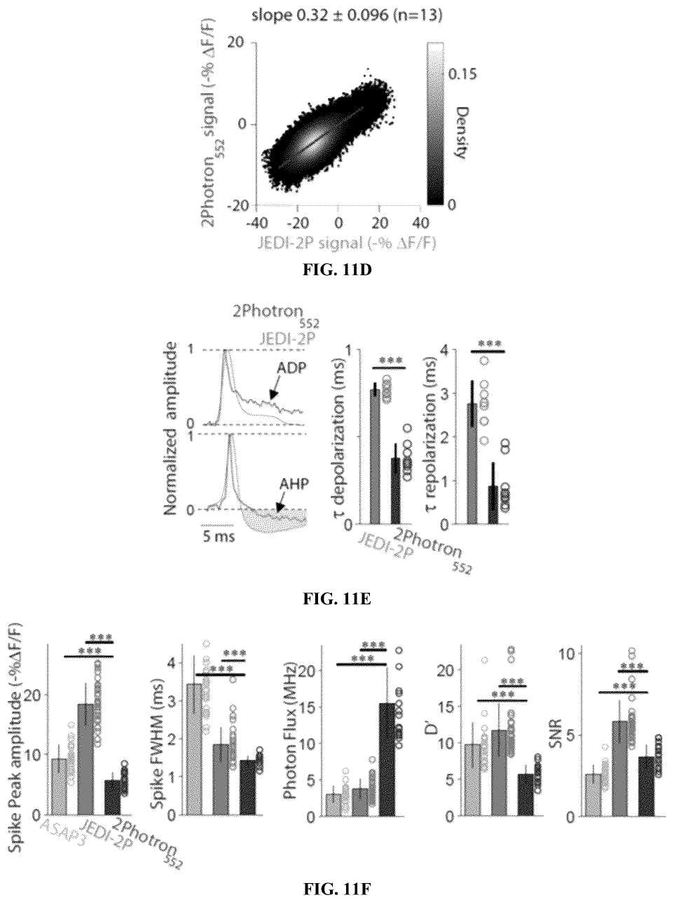

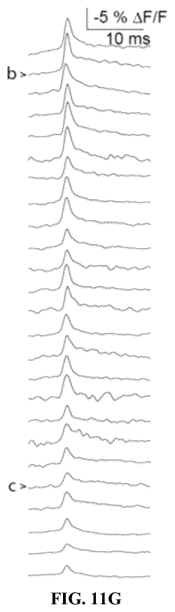

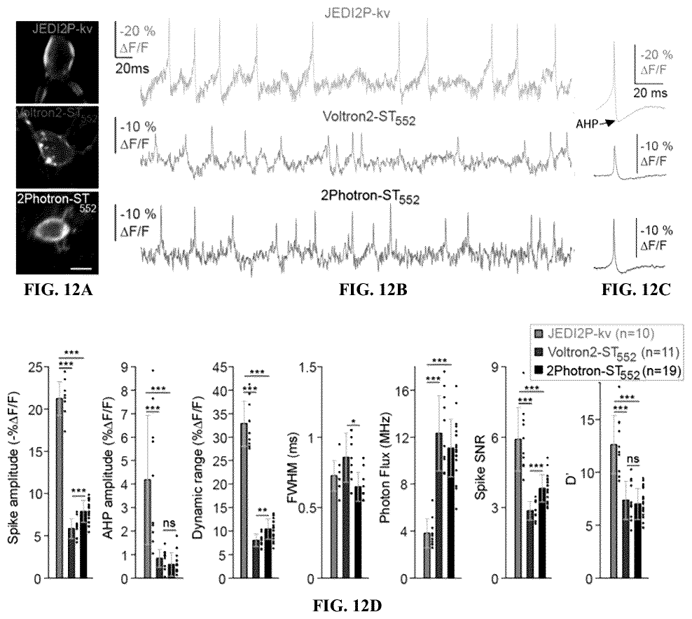

To explore whether voltage signals could be recorded in vivo using 2P excitation, ultrafast local volume excitation (ULoVE) optical recording was used to record from individual neurons of head-fixed, awake mice free to move on a circular treadmill (FIG. 11A). Adeno-associated virus (AAVs) was injected to express soma-targeted 2Photron (2Photron-ST) in mouse cortex or cerebellum. Bright, membrane-localized fluorescence of 2Photron was observed following intravenous injection of JF552-HTL (FIG. 11A). Using ULoVE with excitation at 1045 nm and >3.5 KHz sampling rate recording occurred from 25 cells in layer II/III of the visual cortex and 19 cerebellar glycinergic cells across 18 mice. Clear subthreshold fluctuations and negative-going spikes were observed in single trial data with 2Photron-ST₅₅₂, and could routinely record from individual neurons in layer II/III of visual cortex for ten minutes or more in awake mice with a mean ΔF/F for spikes of 5.72±1.39% (FIG. 11B). In fast-spiking cerebellar granular layer interneurons, 2Photron-ST₅₅₂ responded to spikes with a ΔF/F of 7.88±1.31% (FIG. 12A-12D).

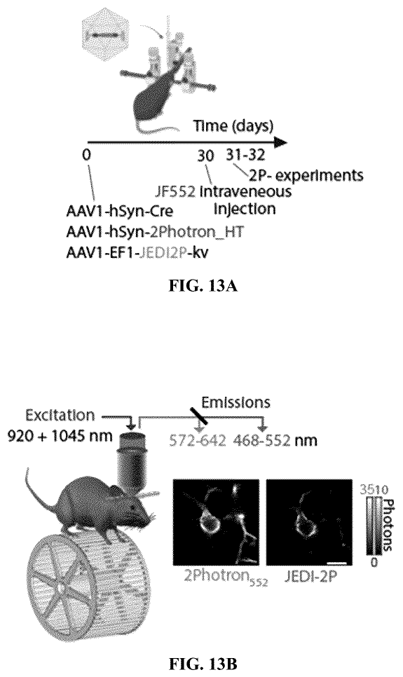

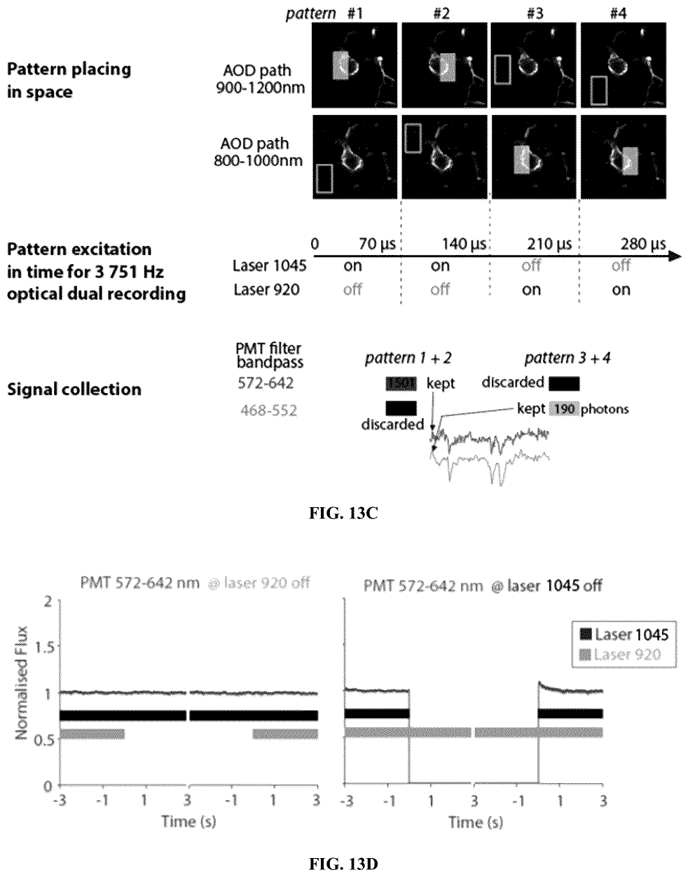

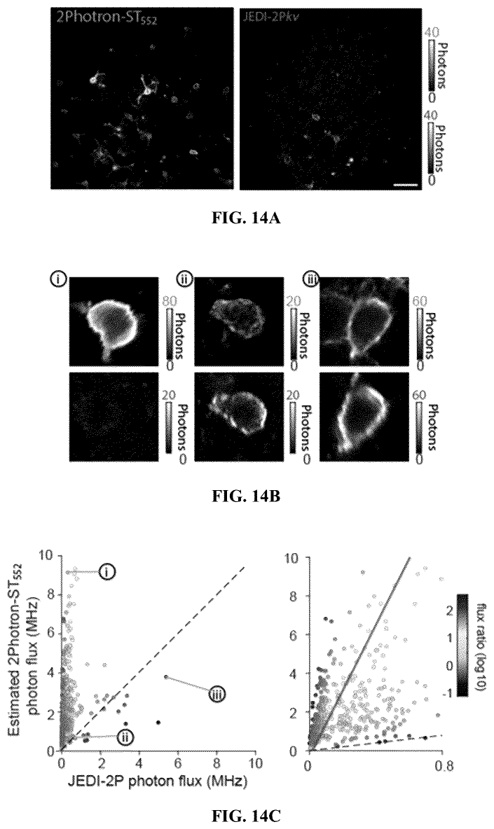

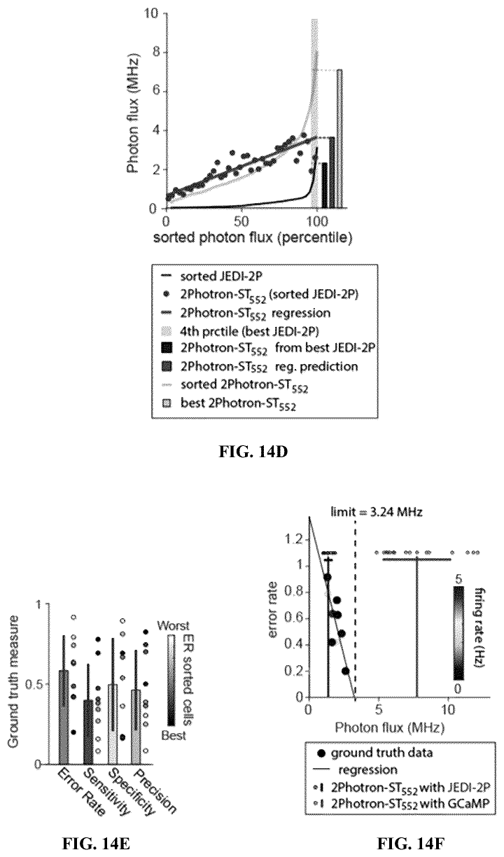

Using ULoVE with a dual-excitation path configuration¹³ (FIG. 13A-13D), simultaneous recording is possible from 2Photron-ST₅₅₂ and the GFP-based JEDI-2P-Kv¹⁴ in the same layer II/III visual cortical neurons following co-expression (FIG. 11A). This allowed for direct comparison of 2Photron-ST₅₅₂ with a state-of-the-art 2P-compatible GEVI in awake mice (FIG. 11C-11F), although co-expression does not allow for ideal expression of either GEVI (FIG. 14A-14F). Distinct excitation wavelengths, acousto-optic modulator shuttering, and fluorescence emission bandpasses provided separation of the two GEVI signals (Methods and FIG. 13A-13D). Generally, good correlation was observed between the fluorescence signals of 2Photron-ST₅₅₂ and JEDI-2P-Kv, both for subthreshold and spike signals (FIG. 11C). A linear regression of the 2Photron-ST₅₅₂ ΔF/F versus that of JEDI-2P-Kv for simultaneous recordings from 13 cells across 4 mice showed that JEDI-2P-Kv was ˜3x more sensitive than 2Photron-ST₅₅₂ under these co-expression conditions (FIG. 11D). From a larger pool of 16 recorded neurons, including GCaMP co-expression, 2Photron-ST₅₅₂ showed a mean spike amplitude of 5.72±1.39% ΔF/F, 2× lower than previously reported for JEDI-2P-Kv (from Liu et al., 2022, n=34, Wilcoxon rank sum test: p=1.64e-8) (FIG. 11F).¹⁴ During spike depolarization and repolarization, 2Photron-ST₅₅₂ exhibited 2.1× and 3.4× faster kinetics of fluorescence change than JEDI-2P-Kv (FIG. 11E). Correspondingly, spikes with 2Photron-ST₅₅₂ were significantly narrower (FIG. 11E-11F) (2Photron-ST₅₅₂: FWHM of 1.42±0.13 ms [1.15-1.70 ms], JEDI-2P: 1.84±0.46 [1.25-3.55 ms] (Wilcoxon rank sum test: p=2.91e-05, n=16 cells, 5 mice). 2Photron-ST₅₅₂ showed brighter fluorescence output (15.5±4.9 MHz [9.68-24.20 MHz], with ˜4× higher photon flux compared to JEDI-2P-Kv (3.76±1.42 MHz [1.71-7.76 MHz], n=34, Wilcoxon rank sum test: p=1.64 e-08, FIG. 11F). Taken all together, the higher brightness of 2Photron-ST₅₅₂ partially offsets its lower sensitivity such that the signal-to-noise ratio (SNR) for spikes is within 50% of JEDI-2P and 40% greater than ASAP3 (FIG. 11F), thus enabling single action potential detection in the red channel with a 2P microscope (FIG. 11G).

### Example 4—Simultaneous GCaMP Calcium and 2Photron Voltage Recordings In Vivo

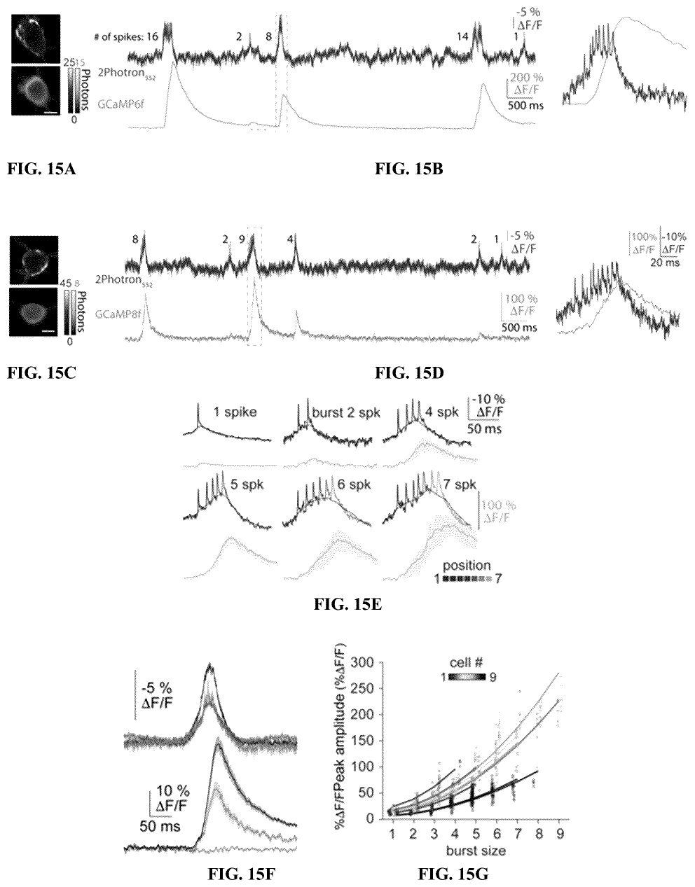

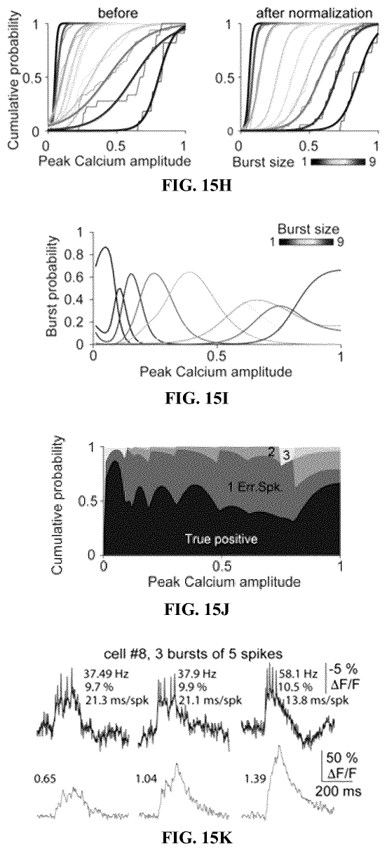

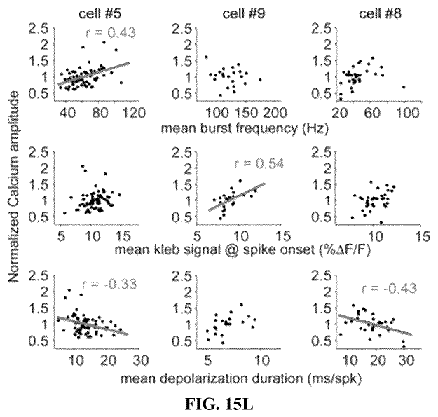

Cell-attached voltage recording together with genetically encoded calcium indicator (GECI) imaging in anesthetized rodents has shown that a given number of action potentials in a burst can generate fluorescence transients of highly variable amplitudes²³⁻²⁶. This variability cannot be explained by a unified biophysical model applying to all cells²⁷ and could partly be due to the perturbation of the recording electrode. Using AAVs, 2Photron-ST₅₅₂ was co-expressed with GCaMP6/8f (FIG. 15A-15E and FIG. 14F) to probe with minimal invasiveness the relationship between neuron electrical activity and cytoplasmic calcium transients in layer II/III of the neocortex of awake mice.

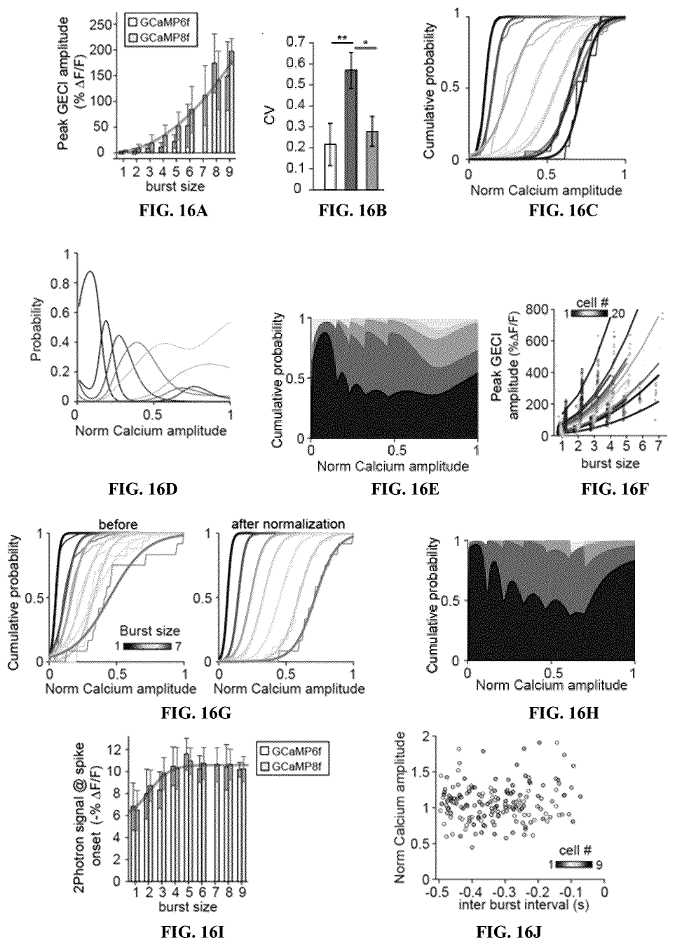

1686 bursts of spikes were extracted from simultaneous voltage and calcium recordings obtained from 20 cells in 6 mice. It was first verified that sub-threshold depolarizations without spikes did not evoke detectable GECI (GCaMP8f) fluorescence changes, while amplitude-matched depolarization with spikes did (FIG. 15F). As previously reported, a supralinear GCaMP ΔF/F increase as a function of burst size was observed, which was well fitted by a quadratic function (FIG. 16G and FIG. 16A). The range of GCaMP ΔF/F response amplitudes, however, displayed large cell-to-cell variability that could be quantified by the amplitude scaling factor of the quadratic fit of each cell (mean±std: 14.2±5.34, range: 6.9-24.4, n=9 cells). Normalizing GCaMP fluorescence amplitudes by this fitted scaling factor for each cell nearly abolished the intercellular source of variability; it reduced the average CV of the population amplitude distribution for a given spike number from 0.41±0.13 to 0.26±0.08 (FIG. 16B), which is similar to the average CV calculated from each individual cell (0.21±0.06, rank sum test, p=0.11).

The probability distribution of spike burst size for a given GCaMP response amplitude could then be calculated for the scaled data, yielding a best-case situation to test for burst size prediction (FIG. 4i). Because of the overlap between these probability distributions, prediction of spike burst size for a given GCaMP response amplitude has significant uncertainty. The percentage of GECI transients for which the most likely spike number matched the GEVI recording was 51.7% from raw data and improved to 60.5% after normalization (FIG. 15J). To provide a quantitative estimate of the spike assignment error, the absolute value of the difference between the most likely spike number and the actual spike number was computed. Normalization decreased the error from 0.517 spikes to 0.415 spikes per event. These results indicate that the best estimation, knowing the true distribution of GCaMP response amplitudes, would still lead to an approximate determination of spike number per transient. Using a realistic normalization for GECI-only recordings (FIG. 16C-16E), where each cell's transient amplitude is normalized by the highest amplitude seen for that cell, yielded an intermediate true spike number estimation of 55.1% and 0.591 average spike-number error per event. Replication of this analysis using a dataset from the literature²⁸, which combined in vivo juxtacellular electrophysiology and GCaMP8f calcium imaging, yielded similar results (FIG. 16F-16H), indicating that GCaMP response variability is not a consequence of voltage recording conditions.

GCaMP response amplitude variability for a given number of spikes might be explained both by the biophysics of calcium binding to the GECI²⁷ and by the time and voltage dependence of the various sources of calcium influx. The variability of GCaMP response around its mean (for the same cell and the same spike burst size) was examined to determine whether it correlated to any parameters of the voltage signal during the burst. The 2Photron-ST₅₅₂ signal preceding the last spike of the burst increased with burst size from 6.61 to 10.62% ΔF/F and then saturated (99th percentile after 5.01 spikes; FIG. 16I). Measurements were made of 1) the mean 2Photron-ST₅₅₂ signal at spike onset during the burst, 2) the mean intra-burst frequency, and 3) the mean duration of the depolarization plateau underlying each burst to see whether they could explain the GCaMP response variability. In 7 out of 9 cells, GCaMP response amplitude variation correlated significantly with one of these three parameters (Pearson coefficient range: −0.434 to 0.54, FIG. 15K, 15L) while none of the cells displayed significant correlation of the GCaMP response amplitude with the interval from the preceding burst (Pearson coefficient range: −0.222 to 0.2718, FIG. 16J). As these three parameters were either anti-correlated (10/27 pairs, Pearson coefficient range: −0.861 to −0.549) or correlated (9/27 pairs, Pearson coefficient range: 0.311 to 0.465) for the nine neurons recorded, partial correlation analysis was also performed. Notably, in 6/9 cells, GCaMP response amplitudes displayed a significant partial correlation with either the mean intra-burst frequency (2/6 cells R: −0.239 & 0.3824), the mean GEVI signal at spike onset (5/6 cells, R: −0.35 to 0.737) or the depolarization plateau underlying each burst (4/6 cells, R: −0.423 to 0.666). These data taken together suggest that the relationship between the GCaMP response amplitudes and electrophysiological parameters of a spike burst is multifactorial and cell-specific, making precise inference of spikes from the GCaMP ΔF/F signal difficult.

### Example 5—Reagent Availability

Plasmids have been deposited at Addgene (addgene.org) as follows: pCAG-2Photron-ST (plasmid #224359), pCAG-PaR-HaloTag-ST (plasmid #224360), pAAV-syn-FLEX-2Photron-ST (plasmid #224361).

### Example 6—Molecular Biology

The genes for all opsins described were synthesized (Integrated DNA Technologies) with mammalian codon optimization. HaloTag was amplified from the Voltron plasmid (addgene plasmid #119033). Rhodopsins and HaloTag were combined using overlap PCR. Cloning was done by restriction enzyme digest of plasmid backbones, PCR amplification of inserted genes, isothermal assembly, and followed by Sanger sequencing to verify DNA sequences. A soma localization tag (ST) was added using overlap PCR to make soma targeted versions of indicators. For expression in primary neuron cultures, sensors were cloned into a pcDNA3.1-CAG plasmid (Invitrogen). The amino acid sequences of all tested opsin-HaloTag fusions are given in FIG. 6, Table 1, and Table 2. For the preparation of viruses, plasmid DNA was purified by the Janelia Molecular Biology Facility and AAVs were prepared by the Janelia Viral Tools Team.

### Example 7—Imaging of Primary Neuron Cultures

Primary rat hippocampal neurons were prepared and transfected as described previously^{3, 30}. Stock solutions of JF525 HaloTag ligand was prepared at 1 mM in DMSO. Cultured neurons were incubated at 37° C. for 20-30 min at a final concentration ˜500 nM before washing twice with imaging buffer containing 145 mM NaCl, 2.5 mM KCl, 10 mM glucose, 10 mM HEPES, pH 7.4, 2 mM CaCl₂) and 1 mM MgCl₂. Synaptic blockers (10 μM CNQX, 10 μM CPP, 10 μM GABAZINE, and 1 mM MCPG) were added prior to imaging. Wide-field 1-photon imaging was performed on an inverted Nikon Eclipse Ti2 microscope equipped with a SPECTRA X light engine (Lumencore) with a 40× objective (NA=1.3, Nikon), and imaged onto a sCMOS camera (Hamamatsu ORCA-Flash 4.0). A custom filter set (510/25 nm (excitation, Lumencore), 545/40 nm (emission, Chroma), and a 525LP dichroic mirror (Chroma)) was used to image all opsin-HT fusions labeled with JF525 in culture.

### Example 8—Field Stimulation of Primary Neuron Cultures

A stimulus isolator (A385, World Precision Instruments) with platinum wires was used to deliver field stimuli (50V, 1 ms) to elicit action potentials in cultured neurons as described previously³⁰. The stimulation was controlled using Wavesurfer and timing was synchronized with fluorescence acquisition using Wavesurfer and a National Instruments PCIe-6353 board. To measure fluorescence response to action potentials over time, neurons were labeled with JF525 HaloTag ligand and imaged with a 40× objective at 400 Hz. Field electrode stimulations were applied to induce a train of 10 single action potentials at 66 Hz, followed by a train of 10 action potentials at 10 Hz. Images were processed in ImageJ. Voltage-dependent fluorescence changes were measured for hand-segmented neurons, on 2-3 well replicates and multiple fields of view per well.

### Example 9—Simultaneous Electrophysiology and Fluorescence Imaging in Primary Neuron Culture Under Both 1p and 2p Illumination

All imaging and electrophysiology measurements were performed in imaging buffer (145 mM NaCl, 2.5 mM KCl, 10 mM glucose, 10 mM HEPES, pH 7.4, 2 mM CaCl₂, 1 mM MgCl₂) adjusted to 310 mOsm with sucrose. For voltage clamp measurements, 500 nM TTX was added to the imaging buffer to block sodium channels. Synaptic blockers (10 μM CNQX, 10 μM CPP, 10 μM GABAZINE, and 1 mM MCPG) were added to block ionotropic glutamate, GABA, and metabotropic glutamate receptors³⁰.

Glass capillary with filament (Sutter Instruments) were pulled to a tip resistance of 4-6 MQ. Internal solution for current clamp recordings contained the following: 130 mM potassium methanesulfonate, 10 mM HEPES, 5 mM NaCl, 1 mM MgCl₂, 1 mM Mg-ATP, 0.4 mM Na-GTP, 14 mM Tris-phosphocreatine, adjusted to pH 7.3 with KOH, and adjusted to 300 mOsm with sucrose. Internal solution for voltage clamp recordings contained the following: 115 mM cesium methanesulfonate, 10 mM HEPES, 5 mM NaF, 10 mM EGTA, 15 mM CsCl, 3.5 mM Mg-ATP, 3 mM QX-314, adjusted to pH 7.3 with CsOH, and adjusted to 300 mOsm with sucrose.

Pipettes were positioned with a MP-285 manipulator (Sutter Instruments). Whole cell voltage clamp and current clamp recordings were acquired using an Axon700B amplifier, filtered at 10 kHz with the internal Bessel filter, and digitized using a National Instruments PCIe-6353 acquisition board at 20 kHz. Data were acquired from cells with access resistance <25 MQ. WaveSurfer software was used to generate the various analog and digital waveforms to control the amplifier, camera, light source, and record voltage and current traces. For fluorescence voltage curves, cells were held at a potential of −70 mV at the start of each step and then 0.5 s voltage steps were applied to step the potential from −145 mV to +55 mV in 25 mV increments. For current-clamp recordings to generate action potentials, current was injected (60-200 pA for 0.5 s) and voltage was monitored.

Five hundred thirty (530) nm LED light (Thorlabs) and Monaco 1035 (50 mW out of objective, repetition rate set to 10 MHZ) were used as 1p and 2p excitation light sources. For measurements in FIG. 7A-7E and FIG. 9A-9F, 2p laser was patterned into a stationary broadened focal spot by moving the lens in front of the objective into a defocused position. For measurement in FIG. 10A-10B, the galvos were programed to circularly scan the neurons at 1 KHz. 1p and 2p light were sequentially applied for 1 s. Cell were imaged using an upright Microscope, under 16× magnification (CFI75 LWD, NA 0.8, WD 3.0 mm, Nikon Instruments) objective with a filter set (525/50 nm for excitation, 562 nm dichroic mirror and 607/70 nm for emission). Fluorescence Images were collected with a scientific CMOS camera (Andor) and images acquisition were performed using Andor Solis software with a resolution of 128×128 pixels and a frame rate of either 500 or 1000 Hz. For FIG. 8, data were collected on an inverted Nikon Eclipse Ti2 microscope equipped with a SPECTRA X light engine (Lumencore) with a 40× objective (NA=1.3, Nikon) and a 550/15 nm excitation filter. A dichroic mirror (89100bs; Chroma) and 605/52 nm emission filters (Chroma) were also used. Image acquisition rate is 3.2 kHz. Fluorescence intensity was quantified using ImageJ software and data analysis performed in MATLAB and Prism GraphPad.

### Example 10—Simultaneous Dual Fluorescence Optical Recordings in Cortical Neurons in Behaving Mice

All protocols adhered to the guidelines of the French National Ethics Committee for Sciences and Health report on Ethical Principles for Animal Experimentation in agreement with the European Community Directive 86/609/EEC under agreement #29791.

### Example 11—Viral Vector Construction and Packaging

The 2Photron-Ts-Halotag construct is described above and the Voltron2-Ts-Halotag construct has been published³¹. All GEVIs were expressed as CRE-dependent type 1 AAVs under the control of the human synapsin (hSyn) promoter except for JEDI2P in the cortex where EF1a was used. Injected titers were 3e10¹² GC/ml in the visual cortex and 4e10¹² GC/mL in the cerebellum. The CRE-dependent GECIs GCaMP6f (Addgene, 100833-AAV1) and GCaMP8f (Addgene, 162379-AAV1) under the control of hSyn promoter were used at final titers of 1e10¹² and 8e10¹¹ GC/ml, respectively. To drive the cre recombinase in the visual cortex, AAV2/1-hSyn-Cre (Addgene, AV-1-PV2676) was injected at a final concentration of 1.5-2e109 GC/ml. In the cerebellum, to target glycinergic granular layer neurons, heterozygous GlyT2-Cre mice were used (H. U. Zeilhofer, University of Zurich).

### Example 12—Animal Handling, Viral Injections, and Surgeries

9 male GlyT2-Cre, 9 male and 1 female wild-type C57BL/6J mice were housed in standard conditions (12-hour light/dark cycles, light on at 7 a.m., with water and food ad libitum). A preoperative analgesic was used (buprenorphine, 0.1 mg/kg), and Ketamine-Xylazine were used as anesthetics. Viruses were combined in PBS, 300 n of which was injected at a flow rate of 75 nl/min into the visual cortex (V1 coordinates from bregma: anteroposterior −3/−3.5 mm, mediolateral −2.5/−3 mm, and dorsoventral −0.3 mm from brain surface, 10 mice) or into the lobule IV-V of the cerebellar cortex (coordinates from bregma: anteroposterior −6 mm, mediolateral ±0.4 mm, and dorsoventral-0.32 mm from brain surface, 9 mice), of adult (>P40) mice (body weight 24-30 g). A custom-designed aluminum head-plate was fixed on the skull with layers of dental cement (Metabond). A 5 mm diameter (visual cortex) or custom-designed laser cut (cerebellum) #1 coverslip was placed on top of the targeted area and secured with dental cement (tetric evoflow). Mice were allowed to recover for at least 15 days before recording sessions and housed 2-4 mice per cage. Behavioral habituation was adopted, involving progressive handling by the experimenter with gradual increases in head-fixed duration³². One or 2 days before experiments, JF552 was prepared and retro-orboritally injected as described previously³. Mice were handled before recording sessions to limit restraint-associated stress, and experiments were performed during the light cycle.

### Example 13—ULoVE Voltage Optical Recording and Experimental Design

3-4 hour recording sessions were performed while mice behaved spontaneously on top of an unconstrained running wheel in the dark³². Recordings were performed using a custom-designed dual scanner of acousto-optic deflector (AOD)-based random-access multiphoton microscope (Karthala System) based on a previously described design¹³. Excitation was provided by a femtosecond laser (InSight X3, Spectra Physics) mode-locked at 920 and 1045 nm with a repetition rate of 80 MHz for a green or red optogenetic reporter, respectively.

A water-immersion objective (CFO Apo25XC W1300, 1.1 NA, 2 mm working distance, Nikon) was used for excitation and epifluorescence light collection. Laser power was set to deliver 15 mW post-objective and pre-sample, then adjusted for mono-exponential loss through tissue with a length constant of 170 μm. The power was further doubled to account for the greater excitation volume using ULoVE compared with that used in standard 2P laser scanning microscopy. ULoVE excitation patterns were either two or three multiplexed patterns per cell (4 patterns in total: red+green) placed on the cell membrane for GEVI excitation (see FIG. 13C) and positioned on the cytoplasm for GECI excitation. To avoid crosstalk between channels during dual excitation recordings, transmission at the AOM was set to 0 on the 920 nm laser line for recording red signals and vice versa on the 1045 nm laser line for recording green signals. As added controls, patterns were positioned outside of the targeted neuron. The efficiency of this is presented in FIG. 13D. Using four patterns, temporal resolutions of 3751 Hz (visual cortex) and 3751-4761 Hz (3 to 4 patterns) (cerebellum) were obtained. Signals were passed through an IR blocking filter (TF1, Thorlabs), split into two channels using a 562 nm dichroic mirror (Semrock), and passed to two H12056P-40 photomultiplier tubes (Hamamatsu) used in photon counting mode. A 510/84 filtered green channel was used for JEDI-2P and a 607/70 filtered red channel was used for the rhodopsin-based GEVIs. Cells chosen for recording were manually selected based on brightness sufficient to yield significant signal-to-noise (see FIG. 14F). Recordings were stopped after 10 min and repeated across multiple cells and mice (details in the figure legends).

### Example 14—Photon Flux Analysis

In the dual GEVI recording experiments of 2Photron+JEDI2P an inverse relationship between baseline signals was observed, such that no one cell displayed both GEVIs at the “normal” bright level seen when either was expressed singly. Thus, to quantify the photon flux of a large number of cells and to identify cells that were co-expressing the two GEVIs, 2P stacks (4 mice) were acquired at a 1 us pixel dwell time, 5.5 pixels per micron, and 2 μm step size in z. Cells hand-selected within the stacks were then positioned in a 100±25 pixel-sized region and the z plane centered at the cell equator. Half of the highest photon counts inside this box, ±one plane, were summed and the flux was expressed in MHz. As mentioned, the cells showing the brightest baseline JEDI-2P expression had weak baseline 2Photron brightness, so for the recordings shown in FIG. 11C-11G, GEVI photon flux had to be compromised at the expense of the detectability FIG. 14D-14F.

### Example 15—Signal Analyses

Traces were generated by summing the collected photons per ULoVE volume and photobleaching was corrected by bi-exponential fitting. After removing low-frequency drift using a zero-phase distortion filter (highpass: 0.5 Hz), the traces were converted to % ΔF/F taking the mean signal as Fo. Up and down states were then assigned using a double Gaussian fit on the histogram distribution and the down state was subtracted. To compare the subthreshold between 2Photron and JEDI-2P (FIG. 11C-11D), traces were lowpass filtered at 25 Hz, and a linear regression between them was performed to extract the slope. For the calcium trace, a similar approach was performed but the baseline used to convert the trace to % ΔF/F was the mode of the calcium trace photon count. To compare photon flux and related discriminability metrics between ASAP3, JEDI-2P and 2Photron (FIG. 11F), the values had to be normalize to account for changes in applied power, pattern number per cell and per channel and pattern efficiency compared to the original single cell and non-multiplexed ASAP3 related data such as correcting factor is 3.12 for JEDI-2P and 2.0 for 2Photron.

### Example 16—Spikes Extraction and Waveform Analyses

Spikes were detected with a custom-designed algorithm. Three metrics that encompass different aspects of spike shapes and thresholds were combined, all with a z-score above 2.5 in at least two of the metrics, and 2 as a minimal level for the third metric. The first metric is the highpass filtered trace (second order Butterworth filter with the lower limit set at 40 Hz). The second metric is a cumulative probability transform of the signal using the erf function for a duration of 1.6 ms. The third metric is the cumulative product of the 2^{nd} to the 5^{th} scale of the coif1 discrete wavelet transform followed by a global realignment that enables energy retrieval in various frequency bands in one peak. After detection, all individual events were visually inspected in this wavelet domain, and any questionable detections were ruled out.

To perform the ground truth analysis (FIG. 14A-14F), the JEDI-2P detected spikes were taken as reference. Spikes falling within 3 ms of a given reference spike qualified as a true positive.

Sensitivity is defined as the ratio between the number of detected spikes and the number of reference spikes. Specificity is the ratio between false positives and the number of reference spikes. Precision is the ratio between the number of detected spikes and the number of all detected spikes. The error rate corresponds to 1 minus the harmonic mean of sensitivity and precision³³.

To compute spike waveform-related metrics, a spike average waveform based on the onset was computed where the amplitude at the onset was set to 0% ΔF/F. Amplitude corresponds to the peak value of the spike waveform average. FWHM corresponds to the extent in time at half of its amplitude after a 10 KHz interpolation of the spike average waveform. Tau of the depolarization and repolarization were extracted from mono and bi-exponential fits of the average spike waveform, respectively. For cerebellar cells, AHP corresponds to the trough magnitude in the average waveform, and dynamic range is the absolute sum of the AHP and spike magnitude. Spike SNR is the spike amplitude (in photons) divided by the shot noise, corresponding to the square root of the mean photon count of the trace. D′ is computed as previously reported^{13, 34}.

Bursts were extracted as the group of spikes that have an inter-spike interval (isi) below a limit defined for each neuron (mean±std: 25.8+/−9.69 ms). To define this limit, the isi distribution, with a 1 ms time bin and in log-log scale, was fitted by a Gaussian curve and the burst duration limit corresponds to the duration at which the ordinate of the fit is 0.2.

To extract the metric that called 2Photron signal at spike onset (FIG. 15H, 15J, 15K), the baseline drift from MLspike³³ is obtained, which provides a value of this drift at each spike onset.

To extract the depolarization with and without spikes (FIG. 15L), first, the traces of all bursts above 3 spikes is averaged. This template is used to set the parameters of the differential transform of the trace, which provides enhanced contrast of such average depolarizations. Performing the bidirectional differential of the signal during a 15 ms period of up-state and a baseline of 50 ms, separated by a lag of 30 ms, generated peaks when depolarizations occurred in both traces.

A detection z-score threshold of two was set, together with a prominence of one z-score, and sorted the detected events according to the presence or absence of depolarizations also bearing spikes. Events with spikes and calcium transients preceding the detected events were discarded.

### Example 17—Calcium Transient-Related Analyses

All bursts selected for subsequent analyses were kept if their preceding isi was larger than 50 ms and the traces were back to baseline level before the onset of the next burst. Calcium transients were extracted using as an onset the first spike of the burst. The amplitude of a calcium transient corresponds to the maximal value following the last spike of the burst, from a trace smoothed with a Gaussian kernel of 5 ms. GCaMP8f cells that strictly displayed more than 3 burst sizes were kept for subsequent analyses (9/12 cells).

To correct for inter-cell variance, the calcium transient amplitude was normalized at the single-cell level, either using realistic or scaled normalization. Realistic normalization tends to minimize differences between cells by normalizing amplitudes using the dynamic range (lowest-highest; aka 1 to 99 percentiles of the distribution) seen for that cell.

$$realistic normalizd amplitude ( cell ) = ( \frac{amplitues ( c ell ) - 1 th prctile ( cell )}{99 th prctile ( cell )} )$$

The scaled normalization is done in two steps, first, the mean of calcium transient amplitude per burst size is computed. Then a quadratic function is fitted using the following equation:

fit(cell)=αx²

Then the residual amplitude is minimized and the b parameter is adjusted for the whole cell population (b=4.108 and 2.333 respectively for the 2Photron and Zhang datasets).

Then cells are refitted to determine the scaling factor α:

$$fit ( cell ) = {α}_{c e l l} ( {x}^{2} + b )$$

and then this scaling factor is used for the normalization such as:

$$scaled normalizd amplitude ( cell ) = ( \frac{amplitudes ( ce l l )}{{α}_{cell}} )$$

To study calcium amplitude distributions as a function of burst size, the cumulative normalized calcium transient distributions (0.05 bin size) were fit using sigmoid functions for each burst size. The derivative of those fits is used to obtain the probability of event amplitudes knowing the burst size.

To compute true and false probabilities, the probability of obtaining an event of any burst size is computed knowing the amplitude using the following:

$$p ( burst \vert amp ) = \frac{p ( amp \vert burst ) * p ( burst )}{p ( amp )}$$

Where p(amp|burst) is the probability of event amplitudes knowing the burst size, p(burst) is the fraction of bursts occurring amongst all bursts included in the analyses, and p(amp) is the fraction of normalized amplitudes regardless of the burst size (obtained from the derivative of the sigmoid fits).

To find a true positive fraction, the true burst size was ascertained for a given amplitude range simply by taking the burst size that had the highest probability distribution. The true positive amplitude corresponds to the fraction of events for this tested amplitude for which the true burst size is equal to the tested burst size. If the tested burst size is equal to the true burst size±1 spike/burst it falls in the false positive 1 spike error category and similarly for 2, 3, and up to ±4 spikes/burst maximal error.

To provide a single value for true and errors over the range of tested amplitudes, the mean values were computed of those true and false positive fractions weighted by the distribution probabilities of amplitudes.

To evaluate whether voltage-related metrics can explain part of the variance of the calcium transient amplitudes, an analysis was performed at a single event level by selecting all isolated bursts having at least 2 spikes in the burst (n=650 bursts). The tested metrics were chosen not to be redundant and thus independent of the number of spikes per burst. Thus, the mean frequency corresponding to the average isi within the burst, the duration of the depolarization (the sigma of a Gaussian fit of the depolarization) divided by the burst size, and the mean depolarization amplitude taken at spike onset were selected. Calcium transient amplitudes were normalized per cell and burst size to only probe correlation to the inter-event variability. Linear correlations were performed per cell and Bonferroni correction was applied to account for multiple testing. Partial correlations per cell were performed to see if the identified significant pairwise correlations resist the high interdependencies that exist between voltage-related metrics.

### Example 18—Discussion of Studies Described in Examples

Optically recording voltage using GEVIs has remained challenging, largely due to the demands imposed by rapid acquisition of fast signals like action potentials. Recent high-speed 2P microscopy approaches have taken advantage of the high power output, good stability, and low cost of fixed wavelength lasers that output in the range of 1030-1080 nm^{17,18}. These wavelengths, however, do not efficiently excite GFP-based probes²⁹. However, as disclosed herein, a chemigenetic approach was used to combine a unique rhodopsin voltage sensor domain with fluorescence output from bright synthetic dyes that can be efficiently excited beyond 1030 nm and have a red-shifted emission that is separable from GFP for multiplexed imaging.

Although past efforts to image rhodopsin-based GEVIs with 2P largely failed, there have been some recent developments as described herein. Cumulatively, these efforts shed significant light on the problem. A common conclusion is that FRET rhodopsin GEVIs can indeed be used with 2P illumination, but the details of the illumination conditions can influence the amplitude and kinetics of fluorescence change¹¹. It remains unclear from which intermediates (or ground state) of the photocycle the voltage sensitivity of rhodopsin arises. Different illumination schemes (e.g. wavelength, frequency and intensity etc.) might drive the rhodopsin into different photointermediate population distributions, potentially affecting its voltage sensitivity. As disclosed herein, parallel, continuous 2P illumination performed well, and circular point scanning also worked for in vitro applications (Table 3). ULoVE excitation from 80 MHz laser pulses allowed good quality in vivo optical voltage recordings from 2Photron (Table 3).

TABLE 3
Summary of voltage optical recording conditions

| Illumination | Microscope | Light source | Light power |
|---|---|---|---|
| 1-Photon | 1P widefield | LEDs (Spectra X light engine, | 10-50 mW/mm² |
| | (Nikon Ti2) | Lumencore) | |
| 2-Photon | 2P SLAP | 1030 nm laser (Yb:YAG, | 40 mW out of |
| | microscope | 1,030 nm, 190 fs, tunable | objective |
| | | repetition rate 1-10 MHz; | |
| | | BlueCut, Menlo Systems) | |
| 1-Photon | 1P widefield | 530 nm LED light (Thorlabs) | 10-50 mW/mm² |
| 2-Photon | Laser circularly | Monaco 1035 nm laser, | 25.7 mW out of |
| | scanned the | repetition rate set to 10 MHz | objective |
| | neurons at 1 KHz | | |
| 2-Photon | Laser patterned | Monaco 1035 nm laser, | 50 mW out of |
| | into a stationary | repetition rate set to 10 MHz | objective |
| | broadened focal | | |
| | spot | | |
| 2-Photon | 2P AOD | InSight X3, Spectra Physics | 15 mW out of |
| | microscope | mode-locked at 1045 nm with a | objective |
| | | repetition rate of 80 MHz | |

As disclosed herein, unique rhodopsin GEVIs, 2Photron, were engineered and in vivo 2P voltage recording was demonstrated with single-trial, single-spike resolution over many minutes, compatible with a variety of behavioral paradigms in mice. Using a red-shifted dye with 2Photron frees the commonly used green fluorescence channel for simultaneous multiplexed monitoring of GFP-based indicators, and allowed for performing simultaneous recordings of voltage and calcium using 2Photron and GCaMP. The results highlighted the difficulty of precisely assigning spike numbers and timing using only calcium imaging, due to cell-to-cell variability in the voltage to calcium relationship that could not be accounted for using only GCaMP data. Voltage imaging will likely play an increasingly important role in reading out action potentials from circuits in vivo. Multiplexed 2P imaging will enable further dissection of circuit function through correlation of voltage signals with neurotransmitters, neuromodulators, or other aspects of cell signaling.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Wong-Campos, J. D. et al. Voltage dynamics of dendritic integration and back-propagation in vivo. bioRxiv 2023.05.25.542363 (2023) doi:10.1101/2023.05.25.542363.
2. Park, P. et al. Dendritic excitations govern back-propagation via a spike-rate accelerometer. bioRxiv 2023.06.02.543490 (2024) doi:10.1101/2023.06.02.543490.
3. Abdelfattah, A. S. et al. Bright and photostable chemigenetic indicators for extended in vivo voltage imaging. Science 365, 699-704 (2019).
4. Tian, H. et al. Video-based pooled screening yields improved far-red genetically encoded voltage indicators. Nature Methods 2023 20:7 20, 1082-1094 (2023).
5. Piatkevich, K. D. et al. Population imaging of neural activity in awake behaving mice. Nature 2019 574:7778 574, 413-417 (2019).
6. Kannan, M. et al. Dual-polarity voltage imaging of the concurrent dynamics of multiple neuron types. Science (1979) 378, (2022).
7. Evans, S. W. et al. A positively tuned voltage indicator for extended electrical recordings in the brain. Nature Methods 2023 20:7 20, 1104-1113 (2023).
8. Adam, Y. et al. Voltage imaging and optogenetics reveal behaviour-dependent changes in hippocampal dynamics. Nature 569, 413-417 (2019).
9. Chamberland, S. et al. Fast two-photon imaging of subcellular voltage dynamics in neuronal tissue with genetically encoded indicators. Elife 6, (2017).
10. Brinks, D., Klein, A. J. & Cohen, A. E. Two-Photon Lifetime Imaging of Voltage Indicating Proteins as a Probe of Absolute Membrane Voltage. Biophys J 109, 914-921 (2015).
11. Grimm, C. et al. Two-photon voltage imaging with rhodopsin-based sensors. bioRxiv 2024.05.10.593541 (2024) doi:10.1101/2024.05.10.593541.
12. Brooks III, F. P., Davis, H. C., Park, P., Qi, Y. & Cohen, A. E. Photophysics-informed two-photon voltage imaging using FRET-opsin voltage indicators. bioRxiv 2024.04.01.587540 (2024) doi:10.1101/2024.04.01.587540.
13. Villette, V. et al. Ultrafast Two-Photon Imaging of a High-Gain Voltage Indicator in Awake Behaving Mice. Cell 179, 1590-1608.e23 (2019).
14. Liu, Z. et al. Sustained deep-tissue voltage recording using a fast indicator evolved for two-photon microscopy. Cell 185, 3408-3425.e29 (2022).
15. Abdelfattah, A. S. et al. A Bright and Fast Red Fluorescent Protein Voltage Indicator That Reports Neuronal Activity in Organotypic Brain Slices. J Neurosci 36, 2458-2472 (2016).
16. Kannan, M. et al. Fast, in vivo voltage imaging using a red fluorescent indicator. Nat Methods 15, 1108-1116 (2018).
17. Wu, J. et al. Kilohertz two-photon fluorescence microscopy imaging of neural activity in vivo. Nature Methods 2020 17:3 17, 287-290 (2020).
18. Kazemipour, A. et al. Kilohertz frame-rate two-photon tomography. Nature Methods 2019 16:8 16, 778-786 (2019).
19. Kralj, J. M., Hochbaum, D. R., Douglass, A. D. & Cohen, A. E. Electrical spiking in Escherichia coli probed with a fluorescent voltage-indicating protein. Science (1979) 333, 345-348 (2011).
20. Kralj, J. M., Douglass, A. D., Hochbaum, D. R., MacLaurin, D. & Cohen, A. E. Optical recording of action potentials in mammalian neurons using a microbial rhodopsin. Nature Methods 2011 9:19, 90-95 (2011).
21. Abdelfattah, A. S. et al. A general approach to engineer positive-going eFRET voltage indicators. Nat Commun 11, (2020).
22. Sineshchekov, O. A., Govorunova, E. G., Li, H. & Spudich, J. L. Bacteriorhodopsin-like channelrhodopsins: Alternative mechanism for control of cation conductance. Proceedings of the National Academy of Sciences 114, E9512-E9519 (2017).
23. Theis, L. et al. Benchmarking Spike Rate Inference in Population Calcium Imaging. Neuron 90, 471-482 (2016).
24. Dana, H. et al. Sensitive red protein calcium indicators for imaging neural activity. Elife 5, (2016).
25. Huang, L. et al. Relationship between simultaneously recorded spiking activity and fluorescence signal in GCaMP6 transgenic mice. Elife 10, (2021).
26. Chen, T. W. et al. Ultrasensitive fluorescent proteins for imaging neuronal activity. Nature 499, 295-300 (2013).
27. Greenberg, D. S. et al. Accurate action potential inference from a calcium sensor protein through biophysical modeling. bioRxiv 479055 (2018) doi:10.1101/479055.
28. Zhang, Y. et al. Fast and sensitive GCaMP calcium indicators for imaging neural populations. Nature 615, 884-891 (2023).
29. Mohr, M. A. et al. jYCaMP: an optimized calcium indicator for two-photon imaging at fiber laser wavelengths. Nature Methods 2020 17:7 17, 694-697 (2020).
30. Wardill, T. J. et al. A Neuron-Based Screening Platform for Optimizing Genetically-Encoded Calcium Indicators. PLOS One 8, 1-12 (2013).
31. Abdelfattah, A. S. et al. Sensitivity optimization of a rhodopsin-based fluorescent voltage indicator. Neuron 111, 1547-1563.e9 (2023).
32. Villette, V., Levesque, M., Miled, A., Gosselin, B. & Topolnik, L. Simple platform for chronic imaging of hippocampal activity during spontaneous behaviour in an awake mouse. Sci. Rep. 7, (2017).
33. Deneux, T. et al. Accurate spike estimation from noisy calcium signals for ultrafast three-dimensional imaging of large neuronal populations in vivo. Nat. Commun. 7, (2016).
34. Wilt, B. A., Fitzgerald, J. E. & Schnitzer, M. J. Photon shot noise limits on optical detection of neuronal spikes and estimation of spike timing. Biophys. J. 104, 51-62 (2013).
35. Hochbaum D. R. et al., All-optical electrophysiology in mammalian neurons using engineered microbial rhodopsins. Nat. Methods. 11, 825-833 (2014).
36. U.S. Pat. No. 11,385,236 to Schreiter, et al. for CHEMIGENETIC VOLTAGE INDICATORS.
37. U.S. Pat. No. 11,598,792 to Schreiter, et al. for VOLTAGE INDICATORS.

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A voltage indicator, comprising:
a voltage-sensitive rhodopsin domain comprising an amino acid sequence selected from the group consisting of SEQ ID NO: 1-26, including a mutation to replace a charged canonical amino acid residue with a neutral residue at one, two, or three residues within a pump or channel core motif of the rhodopsin domain; and
a capture protein that covalently or noncovalently binds a fluorescent element that is a fluorescent protein, or a fluorescent dye;
wherein the capture protein is provided together with the voltage-sensitive rhodopsin domain in a fusion protein.

2. The voltage indicator of claim 1, wherein the rhodopsin domain comprises an amino acid sequence selected from the group consisting of SEQ ID NO: 31-74.

3. The voltage indicator of claim 1, wherein the rhodopsin domain comprises the amino acid sequence of SEQ ID NO: 1, including one or two mutations to replace charged canonical amino acid residues at counterion positions with neutral residues.

4. The voltage indicator of claim 3, wherein the one or two mutations are selected from the group consisting of: D89X, D100X, and E206X, where X is a neutral residue.

5. The voltage indicator of claim 1, wherein the rhodopsin domain comprises the amino acid sequence of SEQ ID NO: 31.

6. The voltage indicator of claim 1, wherein the rhodopsin domain comprises the amino acid sequence of SEQ ID NO: 2, including one or two mutations to replace charged canonical amino acid residues at counterion positions with neutral residues.

7. The voltage indicator of claim 6, wherein the mutation is selected from the group consisting of: D136X, D147X, and E254X, where X is a neutral residue.

8. The voltage indicator of claim 1, wherein the rhodopsin domain comprises the amino acid sequence of SEQ ID NO: 32.

9. The voltage indicator of claim 1, wherein the capture protein is selected from the group consisting of a self-labeling protein and avidin.

10. The voltage indicator of claim 1, wherein the capture protein is a self-labeling protein.

11. The voltage indicator of claim 10, wherein the fluorescent element is the fluorescent dye, and the self-labeling protein binds the fluorescent dye via a self-labeling protein ligand.

12. The voltage indicator of claim 1, further comprising a 1, 2, 3, 4, 5, 6, 7, 8, 9, or 10 amino acid linker between the voltage-sensitive rhodopsin domain and the capture protein.

13. The voltage indicator of claim 1, and further comprising a fluorescent element.

14. The voltage indicator of claim 1, further comprising a targeting sequence and/or an endoplasmic reticulum (ER) export sequence.

15. A nucleotide molecule, comprising a nucleotide encoding an amino acid sequence selected from the group consisting of SEQ ID NO: 1-26, including a mutation to replace a charged canonical amino acid residue with a neutral residue at a residue within a pump or channel core motif of the rhodopsin domain.

16. The nucleotide molecule of claim 15, comprising a nucleotide encoding an amino acid selected from the group consisting of SEQ ID NO: 31-74.

17. The nucleotide molecule of claim 15, and further comprising a nucleotide encoding an amino acid sequence for a capture protein.

18. A vector, comprising the nucleotide of claim 17.

19. A method of measuring voltage, the method comprising:
(i) delivering to a cell a nucleotide encoding an amino acid molecule comprising
a voltage-sensitive rhodopsin domain comprising an amino acid sequence selected from the group consisting of SEQ ID NO: 1-26, including a mutation to replace a charged canonical amino acid residue with a neutral residue at a residue within a pump or channel core motif of the rhodopsin domain; and
a capture protein that covalently or noncovalently binds a fluorescent element that is a fluorescent protein, or a fluorescent dye;
wherein the capture protein is provided together with the voltage-sensitive rhodopsin domain in a fusion protein; and
(ii) delivering to the cell a fluorescent element that is a fluorescent protein or a fluorescent dye, wherein
when the fluorescent element is a fluorescent protein, it is provided together with the capture protein and the voltage-sensitive rhodopsin domain in a fusion protein; and
when the fluorescent element is a fluorescent dye, it is attached to a ligand of the capture protein; and
(iii) determining changes in fluorescence of the fluorescent element, as an measurement of voltage changes in the cell, wherein an increase in membrane potential lead to an increase in fluorescence.

20. The method of claim 19, wherein the microscope is a two-photon microscope.


SEQUENCE LISTING (135 sequences)

SEQ ID NO: 1 - protein, 236 aa, Klebsormidium nitens
MVYSHADAAGWTLYWITYGIMAVTALIFFAMSLRRPIQQRSHHYTSFLIVAIASLAYYAMASQGGNTRIRVYQGPADSYRQIFWARYVDWFFTTPLLLLDLVLLSNLSKLRIAAIMVADIFMILTGLFGAVEARSNKWGWFVFGCIFMLYIFYELLVNVRKGAYARGGQHGMLYSVLLVWLLILWVQYPVVWGLAEGSSTVSSDTEIAWYAALDICAKCVFGFILLLGIESIDRKR

SEQ ID NO: 2 - protein, 284 aa, Podospora anserine
MIHHDQVAEMLYGYAGAAAAKPTHTDSPGPIPTVIPTPPQFQEIGETGHRTLWVVFALMVLSSGFFAFMSWNVPISKRLYHVITTLITITASLSYFAMASGHVTSFSCTPAKDHHKHVPDVGYTECRQVFWGRYVDWAITTPLLLLDLSLLAGIDGAHTLMAVIADVIMVLSGLFASQGETATQRWGWYAIGCVSYLFVIWHVALHGARTVTAKGRGVTRLFSSLALFTFVLWTAYPIVWGIADGAHRTTVDTEILIYAVLDILAKPVFGLWLLFSHRSLAETN

SEQ ID NO: 3 - protein, 238 aa, Haloarcula argentinensis
MPEPGSEAIWLWLGTAGMFLGMLYFIARGWGETDSRRQKFYIATILITAIAFVNYLAMALGFGLTIVEFAGEEHPIYWARYSDWLFTTPLLLYDLGLLAGADRNTITSLVSLDVLMIGTGLVATLSPGSGVLSAGAERLVWWGISTAFLLVLLYFLFSSLSGRVADLPSDTRSTFKTLRNLVTVVWLVYPVWWLIGTEGIGLVGIGIETAGFMVIDLTAKVGFGIILLRSHGVLDGAA

SEQ ID NO: 4 - protein, 288 aa, Leptosphaeria maculans
MIVDQFEEVLMKTSQLFPLPTATQSAQPTHVAPVPTVLPDTPIYETVGDSGSKTLWVVFVLMLIASAAFTALSWKIPVNRRLYHVITTIITLTAALSYFAMATGHGVALNKIVIRTQHDHVPDTYETVYRQVYYARYIDWAITTPLLLLDLGLLAGMSGAHIFMAIVADLIMVLTGLFAAFGSEGTPQKWGWYTIACIAYIFVVWHLVLNGGANARVKGEKLRSFFVAIGAYTLILWTAYPIVWGLADGARKIGVDGEIIAYAVLDVLAKGVFGAWLLVTHANLRESD

SEQ ID NO: 5 - protein, 281 aa, Neurospora crassa
MIHPEQVADMLRPTTSTTSSHVPGPVPTVVPTPTEYQTLGETGHRTLWVTFALMVLSSGIFALLSWNVPTSKRLFHVITTLITVVASLSYFAMATGHATTFNCDTAWDHHKHVPDTSHQVCRQVFWGRYVDWALTTPLLLLELCLLAGVDGAHTLMAIVADVIMVLCGLFAALGEGGNTAQKWGWYTIGCFSYLFVIWHVALHGSRTVTAKGRGVSRLFTGLAVFALLLWTAYPIIWGIAGGARRTNVDTEILIYTVLDLLAKPVFGFWLLLSHRAMPETN

SEQ ID NO: 6 - protein, 235 aa, Acetabularia acetabulum
MSNPNPFQTTLGTDAQWVVFAVMALAAIVFSIAVQFRPLPLRLTYYVNIAICTIAATAYYAMAVNGGDNKPTAGTGADERQVIYARYIDWVFTTPLLLLDLVLLTNMPATMIAWIMGADIAMIAFGIIGAFTVGSYKWFYFVVGCIMLAVLAWGMINPIFKEELQKHKEYTGAYTTLLIYLIVLWVIYPIVWGLGAGGHIIGVDVEIIAMGVLDLLAKPLYAIGVLITVEVVYGK

SEQ ID NO: 7 - protein, 233 aa, Chlorella vulgaris
MAVHQIGEGGLVMYWVTFGLMAFSALAFAVMTFTRPLNKRSHGYITLAIVTIAAIAYYAMAASGGKALVSNPDGNLRDIYYARYIDWFFTTPLLLLDIILLTGIPIGVTLWIVLADVAMIMLGLFGALSTNSYRWGYYGVSCAFFFVVLWGLFFPGAKGARARGGQVPGLYFGLAGYLALLWFGYPIVWGLAEGSDYISVTAEAASYAGLDIAAKVVFGWAVMLSHPLIARNQ

SEQ ID NO: 8 - protein, 258 aa, Taphrina deformans
MSSLFEKRNTAVATNYRGPTNSIVINEAGSDWYWAVFSVMAASAIVFSVMAAMTPRGERVFHYLTIAIVSVASVAYFTMAADLGSVAIISEFANYSSLPTRQVFYARYIDWVITTPLLLTDLMLLAGLPWSTIIFTIVMDEVMVLTGLFGAITPSSYKWGYFTFGMVAYFFVAWVLIVEARKNAHRLGSDVHRLYIGIAIWTATLWTLYPVAWGLSEGGNVTSSDGEAIFYGVLDLLAKPVFGLWILLGHKGIGMDRL

SEQ ID NO: 9 - protein, 263 aa, Exophiala sideris
MESNLVRRNGALTVNTMTQNNQSVAIHQTVRGSDWYYTVCAVMGTTSLAILALSRMKPRTDRVFFYLTSGLCMVACIAYFAMGSNLGWTPIDVEWLRNDSVVRGVNRQVFYARYIDWVITTPMLLMDLLLTAGMPWPTILWIILLDEIMIVTGLIGALVKSRYKWGFYVFGCMAMFYIMWELAFPARKHAKVLGKDIHRSFVLCGVLTLVVWLCYPICWGLSEGGNVISPDSESVFYGVLDVLAKPSFSIALIATHWNIDPGR

SEQ ID NO: 10 - protein, 257 aa, Moelleriella libera
MGNSAIEVNGDTVDSYTADVKITTHGSDAYWAITAVMAFTTILFIAHSFTKPRTDRIFHYITISITLVASIAYFTMASNLGWASIFIEFQRDDPLVSGTTREIFYVRYIDWVITTPLLLLDILLTAGLPWPTILFTILLDEVMIITGLVGALVKSSYKWGFFTFGCVAFFGVAWSVAWTGRKHANALGSDIGRVYLMTSVWTLFLWLLYPIAWGVSEGGNVISPDSEAAFYGTLDVLAKPIFGIILLWGHRNIPASR

SEQ ID NO: 11 - protein, 252 aa, Saitoella complicate
MDSLLLQRRNDAVATNPPNATFALTENGSSWLWAVFCVMALSCIIIAALSLFKPMGYRIFYLLNVAILATASVSYFSLASDLGLTPVTVEFRGPGTRQIAYVRYIDWFVTTPLLLTELLLTAGLPTNIIISTIFADLVMIITGLAGALVVSRYKWGYYTMGCVAMLWVFWNVFTGIKVSGNIGPDVRKSYTLLAVWLMIIWLNYPICWGLAEGGNRITVVGEMVYYGVLDLLAKPVFAAISLAVHSKIELSR

SEQ ID NO: 12 - protein, 267 aa, Rhodotorula graminis
MDAILSKRNEVLSLNPLVANIDITTAASDWLWAVFAVMGLSAIILLVLGHATRPIGERAFHELAAALCFTASIAYYSMASDLGATPIEVEFIRGGTLGQNWVDIGVLRPTRSIWYARYIDWTITTPLLLLELALTTALPLSQIFGLVFFDIVMIITGLLGALTASRYKWGFFVFGCVAMFWIFWVLFFPARKSASHLGTDYHRAYTSSAIVLCTLWTVYPIIWGVCDGGNVITPTSEMVAYGVLDLLAKPVFSFWHVFQLSRLDYAR

SEQ ID NO: 13 - protein, 256 aa, Rhodotorula toruloides
MPFYIKNADIDITTHGSDWLWAVFSVMLLSAIGILVWGHVARPLGERAFHELAAALCFTASIAYFAMASDLGDVPIVVEFIRGGSLGQNWVQVGVENPTRAIWYARYIDWTITTPMLLLELLLCTGLPLSQVFSVIFADLLMIETGLIGALVASRYKWGFYAFGCAAQLYIWWMLLVPGRRSAQHIGSDFAKSYTMSNIFLTTVWLVYPVIWGVADGGNVITPDSEMIAYGVLDLLAKPVFSVIHLMSLSKLDYAR

SEQ ID NO: 14 - protein, 242 aa, Krokinobacter eikastus
MKFLLLLLADPTKLDPSDYVGFTFFVGAMAMMAASAFFFLSLNQFNKKWRTSVLVSGLITFIAAVHYWYMRDYWFAIQESPTFFRYVDWVLTVPLMCVEFYLILKVAGAKPALMWKLILFSVIMLVTGYFGEAVFQDQAALWGAISGAAYFYIVYEIWLGSAKKLAVAAGGDILKAHKILCWFVLVGWAIYPLGYMLGTDGWYTSILGKGSVDVAYNIADAINKIGFGLVIYALAVKKNEVD

SEQ ID NO: 15 - protein, 273 aa, Krokinobacter eikastus
MTQELGNANFENFIGATEGFSEIAYQFTSHILTLGYAVMLAGLLYFILTIKNVDKKFQMSNILSAVVMVSAFLLLYAQAQNWTSSFTFNEEVGRYFLDPSGDLFNNGYRYLNWLIDVPMLLFQILFVVSLTTSKFSSVRNQFWFSGAMMIITGYIGQFYEVSNLTAFLVWGAISSAFFFHILWVMKKVINEGKEGISPAGQKILSNIWILFLISWTLYPGAYLMPYLTGVDGFLYSEDGVMARQLVYTIADVSSKVIYGVLLGNLAITLSKNK

SEQ ID NO: 16 - protein, 275 aa, Gloeobacter violaceus
MLMTVFSSAPELALLGSTFAQVDPSNLSVSDSLTYGQFNLVYNAFSFAIAAMFASALFFFSAQALVGQRYRLALLVSAIVVSIAGYHYFRIFNSWDAAYVLENGVYSLTSEKFNDAYRYVDWLLTVPLLLVETVAVLTLPAKEARPLLIKLTVASVLMIATGYPGEISDDITTRIIWGTVSTIPFAYILYVLWVELSRSLVRQPAAVQTLVRNMRWLLLLSWGVYPIAYLLPMLGVSGTSAAVGVQVGYTIADVLAKPVFGLLVFAIALVKTKAD

SEQ ID NO: 17 - protein, 258 aa, Salinibacter ruber
MLQELPTLTPGQYSLVFNMFSFTVATMTASFVFFVLARNNVAPKYRISMMVSALVVFIAGYHYFRITSSWEAAYALQNGMYQPTGELFNDAYRYVDWLLTVPLLTVELVLVMGLPKNERGPLAAKLGFLAALMIVLGYPGEVSENAALFGTRGLWGFLSTIPFVWILYILFTQLGDTIQRQSSRVSTLLGNARLLLLATWGFYPIAYMIPMAFPEAFPSNTPGTIVALQVGYTIADVLAKAGYGVLIYNIAKAKSEEE

SEQ ID NO: 18 - protein, 248 aa, Gammaproteobacteria
MKLLLILGSVIALPTFAAGGGDLDASDYTGVSFWLVTAALLASTVFFFVERDRVSAKWKTSLTVSGLVTGIAFWHYMYMRGVWIETGDSPTVFRYIDWLLTVPLLICEFYLILAAATNVAGSLFKKLLVGSLVMLVFGYMGEAGIMAAWPAFIIGCLAWVYMIYELWAGEGKSACNTASPAVQSAYNTMMYIIIFGWAIYPVGYFTGYLMGDGGSALNLNLIYNLADFVNKILFGLIIWNVAVKESSN

SEQ ID NO: 19 - protein, 250 aa, Gammaproteobacteria
MGKLLLILGSAIALPSFAAAGGDLDISDTVGVSFWLVTAGMLAATVFFFVERDQVSAKWKTSLTVSGLITGIAFWHYLYMRGVWIDTGDTPTVFRYIDWLLTVPLQVVEFYLILAACTSVAASLFKKLLAGSLVMLGAGFAGEAGLAPVLPAFIIGMAGWLYMIYELYMGEGKAAVSTASPAVNSAYNAMMMIIVVGWAIYPAGYAAGYLMGGEGVYASNLNLIYNLADFVNKILFGLIIWNVAVKESSN

SEQ ID NO: 20 - protein, 267 aa, Proteomonas sulcate
MTMLEHLEGTMDGWYAENDLGQGAIIAHWVTFFFHMITTFYLGYVSFHSKGPGGKQPYFAGYHEENNIGIFVNLFAAISYFGKVVSDTHGHNYQNVGPFIIGLGNYRYADYMLTCPLLVMDLLFQLRAPYKITCAMLIFAVLMIGAVTNFYPGDDMKGPAVAWFCFGCFWYLIAYIFMAHIVSKQYGRLDYLAHGTKAEGALFSLKLAIITFFAIWVAFPLVWLLSVGTGVLSNEAAEICHCICDVVAKSVYGFALANFREQYDREL

SEQ ID NO: 21 - protein, 241 aa, Guillardia theta
MVESSAVIAANWISFLVIAGSFVVLCFISLRYKGPGGNENYYNGFREQNMLTVIINLWCALAYFAKVLQSHSDDDGFVPLTKIPYLDYATTCPLLTLDLMWCLDAPYKITSAVLVFTVMITGVACSLAVAPYSFYWFAMGMVLFIFTYVLMLSIVRERLEFITQCAHDSNAKRSIKHLKAAVIIYFGIWPIFAILWLLSYRAANVISNDTNHILHCILDVIAKSCFGFVLLHFKMYFDKKL

SEQ ID NO: 22 - protein, 241 aa, Guillardia theta
MVASSAVITANWISFLAISASFIILLVISLRYKGPGGTESFYNGFKEQNMLTVFINLWCALAYFAKVLQSHSNDNGFAPLTVIPYVDYCTTCPLLTLDLLWCLDAPYKISSAVLVFTCLVIAVACSLAVAPFSYCWFAMGMVLFTFTYVFILSIVRQRLDFFTLCARDSNAKQSLKHLKTAVFIYFGIWLLFPLLWLLSYRAANVISNDINHIFHCILDVIAKSVYGFALLYFKMYFDKKL

SEQ ID NO: 23 - protein, 247 aa, Guillardia theta
MVSALDQNGPQYLQNPIVIAADWIGFIALFGSSLAVAYKLVTFKGPDQDDVYFFGYREEKMISVFVNLFAALAYWAKLASHANGDVGPAASVTTYKYLDYLFTCPLLTIDLLWCLNLPYKFTFGAIVAVCILCAFMASVIPPPARYMWFGMGITVFSAAWFNILKLVRMRLEQFVSKEAKKVRQSLKVACMTYFFIWLGYPTLWVLGDAGVLDSVVSALLHTFLDVFSKSIYGFALLHFVMRTDKRE

SEQ ID NO: 24 - protein, 260 aa, Guillardia theta
MTTSAPSLSDPNWQYGMGGWNNPRLPNFNLHDPTVIGVDWLGFLCLLGASLALMYKLMSFKGPDGDQEFFVGYREEKCLSIYVNLIAAITYWGRICAHFNNDMGLSLSVNYFKYLDYIFTCPILTLDLLWSLNLPYKITYSLFVGLTIACNAFEPPARYLWFMFGCFIFAFTWISIIRLVYARFQQFLNEDAKKIRAPLKLSLTLYFSIWCGYPALWLLTEFGAISQLAAHVMTVIMDVAAKSVYGFALLKFQLGVDKRD

SEQ ID NO: 25 - protein, 261 aa, Guillardia theta
MSTSSVAYLRTPVVQALDWVGFISLGGTAAVLAYRLMNFKPPNKDILYFFGYREKGMISLYVNLFAAVAYYARITSHLSGDVGAATNIILYKYFDYLITCPLLTFDLLTTLNLPYKITYAVYVQITIFTGFMSANTPPPATFLWFAFGMLLFSYTWFNIISLVQVRFIQYFAKKGNTTQSRRVSVASKAGFRNKNVRNPLQTALSTYFCIWMVYPVLWLLLKTKVIDQVTEHCINVVMDVLAKSMYGFALLRFQLLMDKAN

SEQ ID NO: 26 - protein, 255 aa, Actinomycetes bacterium
MAKPTVKEIKSLQNFNRIAGVFHLLQMLAVLALANDFALPMTGTYLNGPPGTTFSAPVVILETPVGLAVALFLGLSALFHFIVSSGNFFKRYSASLMKNQNIFRWVEYSLSSSVMIVLIAQICGIADIVALLAIFGVNASMILFGWLQEKYTQPKDGDLLPFWFGCIAGIVPWIGLLIYVIAPGSTSDVAVPGFVYGIIISLFLFFNSFALVQYLQYKGKGKWSNYLRGERAYIVLSLVAKSALAWQIFSGTLIP

SEQ ID NO: 27 - protein, 247 aa, Emiliania huxleyi
MDTIVLTINWFNFIAGLFHVALAIVCALLGDVSQTFKMYMPFTTFRNGTQENEVTIKYSGYFPMTVIFVVYFSVTALFHMGNAFLWNDTYHRFLSNRKNPIRWTEYSITAPLMTAILAFIAGSRNWLFVIAASTLTFASISVGFVLDEIRKYSFFVLGMIPFLVEFSLIILSLYLTTSCYPSYLPITIFVEFGLWILFPFVSLLELSGINYKIGELVFIVLSFVSKSVLAIILNSENVLKNGVTGIC

SEQ ID NO: 28 - protein, 269 aa, Heimdallarchaeota archaeon
MTKALEMTILDDGSESPISFKYLRKFNLAMGILHLVQGVAMLVLGFFWDFSRPLYSITLDYSGGPPVAALQPEFSFTAVGPTVAAFLLFSALAHLLIAGPLNKFYVKNLKKKMNPIRWFEYAFSSSIMVFFIAILFGVWDLWVLIGLFFLNMLMNLFGHMMELHNQTTKKTNWTAYIYGWIAGIIPWVIISVFFARIAINSTGMPWFVPVIYAFELILFMSFAFNMLLQYKKVGKWKDYLYGERMYQILSLVAKTLLAWLVFAGVFQPA

SEQ ID NO: 29 - protein, 268 aa, Halobacterium sp.DL1
MSSHSATTSSGRRESARDSRLRLWNSVMAVLHFLQGAAMVLLADTVLWPITRTRYGFDPGSQSIFPETVAFVDANLPLLVAGFLFISALAHTAIATVWYDKYVRYLDRGMNPYRWYEYSVSASLMIVVIGMLSGVWDLGTLVALFGLVAVMNLSGLLMEQRNELTEQTDWTPYWVGVIAGIVPWITIGVAFVGSVTASAGEFPEFVIYIYVSIFVFFNLFALNMALQYLEVSRWKNYLFGEKMYIVLSLVAKSALAWQVYFGTLNSPI

SEQ ID NO: 30 - protein, 253 aa, Thermoplasmatales archaeon
MTDYEDHTIRRFRIFNAIMGGIHLLQVFLVLYLSNNFSLPVTISKPVYNEFTNSISPVSETLFSIRVGPLVALFLFISAIAHILIATVLYYRYVENLKSGMNPYRWFEYSISASVMIVIIAMLTTIYDLGTLLALFTLTAVMNLMGLMMELHNQTTQNTDWTSYIIGCIAGLVPWIVIFIPLIAAESVPDFVIYIFVSIAIFFNCFAINMYLQYKKIGKWKDYLHGERVYIILSLVAKSALAWQVFAGTLRPM

SEQ ID NO: 31 - protein, 236 aa, synthetic construct
MVYSHADAAGWTLYWITYGIMAVTALIFFAMSLRRPIQQRSHHYTSFLIVAIASLAYYAMASQGGNTRIRVYQGPADSYRQIFWARYVNWFFTTPLLLLDLVLLSNLSKLRIAAIMVADIFMILTGLFGAVEARSNKWGWFVFGCIFMLYIFYELLVNVRKGAYARGGQHGMLYSVLLVWLLILWVQYPVVWGLAEGSSTVSSDTEIAWYAALDICAKCVFGFILLLGIESIDRKR

SEQ ID NO: 32 - protein, 284 aa, synthetic construct
MIHHDQVAEMLYGYAGAAAAKPTHTDSPGPIPTVIPTPPQFQEIGETGHRTLWVVFALMVLSSGFFAFMSWNVPISKRLYHVITTLITITASLSYFAMASGHVTSFSCTPAKDHHKHVPDVGYTECRQVFWGRYVNWAITTPLLLLDLSLLAGIDGAHTLMAVIADVIMVLSGLFASQGETATQRWGWYAIGCVSYLFVIWHVALHGARTVTAKGRGVTRLFSSLALFTFVLWTAYPIVWGIADGAHRTTVDTEILIYAVLDILAKPVFGLWLLFSHRSLAETN

SEQ ID NO: 33 - protein, 279 aa, synthetic construct
MIHPEQVADMLRPTTSTTSSHVPGPVPTVVPTPTEYQTLGETGHRTLWVVFALMVLSSGFFAFMSWNVPISKRLYHVITTLITITASLSYFAMASGHVTSFSCTPAKDHHKHVPDVGYTECRQVFWGRYVNWAITTPLLLLDLSLLAGIDGAHTLMAVIADVIMVLSGLFASQGETATQRWGWYAIGCVSYLFVIWHVALHGARTVTAKGRGVTRLFSSLALFTFVLWTAYPIVWGIADGAHRTTVDTEILIYAVLDILAKPVFGLWLLFSHRSLAETN

SEQ ID NO: 34 - protein, 238 aa, synthetic construct
MPEPGSEAIWLWLGTAGMFLGMLYFIARGWGETDSRRQKFYIATILITAIAFVNYLAMALGFGLTIVEFAGEEHPIYWARYSNWLFTTPLLLYDLGLLAGADRNTITSLVSLDVLMIGTGLVATLSPGSGVLSAGAERLVWWGISTAFLLVLLYFLFSSLSGRVADLPSDTRSTFKTLRNLVTVVWLVYPVWWLIGTEGIGLVGIGIETAGFMVIDLTAKVGFGIILLRSHGVLDGAA

SEQ ID NO: 35 - protein, 288 aa, synthetic construct
MIVDQFEEVLMKTSQLFPLPTATQSAQPTHVAPVPTVLPDTPIYETVGDSGSKTLWVVFVLMLIASAAFTALSWKIPVNRRLYHVITTIITLTAALSYFAMATGHGVALNKIVIRTQHDHVPDTYETVYRQVYYARYINWAITTPLLLLDLGLLAGMSGAHIFMAIVADLIMVLTGLFAAFGSEGTPQKWGWYTIACIAYIFVVWHLVLNGGANARVKGEKLRSFFVAIGAYTLILWTAYPIVWGLADGARKIGVDGEIIAYAVLDVLAKGVFGAWLLVTHANLRESD

SEQ ID NO: 36 - protein, 281 aa, synthetic construct
MIHPEQVADMLRPTTSTTSSHVPGPVPTVVPTPTEYQTLGETGHRTLWVTFALMVLSSGIFALLSWNVPTSKRLFHVITTLITVVASLSYFAMATGHATTFNCDTAWDHHKHVPDTSHQVCRQVFWGRYVNWALTTPLLLLELCLLAGVDGAHTLMAIVADVIMVLCGLFAALGEGGNTAQKWGWYTIGCFSYLFVIWHVALHGSRTVTAKGRGVSRLFTGLAVFALLLWTAYPIIWGIAGGARRTNVDTEILIYTVLDLLAKPVFGFWLLLSHRAMPETN

SEQ ID NO: 37 - protein, 235 aa, synthetic construct
MSNPNPFQTTLGTDAQWVVFAVMALAAIVFSIAVQFRPLPLRLTYYVNIAICTIAATAYYAMAVNGGDNKPTAGTGADERQVIYARYINWVFTTPLLLLDLVLLTNMPATMIAWIMGADIAMIAFGIIGAFTVGSYKWFYFVVGCIMLAVLAWGMINPIFKEELQKHKEYTGAYTTLLIYLIVLWVIYPIVWGLGAGGHIIGVDVEIIAMGVLDLLAKPLYAIGVLITVEVVYGK

SEQ ID NO: 38 - protein, 233 aa, synthetic construct
MAVHQIGEGGLVMYWVTFGLMAFSALAFAVMTFTRPLNKRSHGYITLAIVTIAAIAYYAMAASGGKALVSNPDGNLRDIYYARYINWFFTTPLLLLDIILLTGIPIGVTLWIVLADVAMIMLGLFGALSTNSYRWGYYGVSCAFFFVVLWGLFFPGAKGARARGGQVPGLYFGLAGYLALLWFGYPIVWGLAEGSDYISVTAEAASYAGLDIAAKVVFGWAVMLSHPLIARNQ

SEQ ID NO: 39 - protein, 257 aa, synthetic construct
MSSLFEKRNTAVATNYRGPTNSIVINEAGSDWYWAVFSVMAASAIVFSVMAAMTPRGERVFHYLTIAIVSVASVAYFTMAADLGSVAIISEFANYSSLPTRQVFYARYINWVITTPLLLTDLMLLAGLPWSTIIFTIVMDEVMVLTGLFGAITPSSYKWGYFTFGMVAYFFVAWVLIVEARKNAHRLGSDVHRLYIGIAIWTATLWTLYPVAWGLSEGGNVTSSDGEAIFYGVLDLLAKPVFGLWILLGHKGIGMDR

SEQ ID NO: 40 - protein, 263 aa, synthetic construct
MESNLVRRNGALTVNTMTQNNQSVAIHQTVRGSDWYYTVCAVMGTTSLAILALSRMKPRTDRVFFYLTSGLCMVACIAYFAMGSNLGWTPIDVEWLRNDSVVRGVNRQVFYARYINWVITTPMLLMNLLLTAGMPWPTILWIILLDEIMIVTGLIGALVKSRYKWGFYVFGCMAMFYIMWELAFPARKHAKVLGKDIHRSFVLCGVLTLVVWLCYPICWGLSEGGNVISPDSESVFYGVLDVLAKPSFSIALIATHWNIDPGR

SEQ ID NO: 41 - protein, 257 aa, synthetic construct
MGNSAIEVNGDTVDSYTADVKITTHGSDAYWAITAVMAFTTILFIAHSFTKPRTDRIFHYITISITLVASIAYFTMASNLGWASIFIEFQRDDPLVSGTTREIFYVRYINWVITTPLLLLNILLTAGLPWPTILFTILLDEVMIITGLVGALVKSSYKWGFFTFGCVAFFGVAWSVAWTGRKHANALGSDIGRVYLMTSVWTLFLWLLYPIAWGVSEGGNVISPDSEAAFYGTLDVLAKPIFGIILLWGHRNIPASR

SEQ ID NO: 42 - protein, 252 aa, synthetic construct
MDSLLLQRRNDAVATNPPNATFALTENGSSWLWAVFCVMALSCIIIAALSLFKPMGYRIFYLLNVAILATASVSYFSLASDLGLTPVTVEFRGPGTRQIAYVRYINWFVTTPLLLTELLLTAGLPTNIIISTIFADLVMIITGLAGALVVSRYKWGYYTMGCVAMLWVFWNVFTGIKVSGNIGPDVRKSYTLLAVWLMIIWLNYPICWGLAEGGNRITVVGEMVYYGVLDLLAKPVFAAISLAVHSKIELSR

SEQ ID NO: 43 - protein, 267 aa, synthetic construct
MDAILSKRNEVLSLNPLVANIDITTAASDWLWAVFAVMGLSAIILLVLGHATRPIGERAFHELAAALCFTASIAYYSMASDLGATPIEVEFIRGGTLGQNWVDIGVLRPTRSIWYARYINWTITTPLLLLELALTTALPLSQIFGLVFFDIVMIITGLLGALTASRYKWGFFVFGCVAMFWIFWVLFFPARKSASHLGTDYHRAYTSSAIVLCTLWTVYPIIWGVCDGGNVITPTSEMVAYGVLDLLAKPVFSFWHVFQLSRLDYAR

SEQ ID NO: 44 - protein, 256 aa, synthetic construct
MPFYIKNADIDITTHGSDWLWAVFSVMLLSAIGILVWGHVARPLGERAFHELAAALCFTASIAYFAMASDLGDVPIVVEFIRGGSLGQNWVQVGVENPTRAIWYARYINWTITTPMLLLELLLCTGLPLSQVFSVIFADLLMIETGLIGALVASRYKWGFYAFGCAAQLYIWWMLLVPGRRSAQHIGSDFAKSYTMSNIFLTTVWLVYPVIWGVADGGNVITPDSEMIAYGVLDLLAKPVFSVIHLMSLSKLDYAR

SEQ ID NO: 45 - protein, 248 aa, synthetic construct
MKLLLILGSVIALPTFAAGGGDLDASDYTGVSFWLVTAALLASTVFFFVERDRVSAKWKTSLTVSGLVTGIAFWHYMYMRGVWIETGDSPTVFRYINWLLTVPLLICEFYLILAAATNVAGSLFKKLLVGSLVMLVFGYMGEAGIMAAWPAFIIGCLAWVYMIYELWAGEGKSACNTASPAVQSAYNTMMYIIIFGWAIYPVGYFTGYLMGDGGSALNLNLIYNLADFVNKILFGLIIWNVAVKESSN

SEQ ID NO: 46 - protein, 250 aa, synthetic construct
MGKLLLILGSAIALPSFAAAGGDLDISDTVGVSFWLVTAGMLAATVFFFVERDQVSAKWKTSLTVSGLITGIAFWHYLYMRGVWIDTGDTPTVFRYINWLLTVPLQVVEFYLILAACTSVAASLFKKLLAGSLVMLGAGFAGEAGLAPVLPAFIIGMAGWLYMIYELYMGEGKAAVSTASPAVNSAYNAMMMIIVVGWAIYPAGYAAGYLMGGEGVYASNLNLIYNLADFVNKILFGLIIWNVAVKESSN

SEQ ID NO: 47 - protein, 242 aa, synthetic construct
MKFLLLLLADPTKLDPSDYVGFTFFVGAMAMMAASAFFFLSLNQFNKKWRTSVLVSGLITFIAAVHYWYMRDYWFAIQESPTFFRYVNWVLTVPLMCVEFYLILKVAGAKPALMWKLILFSVIMLVTGYFGEAVFQDQAALWGAISGAAYFYIVYEIWLGSAKKLAVAAGGDILKAHKILCWFVLVGWAIYPLGYMLGTDGWYTSILGKGSVDVAYNIADAINKIGFGLVIYALAVKKNEVD

SEQ ID NO: 48 - protein, 273 aa, synthetic construct
MTQELGNANFENFIGATEGFSEIAYQFTSHILTLGYAVMLAGLLYFILTIKNVDKKFQMSNILSAVVMVSAFLLLYAQAQNWTSSFTFNEEVGRYFLDPSGDLFNNGYRYLNWLINVPMLLFQILFVVSLTTSKFSSVRNQFWFSGAMMIITGYIGQFYEVSNLTAFLVWGAISSAFFFHILWVMKKVINEGKEGISPAGQKILSNIWILFLISWTLYPGAYLMPYLTGVDGFLYSEDGVMARQLVYTIADVSSKVIYGVLLGNLAITLSKNK

SEQ ID NO: 49 - protein, 275 aa, synthetic construct
MLMTVFSSAPELALLGSTFAQVDPSNLSVSDSLTYGQFNLVYNAFSFAIAAMFASALFFFSAQALVGQRYRLALLVSAIVVSIAGYHYFRIFNSWDAAYVLENGVYSLTSEKFNDAYRYVNWLLTVPLLLVETVAVLTLPAKEARPLLIKLTVASVLMIATGYPGEISDDITTRIIWGTVSTIPFAYILYVLWVELSRSLVRQPAAVQTLVRNMRWLLLLSWGVYPIAYLLPMLGVSGTSAAVGVQVGYTIADVLAKPVFGLLVFAIALVKTKAD

SEQ ID NO: 50 - protein, 258 aa, synthetic construct
MLQELPTLTPGQYSLVFNMFSFTVATMTASFVFFVLARNNVAPKYRISMMVSALVVFIAGYHYFRITSSWEAAYALQNGMYQPTGELFNDAYRYVNWLLTVPLLTVELVLVMGLPKNERGPLAAKLGFLAALMIVLGYPGEVSENAALFGTRGLWGFLSTIPFVWILYILFTQLGDTIQRQSSRVSTLLGNARLLLLATWGFYPIAYMIPMAFPEAFPSNTPGTIVALQVGYTIADVLAKAGYGVLIYNIAKAKSEEE

SEQ ID NO: 51 - protein, 256 aa, synthetic construct
MAKPTVKEIKSLQNFNRIAGVFHLLQMLAVLALANDFALPMTGTYLNGPPGTTFSAPVVILETPVGLAVALFLGLSALFHFIVSSGNFFKRYSASLMKNQNIFRWVQYSLSSSVMIVLIAQICGIADIVALLAIFGVNASMILFGWLQEKYTQPKDGDLLPFWFGCIAGIVPWIGLLIYVIAPGSTSDVAVPGFVYGIIISLFLFFNSFALVQYLQYKGKGKWSNYLRGERAYIVLSLVAKSALAWQIFSGTLIPA

SEQ ID NO: 52 - protein, 238 aa, synthetic construct
MPEPGSEAIWLWLGTAGMFLGMLYFIARGWGETDSRRQKFYIATILITAIAFVNYLAMALGFGLTIVEFAGEEHPIYWARYSDWLFTTPLLLYNLGLLAGADRNTITSLVSLDVLMIGTGLVATLSPGSGVLSAGAERLVWWGISTAFLLVLLYFLFSSLSGRVADLPSDTRSTFKTLRNLVTVVWLVYPVWWLIGTEGIGLVGIGIVTAGFMVIDLTAKVGFGIILLRSHGVLDGAA

SEQ ID NO: 53 - protein, 288 aa, synthetic construct
MIVDQFEEVLMKTSQLFPLPTATQSAQPTHVAPVPTVLPDTPIYETVGDSGSKTLWVVFVLMLIASAAFTALSWKIPVNRRLYHVITTIITLTAALSYFAMATGHGVALNKIVIRTQHDHVPDTYETVYRQVYYARYIDWAITTPLLLLNLGLLAGMSGAHIFMAIVADLIMVLTGLFAAFGSEGTPQKWGWYTIACIAYIFVVWHLVLNGGANARVKGEKLRSFFVAIGAYTLILWTAYPIVWGLADGARKIGVDGVIIAYAVLDVLAKGVFGAWLLVTHANLRESD

SEQ ID NO: 54 - protein, 284 aa, synthetic construct
MIHHDQVAEMLYGYAGAAAAKPTHTDSPGPIPTVIPTPPQFQEIGETGHRTLWVVFALMVLSSGFFAFMSWNVPISKRLYHVITTLITITASLSYFAMASGHVTSFSCTPAKDHHKHVPDVGYTECRQVFWGRYVDWAITTPLLLLNLSLLAGIDGAHTLMAVIADVIMVLSGLFASQGETATQRWGWYAIGCVSYLFVIWHVALHGARTVTAKGRGVTRLFSSLALFTFVLWTAYPIVWGIADGAHRTTVDTVILIYAVLDILAKPVFGLWLLFSHRSLAETN

SEQ ID NO: 55 - protein, 281 aa, synthetic construct
MIHPEQVADMLRPTTSTTSSHVPGPVPTVVPTPTEYQTLGETGHRTLWVTFALMVLSSGIFALLSWNVPTSKRLFHVITTLITVVASLSYFAMATGHATTFNCDTAWDHHKHVPDTSHQVCRQVFWGRYVDWALTTPLLLLQLCLLAGVDGAHTLMAIVADVIMVLCGLFAALGEGGNTAQKWGWYTIGCFSYLFVIWHVALHGSRTVTAKGRGVSRLFTGLAVFALLLWTAYPIIWGIAGGARRTNVDTVILIYTVLDLLAKPVFGFWLLLSHRAMPETN

SEQ ID NO: 56 - protein, 235 aa, synthetic construct
MSNPNPFQTTLGTDAQWVVFAVMALAAIVFSIAVQFRPLPLRLTYYVNIAICTIAATAYYAMAVNGGDNKPTAGTGADERQVIYARYIDWVFTTPLLLLNLVLLTNMPATMIAWIMGADIAMIAFGIIGAFTVGSYKWFYFVVGCIMLAVLAWGMINPIFKEELQKHKEYTGAYTTLLIYLIVLWVIYPIVWGLGAGGHIIGVDVVIIAMGVLDLLAKPLYAIGVLITVEVVYGK

SEQ ID NO: 57 - protein, 233 aa, synthetic construct
MAVHQIGEGGLVMYWVTFGLMAFSALAFAVMTFTRPLNKRSHGYITLAIVTIAAIAYYAMAASGGKALVSNPDGNLRDIYYARYIDWFFTTPLLLLNIILLTGIPIGVTLWIVLADVAMIMLGLFGALSTNSYRWGYYGVSCAFFFVVLWGLFFPGAKGARARGGQVPGLYFGLAGYLALLWFGYPIVWGLAEGSDYISVTAVAASYAGLDIAAKVVFGWAVMLSHPLIARNQ

SEQ ID NO: 58 - protein, 236 aa, synthetic construct
MVYSHADAAGWTLYWITYGIMAVTALIFFAMSLRRPIQQRSHHYTSFLIVAIASLAYYAMASQGGNTRIRVYQGPADSYRQIFWARYVDWFFTTPLLLLNLVLLSNLSKLRIAAIMVADIFMILTGLFGAVEARSNKWGWFVFGCIFMLYIFYELLVNVRKGAYARGGQHGMLYSVLLVWLLILWVQYPVVWGLAEGSSTVSSDTVIAWYAALDICAKCVFGFILLLGIESIDRKR

SEQ ID NO: 59 - protein, 257 aa, synthetic construct
MSSLFEKRNTAVATNYRGPTNSIVINEAGSDWYWAVFSVMAASAIVFSVMAAMTPRGERVFHYLTIAIVSVASVAYFTMAADLGSVAIISEFANYSSLPTRQVFYARYIDWVITTPLLLTNLMLLAGLPWSTIIFTIVMDEVMVLTGLFGAITPSSYKWGYFTFGMVAYFFVAWVLIVEARKNAHRLGSDVHRLYIGIAIWTATLWTLYPVAWGLSEGGNVTSSDGVAIFYGVLDLLAKPVFGLWILLGHKGIGMDR

SEQ ID NO: 60 - protein, 263 aa, synthetic construct
MESNLVRRNGALTVNTMTQNNQSVAIHQTVRGSDWYYTVCAVMGTTSLAILALSRMKPRTDRVFFYLTSGLCMVACIAYFAMGSNLGWTPIDVEWLRNDSVVRGVNRQVFYARYIDWVITTPMLLMNLLLTAGMPWPTILWIILLDEIMIVTGLIGALVKSRYKWGFYVFGCMAMFYIMWELAFPARKHAKVLGKDIHRSFVLCGVLTLVVWLCYPICWGLSEGGNVISPDSVSVFYGVLDVLAKPSFSIALIATHWNIDPGR

SEQ ID NO: 61 - protein, 257 aa, synthetic construct
MGNSAIEVNGDTVDSYTADVKITTHGSDAYWAITAVMAFTTILFIAHSFTKPRTDRIFHYITISITLVASIAYFTMASNLGWASIFIEFQRDDPLVSGTTREIFYVRYIDWVITTPLLLLNILLTAGLPWPTILFTILLDEVMIITGLVGALVKSSYKWGFFTFGCVAFFGVAWSVAWTGRKHANALGSDIGRVYLMTSVWTLFLWLLYPIAWGVSEGGNVISPDSVAAFYGTLDVLAKPIFGIILLWGHRNIPASR

SEQ ID NO: 62 - protein, 252 aa, synthetic construct
MDSLLLQRRNDAVATNPPNATFALTENGSSWLWAVFCVMALSCIIIAALSLFKPMGYRIFYLLNVAILATASVSYFSLASDLGLTPVTVEFRGPGTRQIAYVRYIDWFVTTPLLLTQLLLTAGLPTNIIISTIFADLVMIITGLAGALVVSRYKWGYYTMGCVAMLWVFWNVFTGIKVSGNIGPDVRKSYTLLAVWLMIIWLNYPICWGLAEGGNRITVVGVMVYYGVLDLLAKPVFAAISLAVHSKIELSR

SEQ ID NO: 63 - protein, 267 aa, synthetic construct
MDAILSKRNEVLSLNPLVANIDITTAASDWLWAVFAVMGLSAIILLVLGHATRPIGERAFHELAAALCFTASIAYYSMASDLGATPIEVEFIRGGTLGQNWVDIGVLRPTRSIWYARYIDWTITTPLLLLQLALTTALPLSQIFGLVFFDIVMIITGLLGALTASRYKWGFFVFGCVAMFWIFWVLFFPARKSASHLGTDYHRAYTSSAIVLCTLWTVYPIIWGVCDGGNVITPTSVMVAYGVLDLLAKPVFSFWHVFQLSRLDYAR

SEQ ID NO: 64 - protein, 256 aa, synthetic construct
MPFYIKNADIDITTHGSDWLWAVFSVMLLSAIGILVWGHVARPLGERAFHELAAALCFTASIAYFAMASDLGDVPIVVEFIRGGSLGQNWVQVGVENPTRAIWYARYIDWTITTPMLLLQLLLCTGLPLSQVFSVIFADLLMIETGLIGALVASRYKWGFYAFGCAAQLYIWWMLLVPGRRSAQHIGSDFAKSYTMSNIFLTTVWLVYPVIWGVADGGNVITPDSVMIAYGVLDLLAKPVFSVIHLMSLSKLDYAR

SEQ ID NO: 65 - protein, 242 aa, synthetic construct
MKFLLLLLADPTKLDPSDYVGFTFFVGAMAMMAASAFFFLSLNQFNKKWRTSVLVSGLITFIAAVHYWYMRDYWFAIQESPTFFRYVDWVLTVPLMCVQFYLILKVAGAKPALMWKLILFSVIMLVTGYFGEAVFQDQAALWGAISGAAYFYIVYEIWLGSAKKLAVAAGGDILKAHKILCWFVLVGWAIYPLGYMLGTDGWYTSILGKGSVDVAYNIADAINKIGFGLVIYALAVKKNEVD

SEQ ID NO: 66 - protein, 273 aa, synthetic construct
MTQELGNANFENFIGATEGFSEIAYQFTSHILTLGYAVMLAGLLYFILTIKNVDKKFQMSNILSAVVMVSAFLLLYAQAQNWTSSFTFNEEVGRYFLDPSGDLFNNGYRYLNWLIDVPMLLFQILFVVSLTTSKFSSVRNQFWFSGAMMIITGYIGQFYEVSNLTAFLVWGAISSAFFFHILWVMKKVINEGKEGISPAGQKILSNIWILFLISWTLYPGAYLMPYLTGVDGFLYSEDGVMARQLVYTIANVSSKVIYGVLLGNLAITLSKNK

SEQ ID NO: 67 - protein, 275 aa, synthetic construct
MLMTVFSSAPELALLGSTFAQVDPSNLSVSDSLTYGQFNLVYNAFSFAIAAMFASALFFFSAQALVGQRYRLALLVSAIVVSIAGYHYFRIFNSWDAAYVLENGVYSLTSEKFNDAYRYVDWLLTVPLLLVQTVAVLTLPAKEARPLLIKLTVASVLMIATGYPGEISDDITTRIIWGTVSTIPFAYILYVLWVELSRSLVRQPAAVQTLVRNMRWLLLLSWGVYPIAYLLPMLGVSGTSAAVGVQVGYTIADVLAKPVFGLLVFAIALVKTKAD

SEQ ID NO: 68 - protein, 258 aa, synthetic construct
MLQELPTLTPGQYSLVFNMFSFTVATMTASFVFFVLARNNVAPKYRISMMVSALVVFIAGYHYFRITSSWEAAYALQNGMYQPTGELFNDAYRYVDWLLTVPLLTVQLVLVMGLPKNERGPLAAKLGFLAALMIVLGYPGEVSENAALFGTRGLWGFLSTIPFVWILYILFTQLGDTIQRQSSRVSTLLGNARLLLLATWGFYPIAYMIPMAFPEAFPSNTPGTIVALQVGYTIADVLAKAGYGVLIYNIAKAKSEEE

SEQ ID NO: 69 - protein, 267 aa, synthetic construct
MTMLEHLEGTMDGWYAENDLGQGAIIAHWVTFFFHMITTFYLGYVSFHSKGPGGKQPYFAGYHEENNIGIFVNLFAAISYFGKVVSDTHGHNYQNVGPFIIGLGNYRYADYMLTCPLLVMNLLFQLRAPYKITCAMLIFAVLMIGAVTNFYPGDDMKGPAVAWFCFGCFWYLIAYIFMAHIVSKQYGRLDYLAHGTKAEGALFSLKLAIITFFAIWVAFPLVWLLSVGTGVLSNEAAEICHCICDVVAKSVYGFALANFREQYDREL

SEQ ID NO: 70 - protein, 241 aa, synthetic construct
MVESSAVIAANWISFLVIAGSFVVLCFISLRYKGPGGNENYYNGFREQNMLTVIINLWCALAYFAKVLQSHSDDDGFVPLTKIPYLDYATTCPLLTLNLMWCLDAPYKITSAVLVFTVMITGVACSLAVAPYSFYWFAMGMVLFIFTYVLMLSIVRERLEFITQCAHDSNAKRSIKHLKAAVIIYFGIWPIFAILWLLSYRAANVISNDTNHILHCILDVIAKSCFGFVLLHFKMYFDKKL

SEQ ID NO: 71 - protein, 241 aa, synthetic construct
MVASSAVITANWISFLAISASFIILLVISLRYKGPGGTESFYNGFKEQNMLTVFINLWCALAYFAKVLQSHSNDNGFAPLTVIPYVDYCTTCPLLTLNLLWCLDAPYKISSAVLVFTCLVIAVACSLAVAPFSYCWFAMGMVLFTFTYVFILSIVRQRLDFFTLCARDSNAKQSLKHLKTAVFIYFGIWLLFPLLWLLSYRAANVISNDINHIFHCILDVIAKSVYGFALLYFKMYFDKKL

SEQ ID NO: 72 - protein, 247 aa, synthetic construct
MVSALDQNGPQYLQNPIVIAADWIGFIALFGSSLAVAYKLVTFKGPDQDDVYFFGYREEKMISVFVNLFAALAYWAKLASHANGDVGPAASVTTYKYLDYLFTCPLLTINLLWCLNLPYKFTFGAIVAVCILCAFMASVIPPPARYMWFGMGITVFSAAWFNILKLVRMRLEQFVSKEAKKVRQSLKVACMTYFFIWLGYPTLWVLGDAGVLDSVVSALLHTFLDVFSKSIYGFALLHFVMRTDKRE

SEQ ID NO: 73 - protein, 260 aa, synthetic construct
MTTSAPSLSDPNWQYGMGGWNNPRLPNFNLHDPTVIGVDWLGFLCLLGASLALMYKLMSFKGPDGDQEFFVGYREEKCLSIYVNLIAAITYWGRICAHFNNDMGLSLSVNYFKYLDYIFTCPILTLNLLWSLNLPYKITYSLFVGLTIACNAFEPPARYLWFMFGCFIFAFTWISIIRLVYARFQQFLNEDAKKIRAPLKLSLTLYFSIWCGYPALWLLTEFGAISQLAAHVMTVIMDVAAKSVYGFALLKFQLGVDKRD

SEQ ID NO: 74 - protein, 261 aa, synthetic construct
MSTSSVAYLRTPVVQALDWVGFISLGGTAAVLAYRLMNFKPPNKDILYFFGYREKGMISLYVNLFAAVAYYARITSHLSGDVGAATNIILYKYFDYLITCPLLTFNLLTTLNLPYKITYAVYVQITIFTGFMSANTPPPATFLWFAFGMLLFSYTWFNIISLVQVRFIQYFAKKGNTTQSRRVSVASKAGFRNKNVRNPLQTALSTYFCIWMVYPVLWLLLKTKVIDQVTEHCINVVMDVLAKSMYGFALLRFQLLMDKAN

SEQ ID NO: 75 - protein, 298 aa, synthetic construct
IGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTT

SEQ ID NO: 76 - protein, 225 aa, synthetic construct
IGTMRSKLYLEGSVNGHQFKCTHEGEGKPYEGKQTNRIKVVEGGPLPFAFDILATHFMYGSKVFIKYPADLPDYFKQSFPEGFTWERVMVFEDGGVLTATQDTSLQDGELIYNVKVRGVNFPANGPVMQKKTLGWEPSTETMYPADGGLEGRCDKALKLVGGGHLHVNFKTTYKSKKPVKMPGVHYVDRRLERIKEADNETYVEQYEHAVARYSNLGGGMDELYK

SEQ ID NO: 77 - protein, 226 aa, synthetic construct
IGTKEFMRFKVRMEGSVNGHEFEIEGEGEGRPYEGFQTAKLKVTKGGPLPFAWDILSPHFTYGSKAYVKHPADIPDYFKLSFPEGFKWERVMNYEDGGVVTVTQDSSLQDGEFIYKVKLRGTNFPSDGPVMQKKTMGWEASSERMYPEDGALKGKIKMRLKLKDGGHYTSEVKTTYKAKKPVQLPGAYIVDIKLDITSHNEDYTIVEQYERAEGRHSTGGMDELYK

SEQ ID NO: 78 - protein, 230 aa, synthetic construct
IGTVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSKLSKDPNEKRDHMVLLEFVTAAGITLGMDELYK

SEQ ID NO: 79 - protein, 39 aa, synthetic construct
MIHPEQVADMLRPTTSTTSSHVPGPVPTVVPTPTEYQTL

SEQ ID NO: 80 - protein, 20 aa, synthetic construct
KSRITSEGEYIPLDQIDINV

SEQ ID NO: 81 - protein, 7 aa, synthetic construct
FCYENEV

SEQ ID NO: 82 - protein, 65 aa, synthetic construct
QSQPILNTKEMAPQSKPPEELEMSSMPSPVAPLPARTEGVIDMRSMSSIDSFISCATDFPEATRF

SEQ ID NO: 83 - protein, 561 aa, synthetic construct
MVYSHADAAGWTLYWITYGIMAVTALIFFAMSLRRPIQQRSHHYTSFLIVAIASLAYYAMASQGGNTRIRVYQGPADSYRQIFWARYVNWFFTTPLLLLDLVLLSNLSKLRIAAIMVADIFMILTGLFGAVEARSNKWGWFVFGCIFMLYIFYELLVNVRKGAYARGGQHGMLYSVLLVWLLILWVQYPVVWGLAEGSSTVSSDTEIAWYAALDICAKCVFGFILLLGIESIDRKRIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTKSRITSEGEYIPLDQIDINVFCYENEV

SEQ ID NO: 84 - protein, 626 aa, synthetic construct
MVYSHADAAGWTLYWITYGIMAVTALIFFAMSLRRPIQQRSHHYTSFLIVAIASLAYYAMASQGGNTRIRVYQGPADSYRQIFWARYVNWFFTTPLLLLDLVLLSNLSKLRIAAIMVADIFMILTGLFGAVEARSNKWGWFVFGCIFMLYIFYELLVNVRKGAYARGGQHGMLYSVLLVWLLILWVQYPVVWGLAEGSSTVSSDTEIAWYAALDICAKCVFGFILLLGIESIDRKRIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTKSRITSEGEYIPLDQIDINVFCYENEVQSQPILNTKEMAPQSKPPEELEMSSMPSPVAPLPARTEGVIDMRSMSSIDSFISCATDFPEATRF

SEQ ID NO: 85 - protein, 488 aa, synthetic construct
MVYSHADAAGWTLYWITYGIMAVTALIFFAMSLRRPIQQRSHHYTSFLIVAIASLAYYAMASQGGNTRIRVYQGPADSYRQIFWARYVNWFFTTPLLLLDLVLLSNLSKLRIAAIMVADIFMILTGLFGAVEARSNKWGWFVFGCIFMLYIFYELLVNVRKGAYARGGQHGMLYSVLLVWLLILWVQYPVVWGLAEGSSTVSSDTEIAWYAALDICAKCVFGFILLLGIESIDRKRIGTMRSKLYLEGSVNGHQFKCTHEGEGKPYEGKQTNRIKVVEGGPLPFAFDILATHFMYGSKVFIKYPADLPDYFKQSFPEGFTWERVMVFEDGGVLTATQDTSLQDGELIYNVKVRGVNFPANGPVMQKKTLGWEPSTETMYPADGGLEGRCDKALKLVGGGHLHVNFKTTYKSKKPVKMPGVHYVDRRLERIKEADNETYVEQYEHAVARYSNLGGGMDELYKKSRITSEGEYIPLDQIDINVFCYENEV

SEQ ID NO: 86 - protein, 489 aa, synthetic construct
MVYSHADAAGWTLYWITYGIMAVTALIFFAMSLRRPIQQRSHHYTSFLIVAIASLAYYAMASQGGNTRIRVYQGPADSYRQIFWARYVNWFFTTPLLLLDLVLLSNLSKLRIAAIMVADIFMILTGLFGAVEARSNKWGWFVFGCIFMLYIFYELLVNVRKGAYARGGQHGMLYSVLLVWLLILWVQYPVVWGLAEGSSTVSSDTEIAWYAALDICAKCVFGFILLLGIESIDRKRIGTKEFMRFKVRMEGSVNGHEFEIEGEGEGRPYEGFQTAKLKVTKGGPLPFAWDILSPHFTYGSKAYVKHPADIPDYFKLSFPEGFKWERVMNYEDGGVVTVTQDSSLQDGEFIYKVKLRGTNFPSDGPVMQKKTMGWEASSERMYPEDGALKGKIKMRLKLKDGGHYTSEVKTTYKAKKPVQLPGAYIVDIKLDITSHNEDYTIVEQYERAEGRHSTGGMDELYKKSRITSEGEYIPLDQIDINVFCYENEV

SEQ ID NO: 87 - protein, 493 aa, synthetic construct
MVYSHADAAGWTLYWITYGIMAVTALIFFAMSLRRPIQQRSHHYTSFLIVAIASLAYYAMASQGGNTRIRVYQGPADSYRQIFWARYVNWFFTTPLLLLDLVLLSNLSKLRIAAIMVADIFMILTGLFGAVEARSNKWGWFVFGCIFMLYIFYELLVNVRKGAYARGGQHGMLYSVLLVWLLILWVQYPVVWGLAEGSSTVSSDTEIAWYAALDICAKCVFGFILLLGIESIDRKRIGTVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSKLSKDPNEKRDHMVLLEFVTAAGITLGMDELYKKSRITSEGEYIPLDQIDINVFCYENEV

SEQ ID NO: 88 - protein, 609 aa, synthetic construct
MIHHDQVAEMLYGYAGAAAAKPTHTDSPGPIPTVIPTPPQFQEIGETGHRTLWVVFALMVLSSGFFAFMSWNVPISKRLYHVITTLITITASLSYFAMASGHVTSFSCTPAKDHHKHVPDVGYTECRQVFWGRYVNWAITTPLLLLDLSLLAGIDGAHTLMAVIADVIMVLSGLFASQGETATQRWGWYAIGCVSYLFVIWHVALHGARTVTAKGRGVTRLFSSLALFTFVLWTAYPIVWGIADGAHRTTVDTEILIYAVLDILAKPVFGLWLLFSHRSLAETNIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTKSRITSEGEYIPLDQIDINVFCYENEV

SEQ ID NO: 89 - protein, 674 aa, synthetic construct
MIHHDQVAEMLYGYAGAAAAKPTHTDSPGPIPTVIPTPPQFQEIGETGHRTLWVVFALMVLSSGFFAFMSWNVPISKRLYHVITTLITITASLSYFAMASGHVTSFSCTPAKDHHKHVPDVGYTECRQVFWGRYVNWAITTPLLLLDLSLLAGIDGAHTLMAVIADVIMVLSGLFASQGETATQRWGWYAIGCVSYLFVIWHVALHGARTVTAKGRGVTRLFSSLALFTFVLWTAYPIVWGIADGAHRTTVDTEILIYAVLDILAKPVFGLWLLFSHRSLAETNIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTKSRITSEGEYIPLDQIDINVFCYENEVQSQPILNTKEMAPQSKPPEELEMSSMPSPVAPLPARTEGVIDMRSMSSIDSFISCATDFPEATRF

SEQ ID NO: 90 - protein, 669 aa, synthetic construct
MIHPEQVADMLRPTTSTTSSHVPGPVPTVVPTPTEYQTLGETGHRTLWVVFALMVLSSGFFAFMSWNVPISKRLYHVITTLITITASLSYFAMASGHVTSFSCTPAKDHHKHVPDVGYTECRQVFWGRYVNWAITTPLLLLDLSLLAGIDGAHTLMAVIADVIMVLSGLFASQGETATQRWGWYAIGCVSYLFVIWHVALHGARTVTAKGRGVTRLFSSLALFTFVLWTAYPIVWGIADGAHRTTVDTEILIYAVLDILAKPVFGLWLLFSHRSLAETNIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTKSRITSEGEYIPLDQIDINVFCYENEVQSQPILNTKEMAPQSKPPEELEMSSMPSPVAPLPARTEGVIDMRSMSSIDSFISCATDFPEATRF

SEQ ID NO: 91 - protein, 563 aa, synthetic construct
MPEPGSEAIWLWLGTAGMFLGMLYFIARGWGETDSRRQKFYIATILITAIAFVNYLAMALGFGLTIVEFAGEEHPIYWARYSNWLFTTPLLLYDLGLLAGADRNTITSLVSLDVLMIGTGLVATLSPGSGVLSAGAERLVWWGISTAFLLVLLYFLFSSLSGRVADLPSDTRSTFKTLRNLVTVVWLVYPVWWLIGTEGIGLVGIGIETAGFMVIDLTAKVGFGIILLRSHGVLDGAAIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTKSRITSEGEYIPLDQIDINVFCYENEV

SEQ ID NO: 92 - protein, 613 aa, synthetic construct
MIVDQFEEVLMKTSQLFPLPTATQSAQPTHVAPVPTVLPDTPIYETVGDSGSKTLWVVFVLMLIASAAFTALSWKIPVNRRLYHVITTIITLTAALSYFAMATGHGVALNKIVIRTQHDHVPDTYETVYRQVYYARYINWAITTPLLLLDLGLLAGMSGAHIFMAIVADLIMVLTGLFAAFGSEGTPQKWGWYTIACIAYIFVVWHLVLNGGANARVKGEKLRSFFVAIGAYTLILWTAYPIVWGLADGARKIGVDGEIIAYAVLDVLAKGVFGAWLLVTHANLRESDIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTKSRITSEGEYIPLDQIDINVFCYENEV

SEQ ID NO: 93 - protein, 606 aa, synthetic construct
MIHPEQVADMLRPTTSTTSSHVPGPVPTVVPTPTEYQTLGETGHRTLWVTFALMVLSSGIFALLSWNVPTSKRLFHVITTLITVVASLSYFAMATGHATTFNCDTAWDHHKHVPDTSHQVCRQVFWGRYVNWALTTPLLLLELCLLAGVDGAHTLMAIVADVIMVLCGLFAALGEGGNTAQKWGWYTIGCFSYLFVIWHVALHGSRTVTAKGRGVSRLFTGLAVFALLLWTAYPIIWGIAGGARRTNVDTEILIYTVLDLLAKPVFGFWLLLSHRAMPETNIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTKSRITSEGEYIPLDQIDINVFCYENEV

SEQ ID NO: 94 - protein, 560 aa, synthetic construct
MSNPNPFQTTLGTDAQWVVFAVMALAAIVFSIAVQFRPLPLRLTYYVNIAICTIAATAYYAMAVNGGDNKPTAGTGADERQVIYARYINWVFTTPLLLLDLVLLTNMPATMIAWIMGADIAMIAFGIIGAFTVGSYKWFYFVVGCIMLAVLAWGMINPIFKEELQKHKEYTGAYTTLLIYLIVLWVIYPIVWGLGAGGHIIGVDVEIIAMGVLDLLAKPLYAIGVLITVEVVYGKIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTKSRITSEGEYIPLDQIDINVFCYENEV

SEQ ID NO: 95 - protein, 558 aa, synthetic construct
MAVHQIGEGGLVMYWVTFGLMAFSALAFAVMTFTRPLNKRSHGYITLAIVTIAAIAYYAMAASGGKALVSNPDGNLRDIYYARYINWFFTTPLLLLDIILLTGIPIGVTLWIVLADVAMIMLGLFGALSTNSYRWGYYGVSCAFFFVVLWGLFFPGAKGARARGGQVPGLYFGLAGYLALLWFGYPIVWGLAEGSDYISVTAEAASYAGLDIAAKVVFGWAVMLSHPLIARNQIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTKSRITSEGEYIPLDQIDINVFCYENEV

SEQ ID NO: 96 - protein, 582 aa, synthetic construct
MSSLFEKRNTAVATNYRGPTNSIVINEAGSDWYWAVFSVMAASAIVFSVMAAMTPRGERVFHYLTIAIVSVASVAYFTMAADLGSVAIISEFANYSSLPTRQVFYARYINWVITTPLLLTDLMLLAGLPWSTIIFTIVMDEVMVLTGLFGAITPSSYKWGYFTFGMVAYFFVAWVLIVEARKNAHRLGSDVHRLYIGIAIWTATLWTLYPVAWGLSEGGNVTSSDGEAIFYGVLDLLAKPVFGLWILLGHKGIGMDRIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTKSRITSEGEYIPLDQIDINVFCYENEV

SEQ ID NO: 97 - protein, 588 aa, synthetic construct
MESNLVRRNGALTVNTMTQNNQSVAIHQTVRGSDWYYTVCAVMGTTSLAILALSRMKPRTDRVFFYLTSGLCMVACIAYFAMGSNLGWTPIDVEWLRNDSVVRGVNRQVFYARYINWVITTPMLLMNLLLTAGMPWPTILWIILLDEIMIVTGLIGALVKSRYKWGFYVFGCMAMFYIMWELAFPARKHAKVLGKDIHRSFVLCGVLTLVVWLCYPICWGLSEGGNVISPDSESVFYGVLDVLAKPSFSIALIATHWNIDPGRIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTKSRITSEGEYIPLDQIDINVFCYENEV

SEQ ID NO: 98 - protein, 582 aa, synthetic construct
MGNSAIEVNGDTVDSYTADVKITTHGSDAYWAITAVMAFTTILFIAHSFTKPRTDRIFHYITISITLVASIAYFTMASNLGWASIFIEFQRDDPLVSGTTREIFYVRYINWVITTPLLLLNILLTAGLPWPTILFTILLDEVMIITGLVGALVKSSYKWGFFTFGCVAFFGVAWSVAWTGRKHANALGSDIGRVYLMTSVWTLFLWLLYPIAWGVSEGGNVISPDSEAAFYGTLDVLAKPIFGIILLWGHRNIPASRIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTKSRITSEGEYIPLDQIDINVFCYENEV

SEQ ID NO: 99 - protein, 577 aa, synthetic construct
MDSLLLQRRNDAVATNPPNATFALTENGSSWLWAVFCVMALSCIIIAALSLFKPMGYRIFYLLNVAILATASVSYFSLASDLGLTPVTVEFRGPGTRQIAYVRYINWFVTTPLLLTELLLTAGLPTNIIISTIFADLVMIITGLAGALVVSRYKWGYYTMGCVAMLWVFWNVFTGIKVSGNIGPDVRKSYTLLAVWLMIIWLNYPICWGLAEGGNRITVVGEMVYYGVLDLLAKPVFAAISLAVHSKIELSRIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTKSRITSEGEYIPLDQIDINVFCYENEV

SEQ ID NO: 100 - protein, 592 aa, synthetic construct
MDAILSKRNEVLSLNPLVANIDITTAASDWLWAVFAVMGLSAIILLVLGHATRPIGERAFHELAAALCFTASIAYYSMASDLGATPIEVEFIRGGTLGQNWVDIGVLRPTRSIWYARYINWTITTPLLLLELALTTALPLSQIFGLVFFDIVMIITGLLGALTASRYKWGFFVFGCVAMFWIFWVLFFPARKSASHLGTDYHRAYTSSAIVLCTLWTVYPIIWGVCDGGNVITPTSEMVAYGVLDLLAKPVFSFWHVFQLSRLDYARIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTKSRITSEGEYIPLDQIDINVFCYENEV

SEQ ID NO: 101 - protein, 581 aa, synthetic construct
MPFYIKNADIDITTHGSDWLWAVFSVMLLSAIGILVWGHVARPLGERAFHELAAALCFTASIAYFAMASDLGDVPIVVEFIRGGSLGQNWVQVGVENPTRAIWYARYINWTITTPMLLLELLLCTGLPLSQVFSVIFADLLMIETGLIGALVASRYKWGFYAFGCAAQLYIWWMLLVPGRRSAQHIGSDFAKSYTMSNIFLTTVWLVYPVIWGVADGGNVITPDSEMIAYGVLDLLAKPVFSVIHLMSLSKLDYARIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTKSRITSEGEYIPLDQIDINVFCYENEV

SEQ ID NO: 102 - protein, 573 aa, synthetic construct
MKLLLILGSVIALPTFAAGGGDLDASDYTGVSFWLVTAALLASTVFFFVERDRVSAKWKTSLTVSGLVTGIAFWHYMYMRGVWIETGDSPTVFRYINWLLTVPLLICEFYLILAAATNVAGSLFKKLLVGSLVMLVFGYMGEAGIMAAWPAFIIGCLAWVYMIYELWAGEGKSACNTASPAVQSAYNTMMYIIIFGWAIYPVGYFTGYLMGDGGSALNLNLIYNLADFVNKILFGLIIWNVAVKESSNIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTKSRITSEGEYIPLDQIDINVFCYENEV

SEQ ID NO: 103 - protein, 575 aa, synthetic construct
MGKLLLILGSAIALPSFAAAGGDLDISDTVGVSFWLVTAGMLAATVFFFVERDQVSAKWKTSLTVSGLITGIAFWHYLYMRGVWIDTGDTPTVFRYINWLLTVPLQVVEFYLILAACTSVAASLFKKLLAGSLVMLGAGFAGEAGLAPVLPAFIIGMAGWLYMIYELYMGEGKAAVSTASPAVNSAYNAMMMIIVVGWAIYPAGYAAGYLMGGEGVYASNLNLIYNLADFVNKILFGLIIWNVAVKESSNIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTKSRITSEGEYIPLDQIDINVFCYENEV

SEQ ID NO: 104 - protein, 567 aa, synthetic construct
MKFLLLLLADPTKLDPSDYVGFTFFVGAMAMMAASAFFFLSLNQFNKKWRTSVLVSGLITFIAAVHYWYMRDYWFAIQESPTFFRYVNWVLTVPLMCVEFYLILKVAGAKPALMWKLILFSVIMLVTGYFGEAVFQDQAALWGAISGAAYFYIVYEIWLGSAKKLAVAAGGDILKAHKILCWFVLVGWAIYPLGYMLGTDGWYTSILGKGSVDVAYNIADAINKIGFGLVIYALAVKKNEVDIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTKSRITSEGEYIPLDQIDINVFCYENEV

SEQ ID NO: 105 - protein, 598 aa, synthetic construct
MTQELGNANFENFIGATEGFSEIAYQFTSHILTLGYAVMLAGLLYFILTIKNVDKKFQMSNILSAVVMVSAFLLLYAQAQNWTSSFTFNEEVGRYFLDPSGDLFNNGYRYLNWLINVPMLLFQILFVVSLTTSKFSSVRNQFWFSGAMMIITGYIGQFYEVSNLTAFLVWGAISSAFFFHILWVMKKVINEGKEGISPAGQKILSNIWILFLISWTLYPGAYLMPYLTGVDGFLYSEDGVMARQLVYTIADVSSKVIYGVLLGNLAITLSKNKIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTKSRITSEGEYIPLDQIDINVFCYENEV

SEQ ID NO: 106 - protein, 600 aa, synthetic construct
MLMTVFSSAPELALLGSTFAQVDPSNLSVSDSLTYGQFNLVYNAFSFAIAAMFASALFFFSAQALVGQRYRLALLVSAIVVSIAGYHYFRIFNSWDAAYVLENGVYSLTSEKFNDAYRYVNWLLTVPLLLVETVAVLTLPAKEARPLLIKLTVASVLMIATGYPGEISDDITTRIIWGTVSTIPFAYILYVLWVELSRSLVRQPAAVQTLVRNMRWLLLLSWGVYPIAYLLPMLGVSGTSAAVGVQVGYTIADVLAKPVFGLLVFAIALVKTKADIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTKSRITSEGEYIPLDQIDINVFCYENEV

SEQ ID NO: 107 - protein, 583 aa, synthetic construct
MLQELPTLTPGQYSLVFNMFSFTVATMTASFVFFVLARNNVAPKYRISMMVSALVVFIAGYHYFRITSSWEAAYALQNGMYQPTGELFNDAYRYVNWLLTVPLLTVELVLVMGLPKNERGPLAAKLGFLAALMIVLGYPGEVSENAALFGTRGLWGFLSTIPFVWILYILFTQLGDTIQRQSSRVSTLLGNARLLLLATWGFYPIAYMIPMAFPEAFPSNTPGTIVALQVGYTIADVLAKAGYGVLIYNIAKAKSEEEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTKSRITSEGEYIPLDQIDINVFCYENEV

SEQ ID NO: 108 - protein, 581 aa, synthetic construct
MAKPTVKEIKSLQNFNRIAGVFHLLQMLAVLALANDFALPMTGTYLNGPPGTTFSAPVVILETPVGLAVALFLGLSALFHFIVSSGNFFKRYSASLMKNQNIFRWVQYSLSSSVMIVLIAQICGIADIVALLAIFGVNASMILFGWLQEKYTQPKDGDLLPFWFGCIAGIVPWIGLLIYVIAPGSTSDVAVPGFVYGIIISLFLFFNSFALVQYLQYKGKGKWSNYLRGERAYIVLSLVAKSALAWQIFSGTLIPAIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTKSRITSEGEYIPLDQIDINVFCYENEV

SEQ ID NO: 109 - protein, 593 aa, synthetic construct
MSSHSATTSSGRRESARDSRLRLWNSVMAVLHFLQGAAMVLLADTVLWPITRTRYGFDPGSQSIFPETVAFVDANLPLLVAGFLFISALAHTAIATVWYDKYVRYLDRGMNPYRWYEYSVSASLMIVVIGMLSGVWDLGTLVALFGLVAVMNLSGLLMEQRNELTEQTDWTPYWVGVIAGIVPWITIGVAFVGSVTASAGEFPEFVIYIYVSIFVFFNLFALNMALQYLEVSRWKNYLFGEKMYIVLSLVAKSALAWQVYFGTLNSPIIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTKSRITSEGEYIPLDQIDINVFCYENEV

SEQ ID NO: 110 - protein, 578 aa, synthetic construct
MTDYEDHTIRRFRIFNAIMGGIHLLQVFLVLYLSNNFSLPVTISKPVYNEFTNSISPVSETLFSIRVGPLVALFLFISAIAHILIATVLYYRYVENLKSGMNPYRWFEYSISASVMIVIIAMLTTIYDLGTLLALFTLTAVMNLMGLMMELHNQTTQNTDWTSYIIGCIAGLVPWIVIFIPLIAAESVPDFVIYIFVSIAIFFNCFAINMYLQYKKIGKWKDYLHGERVYIILSLVAKSALAWQVFAGTLRPMIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTKSRITSEGEYIPLDQIDINVFCYENEV

SEQ ID NO: 111 - protein, 572 aa, synthetic construct
MDTIVLTINWFNFIAGLFHVALAIVCALLGDVSQTFKMYMPFTTFRNGTQENEVTIKYSGYFPMTVIFVVYFSVTALFHMGNAFLWNDTYHRFLSNRKNPIRWTEYSITAPLMTAILAFIAGSRNWLFVIAASTLTFASISVGFVLDEIRKYSFFVLGMIPFLVEFSLIILSLYLTTSCYPSYLPITIFVEFGLWILFPFVSLLELSGINYKIGELVFIVLSFVSKSVLAIILNSENVLKNGVTGICIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTKSRITSEGEYIPLDQIDINVFCYENEV

SEQ ID NO: 112 - protein, 594 aa, synthetic construct
MTKALEMTILDDGSESPISFKYLRKFNLAMGILHLVQGVAMLVLGFFWDFSRPLYSITLDYSGGPPVAALQPEFSFTAVGPTVAAFLLFSALAHLLIAGPLNKFYVKNLKKKMNPIRWFEYAFSSSIMVFFIAILFGVWDLWVLIGLFFLNMLMNLFGHMMELHNQTTKKTNWTAYIYGWIAGIIPWVIISVFFARIAINSTGMPWFVPVIYAFELILFMSFAFNMLLQYKKVGKWKDYLYGERMYQILSLVAKTLLAWLVFAGVFQPAIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTKSRITSEGEYIPLDQIDINVFCYENEV

SEQ ID NO: 113 - protein, 563 aa, synthetic construct
MPEPGSEAIWLWLGTAGMFLGMLYFIARGWGETDSRRQKFYIATILITAIAFVNYLAMALGFGLTIVEFAGEEHPIYWARYSDWLFTTPLLLYNLGLLAGADRNTITSLVSLDVLMIGTGLVATLSPGSGVLSAGAERLVWWGISTAFLLVLLYFLFSSLSGRVADLPSDTRSTFKTLRNLVTVVWLVYPVWWLIGTEGIGLVGIGIVTAGFMVIDLTAKVGFGIILLRSHGVLDGAAIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTKSRITSEGEYIPLDQIDINVFCYENEV

SEQ ID NO: 114 - protein, 613 aa, synthetic construct
MIVDQFEEVLMKTSQLFPLPTATQSAQPTHVAPVPTVLPDTPIYETVGDSGSKTLWVVFVLMLIASAAFTALSWKIPVNRRLYHVITTIITLTAALSYFAMATGHGVALNKIVIRTQHDHVPDTYETVYRQVYYARYIDWAITTPLLLLNLGLLAGMSGAHIFMAIVADLIMVLTGLFAAFGSEGTPQKWGWYTIACIAYIFVVWHLVLNGGANARVKGEKLRSFFVAIGAYTLILWTAYPIVWGLADGARKIGVDGVIIAYAVLDVLAKGVFGAWLLVTHANLRESDIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTKSRITSEGEYIPLDQIDINVFCYENEV

SEQ ID NO: 115 - protein, 609 aa, synthetic construct
MIHHDQVAEMLYGYAGAAAAKPTHTDSPGPIPTVIPTPPQFQEIGETGHRTLWVVFALMVLSSGFFAFMSWNVPISKRLYHVITTLITITASLSYFAMASGHVTSFSCTPAKDHHKHVPDVGYTECRQVFWGRYVDWAITTPLLLLNLSLLAGIDGAHTLMAVIADVIMVLSGLFASQGETATQRWGWYAIGCVSYLFVIWHVALHGARTVTAKGRGVTRLFSSLALFTFVLWTAYPIVWGIADGAHRTTVDTVILIYAVLDILAKPVFGLWLLFSHRSLAETNIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTKSRITSEGEYIPLDQIDINVFCYENEV

SEQ ID NO: 116 - protein, 606 aa, synthetic construct
MIHPEQVADMLRPTTSTTSSHVPGPVPTVVPTPTEYQTLGETGHRTLWVTFALMVLSSGIFALLSWNVPTSKRLFHVITTLITVVASLSYFAMATGHATTFNCDTAWDHHKHVPDTSHQVCRQVFWGRYVDWALTTPLLLLQLCLLAGVDGAHTLMAIVADVIMVLCGLFAALGEGGNTAQKWGWYTIGCFSYLFVIWHVALHGSRTVTAKGRGVSRLFTGLAVFALLLWTAYPIIWGIAGGARRTNVDTVILIYTVLDLLAKPVFGFWLLLSHRAMPETNIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTKSRITSEGEYIPLDQIDINVFCYENEV

SEQ ID NO: 117 - protein, 560 aa, synthetic construct
MSNPNPFQTTLGTDAQWVVFAVMALAAIVFSIAVQFRPLPLRLTYYVNIAICTIAATAYYAMAVNGGDNKPTAGTGADERQVIYARYIDWVFTTPLLLLNLVLLTNMPATMIAWIMGADIAMIAFGIIGAFTVGSYKWFYFVVGCIMLAVLAWGMINPIFKEELQKHKEYTGAYTTLLIYLIVLWVIYPIVWGLGAGGHIIGVDVVIIAMGVLDLLAKPLYAIGVLITVEVVYGKIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTKSRITSEGEYIPLDQIDINVFCYENEV

SEQ ID NO: 118 - protein, 558 aa, synthetic construct
MAVHQIGEGGLVMYWVTFGLMAFSALAFAVMTFTRPLNKRSHGYITLAIVTIAAIAYYAMAASGGKALVSNPDGNLRDIYYARYIDWFFTTPLLLLNIILLTGIPIGVTLWIVLADVAMIMLGLFGALSTNSYRWGYYGVSCAFFFVVLWGLFFPGAKGARARGGQVPGLYFGLAGYLALLWFGYPIVWGLAEGSDYISVTAVAASYAGLDIAAKVVFGWAVMLSHPLIARNQIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTKSRITSEGEYIPLDQIDINVFCYENEV

SEQ ID NO: 119 - protein, 561 aa, synthetic construct
MVYSHADAAGWTLYWITYGIMAVTALIFFAMSLRRPIQQRSHHYTSFLIVAIASLAYYAMASQGGNTRIRVYQGPADSYRQIFWARYVDWFFTTPLLLLNLVLLSNLSKLRIAAIMVADIFMILTGLFGAVEARSNKWGWFVFGCIFMLYIFYELLVNVRKGAYARGGQHGMLYSVLLVWLLILWVQYPVVWGLAEGSSTVSSDTVIAWYAALDICAKCVFGFILLLGIESIDRKRIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTKSRITSEGEYIPLDQIDINVFCYENEV

SEQ ID NO: 120 - protein, 582 aa, synthetic construct
MSSLFEKRNTAVATNYRGPTNSIVINEAGSDWYWAVFSVMAASAIVFSVMAAMTPRGERVFHYLTIAIVSVASVAYFTMAADLGSVAIISEFANYSSLPTRQVFYARYIDWVITTPLLLTNLMLLAGLPWSTIIFTIVMDEVMVLTGLFGAITPSSYKWGYFTFGMVAYFFVAWVLIVEARKNAHRLGSDVHRLYIGIAIWTATLWTLYPVAWGLSEGGNVTSSDGVAIFYGVLDLLAKPVFGLWILLGHKGIGMDRIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTKSRITSEGEYIPLDQIDINVFCYENEV

SEQ ID NO: 121 - protein, 588 aa, synthetic construct
MESNLVRRNGALTVNTMTQNNQSVAIHQTVRGSDWYYTVCAVMGTTSLAILALSRMKPRTDRVFFYLTSGLCMVACIAYFAMGSNLGWTPIDVEWLRNDSVVRGVNRQVFYARYIDWVITTPMLLMNLLLTAGMPWPTILWIILLDEIMIVTGLIGALVKSRYKWGFYVFGCMAMFYIMWELAFPARKHAKVLGKDIHRSFVLCGVLTLVVWLCYPICWGLSEGGNVISPDSVSVFYGVLDVLAKPSFSIALIATHWNIDPGRIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTKSRITSEGEYIPLDQIDINVFCYENEV

SEQ ID NO: 122 - protein, 582 aa, synthetic construct
MGNSAIEVNGDTVDSYTADVKITTHGSDAYWAITAVMAFTTILFIAHSFTKPRTDRIFHYITISITLVASIAYFTMASNLGWASIFIEFQRDDPLVSGTTREIFYVRYIDWVITTPLLLLNILLTAGLPWPTILFTILLDEVMIITGLVGALVKSSYKWGFFTFGCVAFFGVAWSVAWTGRKHANALGSDIGRVYLMTSVWTLFLWLLYPIAWGVSEGGNVISPDSVAAFYGTLDVLAKPIFGIILLWGHRNIPASRIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTKSRITSEGEYIPLDQIDINVFCYENEV

SEQ ID NO: 123 - protein, 577 aa, synthetic construct
MDSLLLQRRNDAVATNPPNATFALTENGSSWLWAVFCVMALSCIIIAALSLFKPMGYRIFYLLNVAILATASVSYFSLASDLGLTPVTVEFRGPGTRQIAYVRYIDWFVTTPLLLTQLLLTAGLPTNIIISTIFADLVMIITGLAGALVVSRYKWGYYTMGCVAMLWVFWNVFTGIKVSGNIGPDVRKSYTLLAVWLMIIWLNYPICWGLAEGGNRITVVGVMVYYGVLDLLAKPVFAAISLAVHSKIELSRIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTKSRITSEGEYIPLDQIDINVFCYENEV

SEQ ID NO: 124 - protein, 592 aa, synthetic construct
MDAILSKRNEVLSLNPLVANIDITTAASDWLWAVFAVMGLSAIILLVLGHATRPIGERAFHELAAALCFTASIAYYSMASDLGATPIEVEFIRGGTLGQNWVDIGVLRPTRSIWYARYIDWTITTPLLLLQLALTTALPLSQIFGLVFFDIVMIITGLLGALTASRYKWGFFVFGCVAMFWIFWVLFFPARKSASHLGTDYHRAYTSSAIVLCTLWTVYPIIWGVCDGGNVITPTSVMVAYGVLDLLAKPVFSFWHVFQLSRLDYARIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTKSRITSEGEYIPLDQIDINVFCYENEV

SEQ ID NO: 125 - protein, 581 aa, synthetic construct
MPFYIKNADIDITTHGSDWLWAVFSVMLLSAIGILVWGHVARPLGERAFHELAAALCFTASIAYFAMASDLGDVPIVVEFIRGGSLGQNWVQVGVENPTRAIWYARYIDWTITTPMLLLQLLLCTGLPLSQVFSVIFADLLMIETGLIGALVASRYKWGFYAFGCAAQLYIWWMLLVPGRRSAQHIGSDFAKSYTMSNIFLTTVWLVYPVIWGVADGGNVITPDSVMIAYGVLDLLAKPVFSVIHLMSLSKLDYARIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTKSRITSEGEYIPLDQIDINVFCYENEV

SEQ ID NO: 126 - protein, 567 aa, synthetic construct
MKFLLLLLADPTKLDPSDYVGFTFFVGAMAMMAASAFFFLSLNQFNKKWRTSVLVSGLITFIAAVHYWYMRDYWFAIQESPTFFRYVDWVLTVPLMCVQFYLILKVAGAKPALMWKLILFSVIMLVTGYFGEAVFQDQAALWGAISGAAYFYIVYEIWLGSAKKLAVAAGGDILKAHKILCWFVLVGWAIYPLGYMLGTDGWYTSILGKGSVDVAYNIADAINKIGFGLVIYALAVKKNEVDIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTKSRITSEGEYIPLDQIDINVFCYENEV

SEQ ID NO: 127 - protein, 598 aa, synthetic construct
MTQELGNANFENFIGATEGFSEIAYQFTSHILTLGYAVMLAGLLYFILTIKNVDKKFQMSNILSAVVMVSAFLLLYAQAQNWTSSFTFNEEVGRYFLDPSGDLFNNGYRYLNWLIDVPMLLFQILFVVSLTTSKFSSVRNQFWFSGAMMIITGYIGQFYEVSNLTAFLVWGAISSAFFFHILWVMKKVINEGKEGISPAGQKILSNIWILFLISWTLYPGAYLMPYLTGVDGFLYSEDGVMARQLVYTIANVSSKVIYGVLLGNLAITLSKNKIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTKSRITSEGEYIPLDQIDINVFCYENEV

SEQ ID NO: 128 - protein, 600 aa, synthetic construct
MLMTVFSSAPELALLGSTFAQVDPSNLSVSDSLTYGQFNLVYNAFSFAIAAMFASALFFFSAQALVGQRYRLALLVSAIVVSIAGYHYFRIFNSWDAAYVLENGVYSLTSEKFNDAYRYVDWLLTVPLLLVQTVAVLTLPAKEARPLLIKLTVASVLMIATGYPGEISDDITTRIIWGTVSTIPFAYILYVLWVELSRSLVRQPAAVQTLVRNMRWLLLLSWGVYPIAYLLPMLGVSGTSAAVGVQVGYTIADVLAKPVFGLLVFAIALVKTKADIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTKSRITSEGEYIPLDQIDINVFCYENEV

SEQ ID NO: 129 - protein, 583 aa, synthetic construct
MLQELPTLTPGQYSLVFNMFSFTVATMTASFVFFVLARNNVAPKYRISMMVSALVVFIAGYHYFRITSSWEAAYALQNGMYQPTGELFNDAYRYVDWLLTVPLLTVQLVLVMGLPKNERGPLAAKLGFLAALMIVLGYPGEVSENAALFGTRGLWGFLSTIPFVWILYILFTQLGDTIQRQSSRVSTLLGNARLLLLATWGFYPIAYMIPMAFPEAFPSNTPGTIVALQVGYTIADVLAKAGYGVLIYNIAKAKSEEEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTKSRITSEGEYIPLDQIDINVFCYENEV

SEQ ID NO: 130 - protein, 592 aa, synthetic construct
MTMLEHLEGTMDGWYAENDLGQGAIIAHWVTFFFHMITTFYLGYVSFHSKGPGGKQPYFAGYHEENNIGIFVNLFAAISYFGKVVSDTHGHNYQNVGPFIIGLGNYRYADYMLTCPLLVMNLLFQLRAPYKITCAMLIFAVLMIGAVTNFYPGDDMKGPAVAWFCFGCFWYLIAYIFMAHIVSKQYGRLDYLAHGTKAEGALFSLKLAIITFFAIWVAFPLVWLLSVGTGVLSNEAAEICHCICDVVAKSVYGFALANFREQYDRELIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTKSRITSEGEYIPLDQIDINVFCYENEV

SEQ ID NO: 131 - protein, 566 aa, synthetic construct
MVESSAVIAANWISFLVIAGSFVVLCFISLRYKGPGGNENYYNGFREQNMLTVIINLWCALAYFAKVLQSHSDDDGFVPLTKIPYLDYATTCPLLTLNLMWCLDAPYKITSAVLVFTVMITGVACSLAVAPYSFYWFAMGMVLFIFTYVLMLSIVRERLEFITQCAHDSNAKRSIKHLKAAVIIYFGIWPIFAILWLLSYRAANVISNDTNHILHCILDVIAKSCFGFVLLHFKMYFDKKLIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTKSRITSEGEYIPLDQIDINVFCYENEV

SEQ ID NO: 132 - protein, 566 aa, synthetic construct
MVASSAVITANWISFLAISASFIILLVISLRYKGPGGTESFYNGFKEQNMLTVFINLWCALAYFAKVLQSHSNDNGFAPLTVIPYVDYCTTCPLLTLNLLWCLDAPYKISSAVLVFTCLVIAVACSLAVAPFSYCWFAMGMVLFTFTYVFILSIVRQRLDFFTLCARDSNAKQSLKHLKTAVFIYFGIWLLFPLLWLLSYRAANVISNDINHIFHCILDVIAKSVYGFALLYFKMYFDKKLIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTKSRITSEGEYIPLDQIDINVFCYENEV

SEQ ID NO: 133 - protein, 572 aa, synthetic construct
MVSALDQNGPQYLQNPIVIAADWIGFIALFGSSLAVAYKLVTFKGPDQDDVYFFGYREEKMISVFVNLFAALAYWAKLASHANGDVGPAASVTTYKYLDYLFTCPLLTINLLWCLNLPYKFTFGAIVAVCILCAFMASVIPPPARYMWFGMGITVFSAAWFNILKLVRMRLEQFVSKEAKKVRQSLKVACMTYFFIWLGYPTLWVLGDAGVLDSVVSALLHTFLDVFSKSIYGFALLHFVMRTDKREIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTKSRITSEGEYIPLDQIDINVFCYENEV

SEQ ID NO: 134 - protein, 585 aa, synthetic construct
MTTSAPSLSDPNWQYGMGGWNNPRLPNFNLHDPTVIGVDWLGFLCLLGASLALMYKLMSFKGPDGDQEFFVGYREEKCLSIYVNLIAAITYWGRICAHFNNDMGLSLSVNYFKYLDYIFTCPILTLNLLWSLNLPYKITYSLFVGLTIACNAFEPPARYLWFMFGCFIFAFTWISIIRLVYARFQQFLNEDAKKIRAPLKLSLTLYFSIWCGYPALWLLTEFGAISQLAAHVMTVIMDVAAKSVYGFALLKFQLGVDKRDIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTKSRITSEGEYIPLDQIDINVFCYENEV

SEQ ID NO: 135 - protein, 586 aa, synthetic construct
MSTSSVAYLRTPVVQALDWVGFISLGGTAAVLAYRLMNFKPPNKDILYFFGYREKGMISLYVNLFAAVAYYARITSHLSGDVGAATNIILYKYFDYLITCPLLTFNLLTTLNLPYKITYAVYVQITIFTGFMSANTPPPATFLWFAFGMLLFSYTWFNIISLVQVRFIQYFAKKGNTTQSRRVSVASKAGFRNKNVRNPLQTALSTYFCIWMVYPVLWLLLKTKVIDQVTEHCINVVMDVLAKSMYGFALLRFQLLMDKANIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEPTTKSRITSEGEYIPLDQIDINVFCYENEV